(19) United States
(12) Patent Application Publication
(10) Pub. No.: US 2003/0143200 A1
(43) Pub. Date: Jul. 31, 2003

(54) Porcine adenovirus E1 region

(76) Inventor: Suresh K. Tikoo, Saskatoon (CA)

Correspondence Address: MORRISON & FOERSTER LLP, 755 PAGE MILL RD, PALO ALTO, CA, 94304-1018, US

(21) Appl. No.: 09/963,038

(22) Filed: Sep. 24, 2001

(51) Int. Cl. A61K 48/00; C12N 7/00; C12N 15/869

(52) U.S. Cl. 424/93.2; 514/44; 435/456; 435/235.1; 435/320.1

(57) ABSTRACT

The present invention relates to the characterization of the porcine adenovirus E1 region. The complete nucleotide sequence of the genome of porcine adenovirus type 3 (PAV-3), providing the characterization of the PAV3 E1 region, is described herein. Methods for construction of infectious PAV genomes by homologous recombination in procaryotic cells are provided. Recombinant PAV viruses are obtained by transfection of mammalian cells with recombinant PAV genomes. The PAV-3 genome can be used as a vector for the expression of heterologous nucleotide sequences, for example, for the preparation and administration of subunit vaccines to swine or other mammals.

### TECHNICAL FIELD

The present invention is in the field of recombinant mammalian viral vectors. More particularly, it concerns recombinant porcine adenovirus vectors for diagnostic and therapeutic purposes, such as vaccines and expression systems.

### BACKGROUND

Adenoviruses are double-stranded DNA viruses that have been isolated from a wide variety of avian and mammalian species, including swine. Porcine adenoviruses (PAV) belong to the Mastadenovirus genus of Adenoviridae family. Of the five serotypes identified till date (Derbyshire et al., 1975, J. Comp. Pathol. 85:437-443; Hirahara et al., 1990, Japanese J. Vet Sci. 52:407-409), serotype 3 (PAV-3) could propagate to high titers in cell culture. While the majority of adenovirus infections in swine are subclinical, porcine adenovirus (PAV) infection has been associated with encephalitis, pneumonia, kidney lesions and diarrhea Derbyshire (1992) In: “Diseases of Swine” (ed. Leman et al.), 7th edition, Iowa State University Press, Ames, IA. pp. 225-227. Thus, there is a need for vaccines that will provide protection against PAV infection.

In addition to their potential ability to provide protection against PAV infection, PAVs could also be used as viral vaccine vectors, if insertion capacity can be determined, and appropriate insertion sites can be defined and characterized. It has been shown that PAV is capable of stimulating both humoral response and a mucosal antibody responses in the intestine of infected piglets. Tuboly et al. (1993) Res. in Vet. Sci. 54:345-350. Thus, recombinant PAV vaccine vectors would be especially useful, as they would be likely to be capable of providing both systemic and mucosal immunity to antigens encoded by native and/or recombinant PAV genomes.

Cross-neutralization studies have indicated the existence of at least five serotypes of PAV. Derbyshire et al. (1975) J. Comp. Pathol. 85:437443; and Hirahara et al. (1990) Jpn. J. Vet. Sci. 52:407-409. Previous studies of the PAV genome have included the determination of restriction maps for PAV Type 3 (PAV-3) and cloning of restriction fragments representing the complete genome of PAV-3. Reddy et al. (1993) Intervirology 36:161-168. In addition, restriction maps for PAV-1 and PAV-2 have been determined. Reddy et al. (1995b) Arch. Virol. 140:195-200.

Nucleotide sequences have been determined for segments of the genome of various PAV serotypes. The transcription map and complete DNA sequence of PAV-3 genome was reported (Reddy et al., 1998, Virus Res, 58:97-106 and Reddy et al., 1998, Virology 251:414-426). Sequences of the E3, pVIII and fiber genes of PAV-3 were determined by Reddy et al. (1995a) Virus Res. 36:97-106. The E3, pVIII and fiber genes of PAV-1 and PAV-2were sequenced by Reddy et al. (1996) Virus Res. 43:99-109; while the PAV4 E3, pVIII and fiber gene sequences were determined by Kleiboeker (1994) Virus Res. 31:17-25. The PAV4 fiber gene sequence was determined by Kleiboeker (1995b) Virus Res. 39:299-309. Inverted terminal repeat (ITR) sequences for all five PAV serotypes (PAV-1 through PAV-5) were determined by Reddy et al. (1995c) Virology 212:237-239. The PAV-3 penton sequence was determined by McCoy et al. (1996a) Arch. Virol. 141:1367-1375. The nucleotide sequence of the E1 region of PAV4 was determined by Kleiboeker (1995a) Virus Res. 36:259-268. The sequence of the protease (23K) gene of PAV-3 was determined by McCoy et al. (1996b) DNA Seq. 6:251-254. The sequence of the PAV-3 hexon gene (and the 14 N-terminal codons of the 23K protease gene) has been deposited in the GenBank database under accession No. U34592. The unpublished sequence of the PAV-3 100K gene has been deposited in the GenBank database under accession No. U82628. The sequence of the PAV-3 E4 region has been determined by Reddy et al. (1997) Virus Genes 15:87-90.

Adenoviruses have proven to be effective vectors for the delivery and expression of foreign genes in a number of specific applications, and have a number of advantages as potential gene transfer and vaccine vectors. See Gerard et al (1993) Trends Cardiovasc. Med. 3:171-177; Imler et al. (1995) Hum. Gene Ther. 6:711-721. The ability of these vectors to mediate the efficient expression of candidate therapeutic or vaccine genes in a variety of cell types, including post mitotic cells, is considered an advantage over other gene transfer vectors. Adenoviral vectors are divided into helper-independent and helper-dependent groups based on the region of the adenoviral genome used for the insertion of transgenes. Helper-dependent vectors are usually made by deletion of E1 sequences and substitution of foreign DNA, and are produced in complementing human cell lines that constitutively express E1 proteins. Graham et al. (1977) J. Gen. Virol. 36:59-74; Fallaux et al. (1996) Hum. Gene Ther. 7:215-222; Fallaux et al. (1998) Hum. Gene Ther. 9:1909-1917. However, porcine adenoviruses do not replicate in human cell lines; hence these lines are unsuitable for the propagation of E1-deleted PAV vectors. E1A region is described in Darbysbire (1966, Nature 211:102) and Whyte et al., 1988, J. Virol. 62:257-265.

Though E1-deleted viruses do not replicate in cells that do not express E1 proteins, the viruses can express foreign proteins in these cells, provided the genes are placed under the control of a constitutive promoter. xiang et al. (1996) Virology 219:220-227. Vaccination of animals with adenovirus recombinants containing inserts in the E1 region induced a systemic immune response and provided protection against subsequent challenge. Imler et al. (1995) Hum. Gene Ther. 6:711-721; Inter et al. (1996) Gene Therap 3:75-84.. This type of expression vector provides a significant safety profile to the vaccine as it eliminates the potential for dissemination of the vector within the vaccine and therefore, the spread of the vector to non-vaccinated contacts or to the general environment. However, the currently used human adenovirus (HAV) based vectors are endemic in most populations, which provides an opportunity for recombination between the helper-dependent viral vectors and wild type viruses. To circumvent some of the problems associated with the use of human adenoviruses, non human adenoviruses have been explored as possible expression vectors.

Use of vectors containing an intact E1 region for gene therapy in humans and vaccination in animals is unsafe because they have the ability to replicate in normal cells and spread to other animals, and they retain any oncogenic potential of the E1 region. WO 99/53047 disclose the use of PAV vectors deleted in their E1 region. See Klonjkowski et al (1997) Hum. Gene Ther. 8:2103-2115 which discloses E1 deleted canine adenovirus 2.

There remains a need for improved adenoviral vectors lacking E1 replication and oncogenic functions, for expression of transgenes in mammalian cells, and for the development of effective recombinant PAV vectors for use in immunization and expression systems.

### SUMMARY OF THE INVENTION

The present invention relates to the characterization of the porcine adenovirus region. The present invention discloses the complete nucleotide sequence of the genome of porcine adenovirus type 3 (PAV-3) and provides the characterization of the PAV3 E1 region, including E1A, E1B^{small} and E1B^{large}. Nucleic acid sequences that are substantially homologous to those comprising a PAV genome are also encompassed by the invention. Substantially homologous sequences include those capable of duplex and/or triplex formation with a nucleic acid comprising all or part of a PAV genome (or with its complement). As is known to those of skill in the art, duplex formation is influenced by hybridization conditions, particularly hybridization stringency. Factors affecting hybridization stringency are well-known to those of skill in the art. See, for example, Sambrook et al. (1989) Molecular Cloning: A Laboratory Manual; Hames et al. 1985) Nucleic Acid Hybridisation: A Practical Approach, IRL Press Ltd., Oxford Accordingly, it is within the skill of the art to identify a sequence that is substantially homologous to a sequence from a PAV genome.

In particular, the present invention provides a replication-defective recombinant PAV vector, comprising at least one heterologous nucleotide sequence, wherein the PAV vector lacks E1A and/or E1B^{large} function and retains E1B^{small} function. In some embodiments, the vector comprises a deletion of part or all of the E1A and/or E1B^{large} gene region. In other embodiments, the vector comprises an insertion in the E1A and/or E1B^{large} gene region that inactivates the E1A and/or E1B^{large} region function. In some embodiments, the vector further comprises a deletion of part or all of the E3 region, or other non-essential regions of the adenovirus. In additional embodiments, the PAV is PAV3.

In yet other embodiments, the present invention provides a replication-defective recombinant PAV vector that comprises a deletion in the E1 region that consists of a deletion of the E1A and/or E1B^{small} region. In yet other embodiments, the present invention provides a replication-defective recombinant PAV vector that comprises an insertion in the E1 region that consists of an insertion in the E1A and/or E1B^{large} region that inactivates E1A and/or E1B^{large} region function.

The present invention also provides a replication-defective recombinant PAV vector comprising at least one heterologous nucleotide sequence, wherein the PAV vector lacks E1A function and E1B^{small} function and retains E1B^{large} function. In some embodiments, the vector comprises a deletion of part or all of the E1A and E1B^{small} regions. In other embodiments, the vector comprises an insertion that inactivates the E1A gene region function. In further embodiments, the vector has a deletion of part or all of the E3 region, or other non-essential regions of the adenovirus.

In further embodiments, the present invention provides a PAV vector comprising at least one heterologous nucleotide sequence, wherein said vector lacks E1B^{small} function and retains E1A and E1B^{large} function. In some embodiments, the vector comprises a deletion of part or all of the E1BSₛₘₐₗₗ region. In further embodiments, the vector comprises a deletion in the E3 region or other non-essential regions. In additional embodiments, the PAV is PAV3.

In further embodiments, the heterologous nucleotide sequence encodes a therapeutic polypeptide. In yet further embodiments, the heterologous polypeptide sequence encodes an antigen. In yet further embodiments, the therapeutic polypeptide is selected from the group consisting of coagulation factors, growth hormones, cytokines, lymphokines, tumor-suppressing polypeptides, cell receptors, ligands for cell receptors, protease inhibitors, antibodies, toxins, immunotoxins, dystrophins, cystic fibrosis transmembrane conductance regulator (CFTR), immunogenic polypeptides and vaccine antigens.

The present invention also provides host cells infected with a recombinant PAV vector of the present invention. The present invention also provides methods for producing a recombinant PAVs that comprises introducing a PAV vector that lacks E1A function and/or E1B^{large} function and retains E1B^{small} function into a helper cell line that expresses E1A function and/or E1B^{large} function and recovering virus from the infected cells. In one embodiment, the present invention comprises introducing a PAV vector that lacks E1A function, and retains E1B^{small} and E1B^{large} function, into a helper cell line that expresses E1A function. In some embodiments, the helper cell line expresses human E1A function.

The present invention also provides recombinant mammalian cell lines that comprise nucleic acid encoding mammalian adenovirus E1A function and lack nucleic acid encoding mammalian adenovirus E1B^{small} function. In some embodiments, the E1A function is human E1A function. The present invention also provides recombinant mammalian cell lines that comprise nucleic acid encoding mammalian adenovirus E1B^{large} function and lack nucleic acid encoding mammalian adenovirus E1Bₛₘₐₗₗ function. In some embodiments, the E1B^{large} function is human E1B^{large} function. In some embodiments, the cell line is of porcine origin.

The present invention also provides methods for producing a recombinant PAV that lacks E1A and retains E1B^{small} function. In one embodiment, the present invention provides a method comprising introducing, into an appropriate helper cell line, a porcine adenovirus vector comprising ITR sequences, PAV packaging sequences, and at least one heterologous nucleotide sequence, wherein said vector lacks E1A function and retains E1B^{small} function; culturing the cell line under conditions whereby adenovirus virus replication and packaging occurs; and recovering the adenovirus from the infected cells. In some embodiments, the PAV is PAV3.

The present invention also provides methods for producing a recombinant PAV that lacks E1B^{small} function and retains E1A and/or E1B^{large} function.

In additional embodiments, the invention provides compositions that are able to elicit an immune response or able to provide immunity to PAV infection, through expression of antigenic PAV polypeptides. The invention also provides vectors comprising PAV genome sequences, including sequences encoding various PAV genes as well as PAV regulatory sequences, which are useful for controlling the expression of heterologous genes inserted into PAV vectors.

### BRIEF DESCRIPTION OF THE DRAWINGS

FIGS. 1-1 through 1-10 show the complete nucleotide sequence of the PAV-3 genome (SEQ ID NO: 1).

FIG. 2 shows the transcriptional map of the PAV-3 genome derived from alignment of the sequences of cDNA clones with the genomic sequence, and nuclease protection mapping of viral transcripts. The PAV-3 genome is represented by the thick horizontal line, with the numbers below the line representing PAV-3 map units (i.e., percentage of genome length from the left end). Rightward-reading transcription units are depicted above the line and leftward-reading transcription units are shown below the line.

FIGS. 3A-3B show immunoprecipitation of E1A and E1B proteins from various cell lines. In FIG. 3A, proteins in cell lysates were separated by gel electrophoresis, and analyzed by immunoblotting using the DP11 monoclonal antibody, which recognizes the human adenovirus E1A protein. Lane 1: 293 cells (human cells transformed by HAV-5, which express adenovirus E1A and E1B); Lane 2: Fetal porcine retinal cells; Lane 3: VIDO R1 cells; Lane 4: 293 cells. In FIG. 3B, proteins in cell lysates were separated by gel electrophoresis, and analyzed by immunoblotting using the DP17 monoclonal antibody, which recognizes the human adenovirus E1B protein. Lane 1: human 293 cells; Lane 2: Fetal porcine retinal cells; Lane 3: VIDO R1 cells; Lane 4: 293 cells.

FIG. 4 shows a map of the plasmid pPAV-101.

FIG. 5 shows a map of the plasmid pPAV-102.

FIG. 6 shows a map of the plasmid pPAV-300.

FIG. 7 shows proteins labeled after infection of VIDO R1 cells with a recombinant PAV containing the PRV gp50 gene inserted in the E3 region. Labeled proteins were separated by gel electrophoresis; an autoradiogram of the gel is shown. Lane 1: Molecular weight markers of 30K, 46K, 69K and 96K, in order of increasing molecular weight. Lane 2: Mock-infected cells, 12 hours post-infection. Lane 3: PAV-3-infected cells, 12 hours post-infection. Lane 4: cells infected with a recombinant PAV containing the PRV gp50 gene, 12 hours post-infection. Lane 5: cells infected with a recombinant PAV containing the PRV gp50 gene, 16 hours post-infection. Lane 6: cells infected with a recombinant PAV containing the PRV gp50 gene, 24 hours post-infection.

FIG. 8 provides a schematic diagram of the construction of an E1- and E3-deleted PAV vector with a green fluorescent protein gene insertion.

FIGS. 9A-9F provide a schematic representation of strategies used for generation of porcine genomic DNA in plasmids. (Figure A) plasmid pPAVXhoIRL; (Figure B) plasmid pFPAV211; (Figure C) plasmid pFPAV212; (Figure D) plasmid pFPAV507; (Figure E) plasmid pFPAV214; (Figure F) plasmid pFPAV216. ITR (filled box); The origin of DNA sequences is as follows: BAV-3 genome (open box); AmpR gene (arrow); plasmid DNA (broken line). The plasmid maps are not drawn to scale.

FIG. 10 shows the immunoprecipitation of proteins synthesized by in vitro transcription and translation of plasmids. [³⁵S]-methionine labeled in vitro transcribed and translated pSP64PE1A (lanes 7,9), pSP64-PE1Bs (lanes 4,6), pSP64-PE1B1 (lanes 1,3) and pSP64polyA (lanes 2,5,8) products before (lanes 3,6,9) and after immunoprecipitation with anti-E1A (lanes 8,9), anti-E1B^{small} (lanes 5,6) and anti-E1B^{large} (lanes 2,3) were separated on 10% SDS-PAGE gels under reducing conditions. The positions of the molecular weight markers are shown to the left of the panel.

FIG. 11 shows the in vivo immunoprecipitation of E1 proteins. Proteins from the lysates of [³⁵S] methionine-cysteine labeled mock (lane 3) or PAV3 infected (lane 1, 6 h post infection; lane 2, 24 h post infection) VIDO R1 cells were immunoprecipitated with anti-E1A serum (panel A), anti-E1B^{small} serum (panel B), anti-E1B serum (panel C) and separated on 10% SDS-PAGE under reducing conditions. The positions of the molecular weight markers are indicated to the left of each panel.

FIGS. 12A-12C provide the restriction enzyme analysis of recombinant PAV-3 genome. (Figure A)The viral DNAs were extracted from VIDO R1 cells infected with PAV211 (lane 1), PAV212 (lane 2) or wild-type PAV-3 (lane 3) and digested with SpeI. Sizes of marker (M) are shown in basepairs. (Figure B) The viral DNAs were extracted from VIDO R1 cells infected with PAV214 (lane 1) or wild-type PAV-3 (lane 2) and digested with NheI. Sizes of marker (M) are shown in base pairs. (Figure C) The viral DNAs were extracted from VIDO R1 cells infected with PAV216 (lane 2) or wild-type PAV-3 (lane 1) and digested with AseI. Sizes of marker (M) are shown in base pairs.

FIG. 13. Western blot analysis of PAV-3 protein expression in mutant infected cells. Proteins from wild-type PAV3 (lane 3), PAV211 (lane 2), or PAV212 (lane 1) infected ST cells were separated by 12.5% SDS-PAGE under reducing conditions and transferred to nitrocellulose. The separated proteins were probed in Western blots by anti-E1A (panel C), anti-E1B^{small} (panel A) or anti-DBP (panel B). The positions of the molecular weight markers are shown to the left of each panel.

FIG. 14. Western Blot analysis of GFP expression. Proteins from purified GFP (lane 2) or mock (lane 1), wild-type PAV-3 (lane 3) and PAV216 (lane 4 and 5) infected VIDO R1 cells harvested at 24 h.p.i (lane 3, 4) and 48 h.p.i. (lane 5) were separated by 10% SDS-PAGE under reducing conditions and transferred to nitrocellulose. The separated proteins were probed Western blots by anti-GFP polyclonal antibody.

FIGS. 15A-15B. Virus titers of recombinant and wild-type PAV-3. Near-confluent monolayers of VIDO R1 (Figure A) or Swine Testicular (ST) (Figure B) cells were infected with recombinant or wild-type PAV-3. At different time points post infection, the cell pellets were freeze-thawed and virus was titrated on VIDO R1 cells as described in the text.

### DETAILED DESCRIPTION OF THE INVENTION

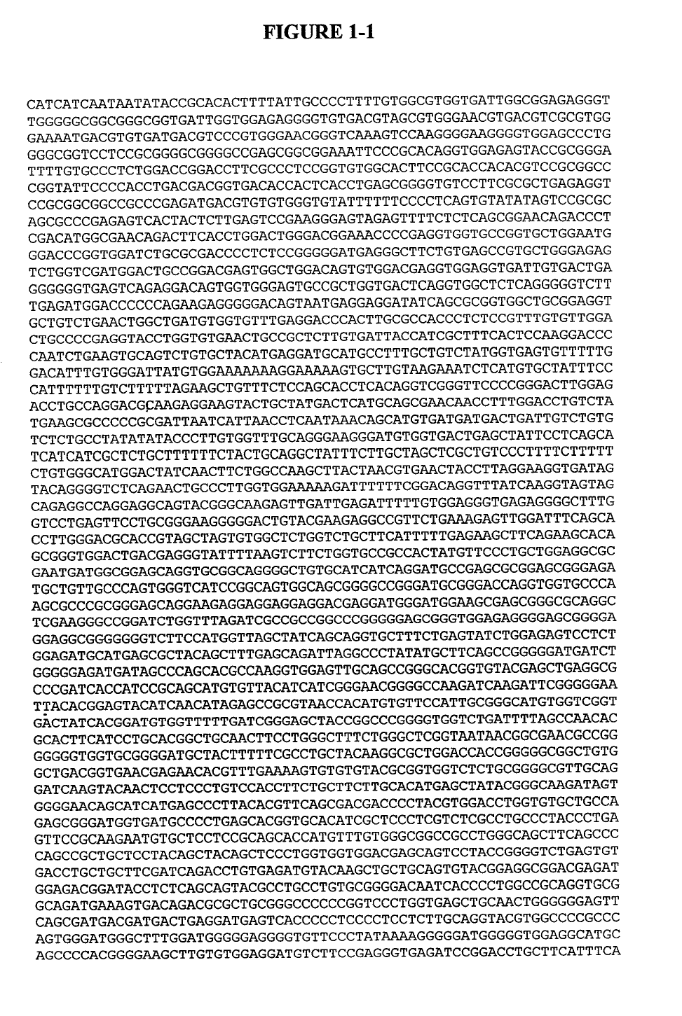

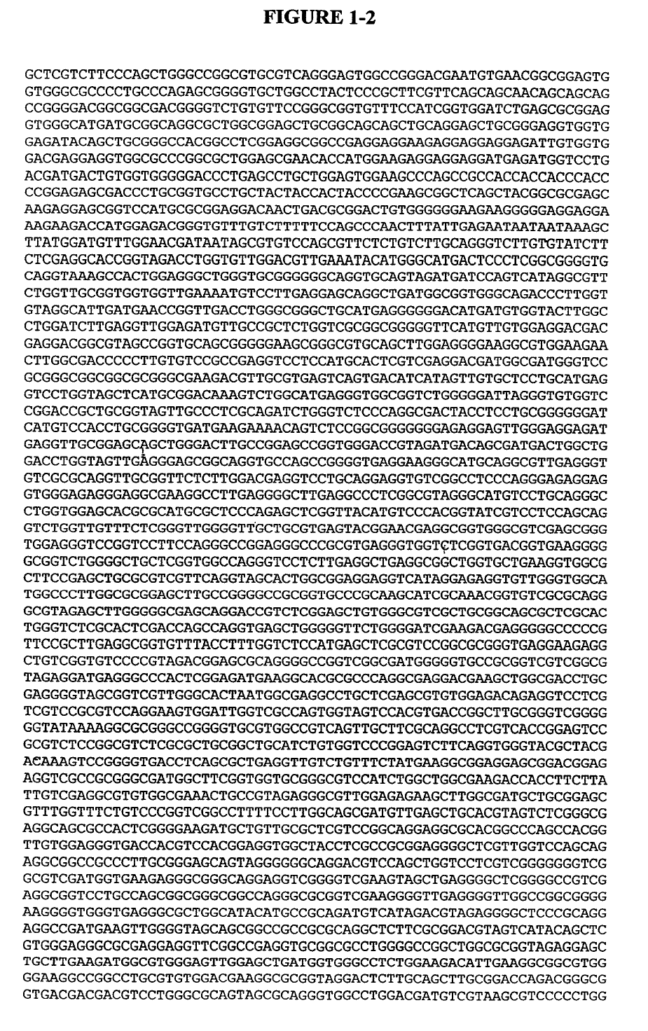

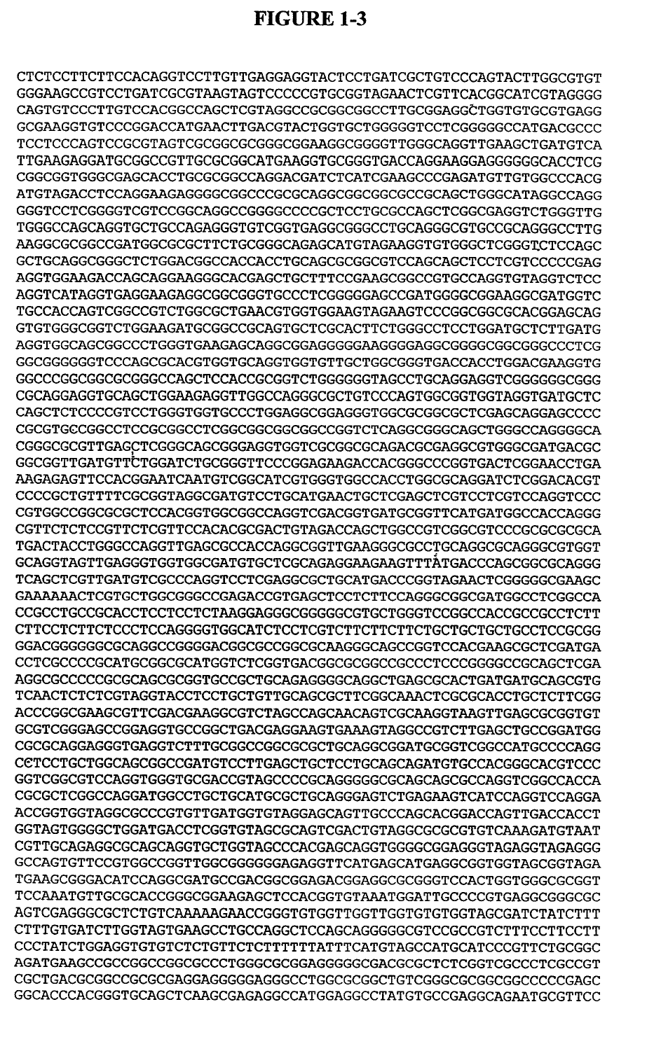

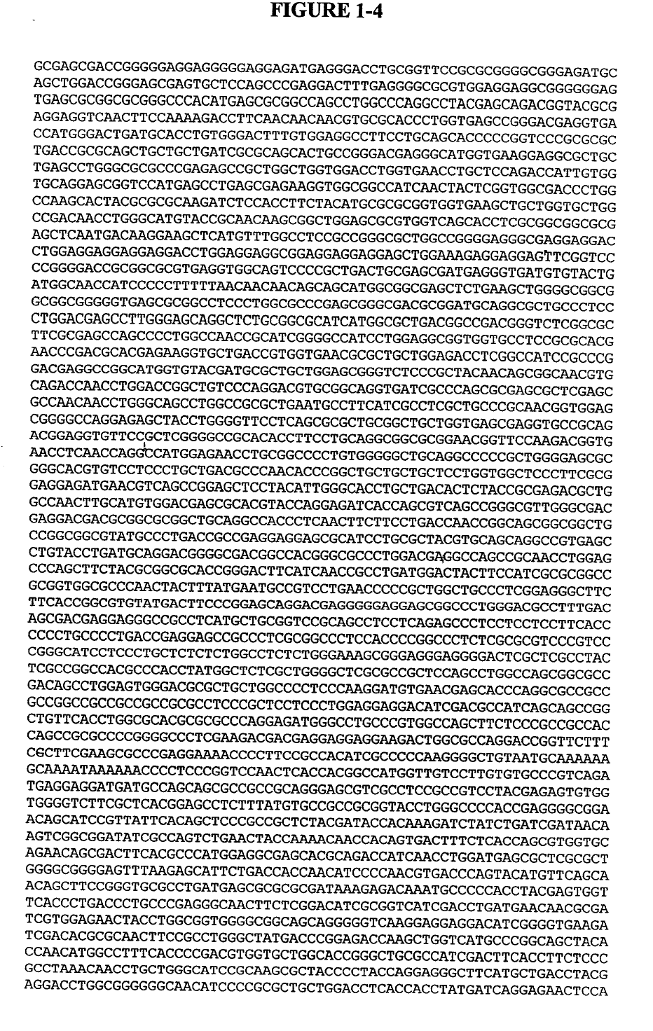

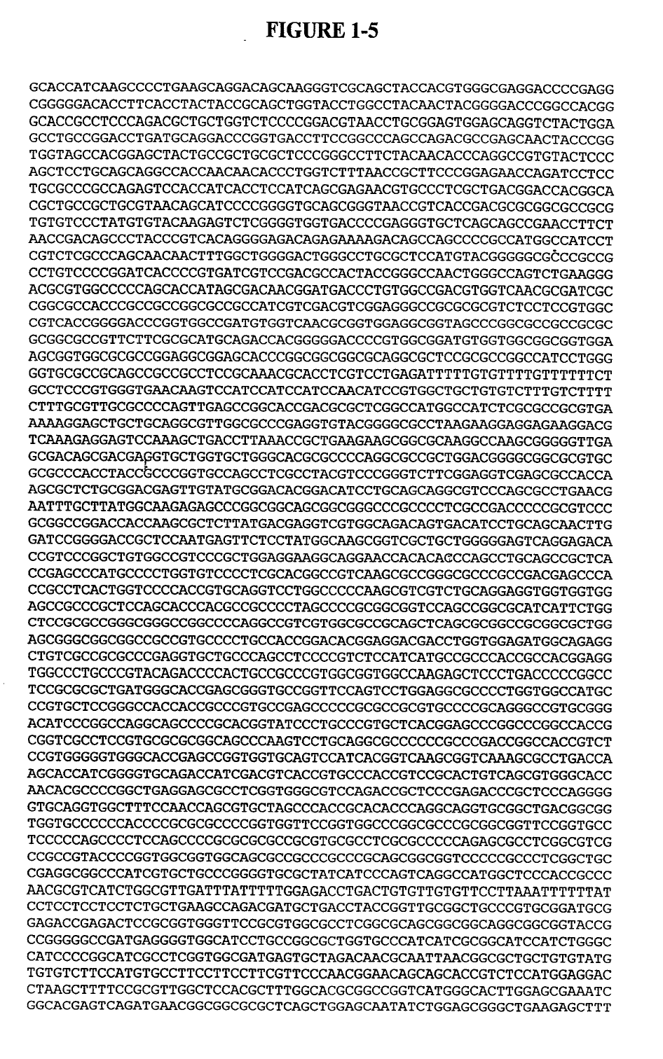

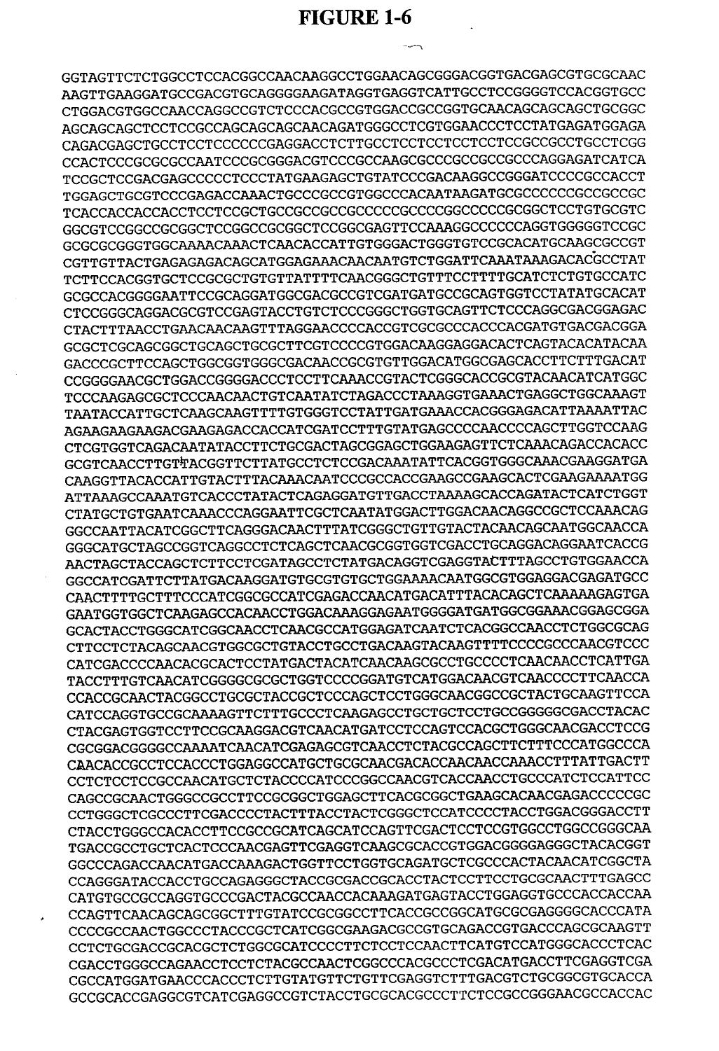

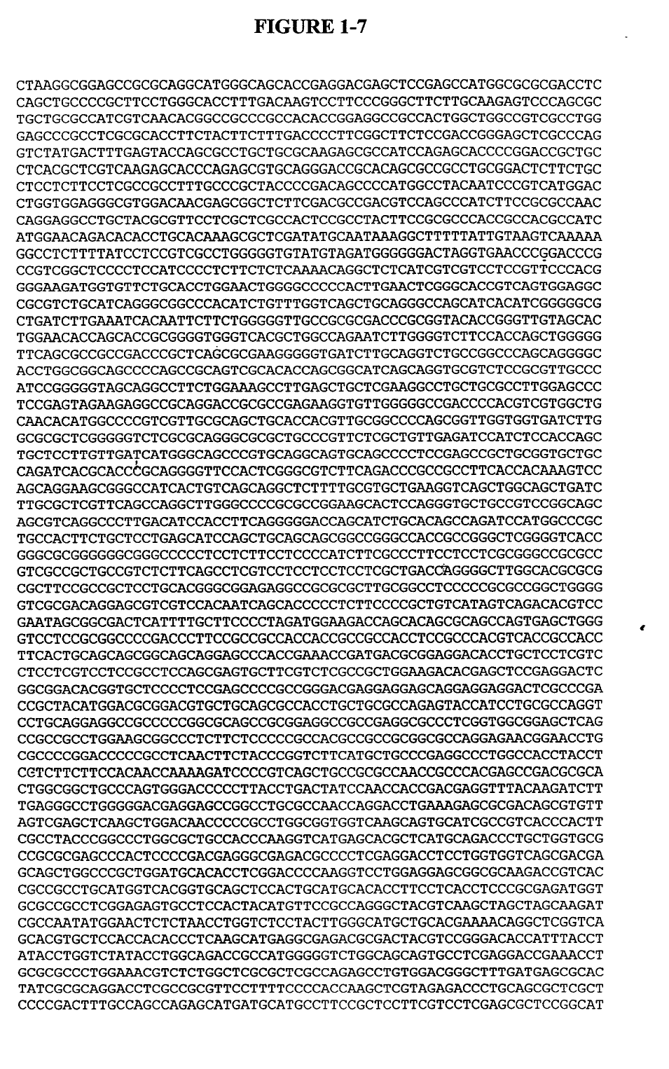

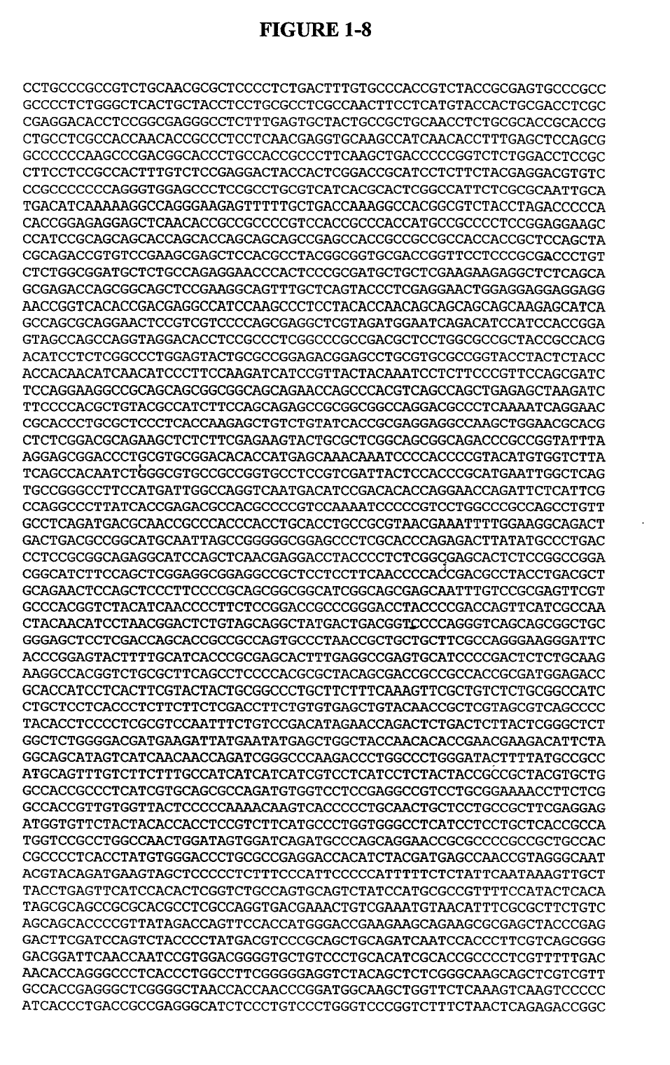

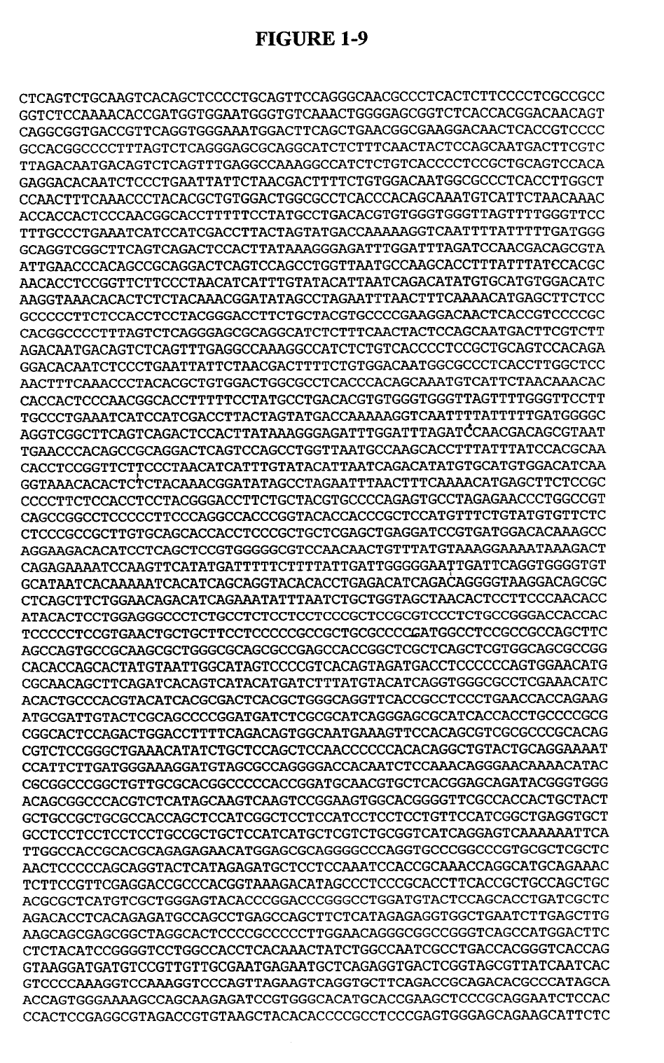

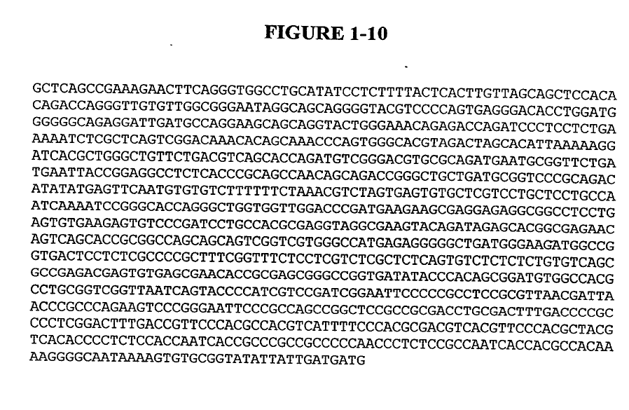

The present invention provides the complete nucleotide sequence and transciptional map of the porcine adenovirus type 3 (PAV-3) genome and the characterization of the E1 region of PAV3. In particular, the inventors have discovered that E1A and E1B^{large} regions are essential for virus replication and E1B^{small} is non-essential for virus replication. The PAV3 nucleotide sequence comprises a linear, double-stranded DNA molecule of about 34,094 base pairs, as shown in FIG. 1 (SEQ ID NO: 1). Previously-determined partial sequences can be aligned with the complete genomic sequence as shown in Table 1.

TABLE 1
Alignment of published PAV-3 sequences

| | PAV Genes(s) | | |
|---|---|---|---|
| GenBank | included | | Genome |
| Accession No. | within sequence | Reference | coordinates |
| L43077 | ITR | Reddy et al., 1995c | 1-144 |
| U24432 | penton | McCoy et al., 1996a | 13556-15283 |
| U34592 | hexon; N-terminal | unpublished | 19036-21896 |
| | 14 codons of 23 K | | |
| | (protease) gene | | |
| U33016 | protease (23 K) | McCoy et al., 1996b | 21897-22676 |
| U82628 | 100 K | unpublished | 24056-26572 |
| U10433 | E3, pVllI, fiber | Reddy et al., 1995a | 27089-31148 |
| L43363 | E4 | Reddy et al., 1997 | 31064-34094 |

Knowledge of the PAV genome sequence is useful for both therapeutic and diagnostic procedures. Regions suitable for insertion and regulated expression of heterologous sequences have been identified. These regions include, but are not limited to the E1 region including E1A, E1B^{small} and E1B^{large}, E3 and E4 regions, and the region between the E4 region and the right end of the genome. A heterologous nucleotide sequence, with respect to the PAV vectors of the invention, is one which is not normally associated with PAV sequences as part of the PAV genome. Heterologous nucleotide sequences include synthetic sequences. Regions encoding immunogenic PAV polypeptides, for use in immunodiagnostic procedures, have also been identified and are disclosed herein. These include the regions encoding the following PAV proteins: E1A, E1B^{small} and E1B^{large}, E4, pIX, DBP, pTP, pol, IVa2, 52K, IIIA, pIII, pVII, pV, pX, pVI, 33K, pVIII, hexon and fiber (see Table 2). Regions essential for viral replication, such as E1 regions E1A and E1B^{large} and E2A, can be deleted to provide attenuated strains for use as vaccines. Nonessential regions, such as E1B^{small} and parts of the E3 and E4 regions, can be deleted to provide insertion sites, or to provide additional capacity for insertion at a site other than the deleted region. Deletions of viral sequences can be obtained by any method known in the art, including but not limited to restriction enzyme digestion and ligation, oligonucleotide-mediated deletion mutagenesis, and the like.

The practice of the present invention employs, unless otherwise indicated, conventional microbiology, immunology, virology, molecular biology, and recombinant DNA techniques which are within the skill of the art. These techniques are fully explained in the literature. See, e.g., Maniatis et al., Molecular Cloning: A Laboratory Manual (1982); DNA Cloning: A Practical Approach vols. I & II (D. Glover, ed.); Oligonucleotide Synthesis (N. Gait, ed. (1984)); Nucleic Acid Hybridization (B. Hames & S. Higgins, eds. (1985)); Transcription and Translation (B. Hames & S. Higgins, eds. (1984)); Animal Cell Culture (R. Freshney, ed. (1986)); Perbal, A Practical Guide to Molecular Cloning (1984); Ausubel, et al., Current Protocols In Molecular Biology, John Wiley & Sons (1987, 1988, 1989, 1990, 1991, 1992, 1993, 1994, 1995, 1996); and Sambrook et al., Molecular Cloning: A Laboratory Manual (2^{nd} Edition); vols. I, II & III (1989).

Nucleotide Sequence, Genome Organization, and Transcription Map of Porcine Adenovirus Type 3 (PAV-3).

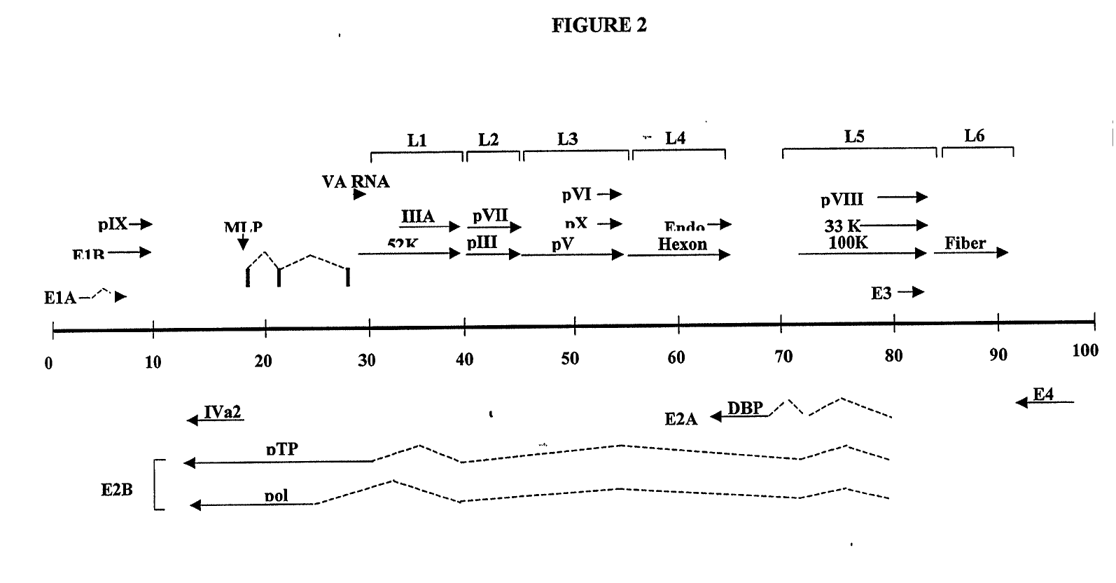

The complete nucleotide sequence of PAV-3 genome is 34,094 base pairs (bp) in length and has a base composition of 31.3% G, 32.5% C, 18.3% A, and 17.9% T. Thus, the sequence of the PAV-3 genome has a G+C content of 63.8%, which is unusually high when compared with the G+C content of many other animal adenoviruses. The genome termini share inverted terminal repeats (ITR) of 144 bp. Reddy et al., 1995c, supra. The organization of the genome as determined by analysis of open reading frames (ORFs), nuclease protection mapping, and sequencing of cDNA clones, is summarized in Table 2 and FIG. 2. The present invention relates to the characterization of the PAV E1 region. For PAV3, the E1A region is from nucleotide 533 to nucleotide 1222 of FIG. 1, the E1B^{small} region is from nucleotide 1461 to nucleotide 2069 of FIG. 1 and the E1B^{large} region is from nucleotide 1829 to nucleotide 3253 of FIG. 1. E1B^{small} and E1B^{large} nucleotide regions are overlapping and are differentially transcribed. Depending upon the intended use of the PAV vector, PAV constructs can be made comprising a deletion of part or all of the E1B^{small} region. For example, if the entire E1B function is intended to be deleted, the entire E1B nucleotide region from nucleotides 1461 to 3253 can be deleted; or the region from nucleotides 1461 to 2069 can be deleted (which disrupts both E1B^{small} and E1B^{large} function); or the region from 1461 to 2069 and additionally, any portion of nucleotides 2069 through 3253 can be deleted. If it is intended to delete E1B^{small} nucleotides while retaining E1B^{large} function, nucleotides 1461 to 1829 are deleted, leaving the nucleotide region for E1B^{large} intact.

One important feature of PAV-3 genome is the presence of a short virion associated (VA) RNA gene between the splice acceptor sites of the precursor terminal protein (pTP) and 52 kDa protein genes (FIG. 2). Expression of VA genes increases the kinetics of viral replication; thereby providing the potential for higher yields of recombinant gene products using the PAV vectors of the invention. The locations of the signature sequences present upstream and downstream of VA RNA genes indicate the VA RNA gene of PAV-3 is about 126 nucleotides (nt) in length. This is somewhat shorter than most VA RNAs, whose lengths are 163±14 nts, however shorter VA RNAs have also been reported in HAV-10 and CELO virus. Ma et al. (1996) J. Virol. 70:5083-5099; and Chiocca et al. (1996) J. Virol. 70:2939-2949. The VA RNA genes were not found in the genomes of BAV-3, CAV-1, and OAV. Reddy et al. (1998) J. Virol. 72:1394-1402; Morrison et al. (1997) J. Gen. Virol. 78:873-878; and Vrati et al. (1996) Virology 220:186-199.

In PAV-3 the major late transcript initiates at 17.7 map units (m.u.: an adenovirus map unit is 1% of genome length, starting from the left end of the genome). There are six 3′-coterminal families of late mRNAs, denoted L1 to L6 (see FIG. 2). All mRNAs produced from the major late promoter (MLP) contain a tripartite leader sequence (TPL). The first portion of the TPL lies next to the MLP and is 61 nts long. The second portion lies within the gene coding for pol and is 68 nt in length. The third portion is 99 nts long and is located within the gene coding for pTP. Thus the TPL of PAV-3 is 228 nt long and is derived from three exons located at 17.7, 20.9, and 28.1 m.u.

The MLP and TPL sequences can be used for expression of a heterologous sequence in a recombinant PAV vector or in any other adenoviral expression system.

TABLE 2
Transcriptional and Translational Features of the PAV-3 Genome

| | | Transcription | | | | Poly(A) | |
|---|---|---|---|---|---|---|---|
| Region | Gene | start site | ATG | Splice donor site | Splice acceptor site | signal | Poly(A) addition site |
| E1A | 229R | heterogeneous | 533 | | | 1286 | 1307 |
| | 214R | | 533 | 1043 | 1140 | 1286 | 1307 |
| E1B | 202R | 1382 | 1461 | | | 4085 | 4110,4112 |
| | 474R | 1382 | 1829 | | | 4085 | 4110,4112 |
| pIX | Pix | 3377 | 3394 | | | 4085 | 4110, 4112 |
| E2A | DBP | 17011c | 24041c | 26949c,24714c | 24793c,24051c | 22560c | 22536c |
| E2B | pTP | 17011c | 13638c | 24949c,24714c | 24793c,13772c | 4075c | 4053c |
| | pol | 17011c | 13638c | 24949c,24714c | 24793†c,13772†c | 4075c | 4053c |
| IVa2 | IVa2 | 5867c | 5711c | 5699c | 5441c | 4075c | 4053c |
| E3 | | 27473 | | | | 28765 | 28793 |
| E4 | | 33730c | | | | 31189c | 31170c |
| L1 | 52K | 6064 | 10629 | 9684 | 10606 | 13601 | 13627 |
| | IIIA | 6064 | 11719 | 9684 | 11715 | 13601 | 13627 |
| L2 | pIII | 6064 | 13662 | 9684 | 13662 | 15698* | 15735 |
| | pVII | 6064 | 15170 | 9684 | 15139 | 15698* | 15735 |
| L3 | pV | 6064 | 15819 | 9684 | 15793 | 18992 | 19013 |
| | pX | 6064 | 17783 | 9684 | 17776 | 18992 | 19013 |
| | pVI | 6064 | 18076 | 9684 | 18063 | 18992 | 19013 |
| L4 | Hexon | 6064 | 19097 | 9684 | 19096 | 22544 | 22567 |
| | Protease | 6064 | 21934 | 9684 | 21931† | 22544 | 22567 |
| L5 | 100k | 6064 | 24056 | 9684 | 24056 | 28765 | 28793 |
| | 33K | 6064 | 26181 | 9684 | 26130 | 28765 | 29793 |
| | pVIII | 6064 | 27089 | 9684 | 26792 | 28765 | 28793 |
| L6 | Fiber | 6064 | 28939 | 9684 | 28910 | 31143 | 31164 |

Notes:
*TTGTTT is present as a polyadenylation signal instead of AATAAA
†The splice acceptor sites for the pol and protease genes were determined based on consensus splice acceptor sequences
“c” refers to sequences on the complementary (leftward-reading) strand of the PAV genome.

Construction of Recombinant PAV Vectors

In one embodiment of the invention, a recombinant PAV vector is constructed by in vivo recombination between a plasmid and a PAV genome. Generally, heterologous sequences are inserted into a plasmid vector containing a portion of the PAV genome, which may or may not possess one or more deletions of PAV sequences. The heterologous sequences are inserted into the PAV insert portion of the plasmid vector, such that the heterologous sequences are flanked by PAV sequences that are adjacent on the PAV genome. The PAV sequences serve as “guide sequences,” to direct insertion of the heterologous sequences to a particular site in the PAV genome; the insertion site being defined by the genomic location of the guide sequences.

The vector is generally a bacterial plasmid, allowing multiple copies of the cloned sequence to be produced. In one embodiment, the plasmid is co-transfected, into an appropriate host cell, with a PAV genome comprising a full-length or nearly full-length PAV genomic sequence. The PAV genome can be isolated from PAV virions, or can comprise a PAV genome that has been inserted into a plasmid, using standard techniques of molecular biology and biotechnology. Construction of a plasmid containing a PAV genome is described in Example 2, infra. Nearly full-length PAV genomic sequences can be deleted in regions such as E1, E3, E4 and the region between E4 and the right end of the genome, but will retain sequences required for replication and packaging. PAV genomes can be deleted in essential regions, such as E1A and E1B^{large} if the essential function are supplied by a helper cell line.

Insertion of the cloned heterologous sequences into a viral genome occurs by in vivo recombination between a plasmid vector (containing heterologous sequences flanked by PAV guide sequences) and a PAV genome following co-transfection into a suitable host cell. The PAV genome contains inverted terminal repeat (ITR) sequences required for initiation of viral DNA replication (Reddy et al. (1995c), supra), and sequences involved in packaging of replicated viral genomes. Adenovirus packaging signals generally lie between the left ITR and the E1A promoter. Incorporation of the cloned heterologous sequences into the PAV genome thus places the heterologous sequences into a DNA molecule containing viral replication and packaging signals, allowing generation of multiple copies of a recombinant PAV genome that can be packaged into infectious viral particles. Alternatively, incorporation of the cloned heterologous sequences into a PAV genome places these sequences into a DNA molecule that can be replicated and packaged in an appropriate helper cell line. Multiple copies of a single sequence can be inserted to improve yield of the heterologous gene product, or multiple heterologous sequences can be inserted so that the recombinant virus is capable of expressing more than one heterologous gene product. The heterologous sequences can contain additions, deletions and/or substitutions to enhance the expression and/or immunological effect of the expressed gene product(s).

Attachment of guide sequences to a heterologous sequence can also be accomplished by ligation in vitro. In this case, a nucleic acid comprising a heterologous sequence flanked by PAV guide sequences can be co-introduced into a host cell along with a PAV genome, and recombination can occur to generate a recombinant PAV vector. Introduction of nucleic acids into cells can be achieved by any method known in the art, including, but not limited to, microinjection, transfection, electroporation, CaPO₄ precipitation, DEAE-dextran, liposomes, particle bombardment, etc.

In one embodiment of the invention, a recombinant PAV expression cassette can be obtained by cleaving a wild-type PAV genome with an appropriate restriction enzyme to produce a PAV restriction fragment representing, for example, the left end or the right end of the genome comprising E1 or E3 gene region sequences, respectively. The PAV restriction fragment can be inserted into a cloning vehicle, such as a plasmid, and thereafter at least one heterologous sequence (which may or may not encode a foreign protein) can be inserted into the E1 or E3 region with or without an operatively-linked eukaryotic transcriptional regulatory sequence. The recombinant expression cassette is contacted with a PAV genome and, through homologous recombination or other conventional genetic engineering methods, the desired recombinant is obtained. In the case wherein the expression cassette comprises the E1 essential regions, such as, E1A and/or E1B^{large} or some other essential region, recombination between the expression cassette and a PAV genome can occur within an appropriate helper cell line such as, for example, an E1A transformed cell line when E1A region is deleted or E1A function is inactivated. Restriction fragments of the PAV genome other than those comprising the E1 or E3 regions are also useful in the practice of the invention and can be inserted into a cloning vehicle such that heterologous sequences can be inserted into the PAV sequences. These DNA constructs can then undergo recombination in vitro or in vivo, with a PAV genome either before or after transformation or transfection of an appropriate host cell.

The invention also includes an expression system comprising a porcine adenovirus expression vector wherein a heterologous nucleotide sequence, e.g. DNA, replaces part or all of the E3 region, part or all of the E1 region, part or all of the E2 region, part or all of the E4 region, part or all of the late region and/or part or all of the regions occupied by the pIX, DBP, pTP, pol, IVa2, 52K, IIIA, pIII, pVII, pV, pX, pVI, and 33K genes. The expression system can be used wherein the foreign nucleotide sequences, e.g. DNA, are optionally in operative linkage with a eukaryotic transcriptional regulatory sequence. PAV expression vectors can also comprise inverted terminal repeat (ITR) sequences and packaging sequences.

The PAV E1A, E1B^{large}, pIX, DBP, pTP, pol, IVa2, 52K, IIIA, pIII, pVII, pV, pX, pVI, and 33K genes are essential for viral replication. Therefore, PAV vectors comprising deletions in any of these genes, or which lack functions encoded by any of these genes, are grown in an appropriate complementing cell line (i.e., a helper cell line). E1B^{Small} and most, if not all, of the open reading frames in the E3 and E4 regions of PAV-3 are non-essential for viral replication and, therefore, deletions in these regions can be constructed for insertion or to increase vector capacity, without necessitating the use of a helper cell line for growth of the viral vector.

In another embodiment, the invention provides a method for constructing a full-length clone of a PAV genome by homologous recombination in vivo. In this embodiment, two or more plasmid clones, containing overlapping segments of the PAV genome and together covering the entire genome, are introduced into an appropriate bacterial host cell. Approximately 30 base pairs of overlap is required for homologous recombination in E. coli. Chartier et al. (1996) J. Virol. 70:4805-4810. Through in vivo homologous recombination, the PAV genome segments are joined to form a full-length PAV genome. In a further embodiment, a recombinant plasmid containing left-end sequences and right-end sequences of the PAV genome, separated by a unique restriction site, is constructed. This plasmid is digested with the restriction enzyme recognizing the unique restriction site, to generate a unit-length linear plasmid, which is introduced into a cell together with a full-length PAV genome. Homologous recombination within the cell will result in production of a recombinant plasmid containing a full-length PAV genome. Recombinant plasmids will also generally contain sequences specifying replication in a host cell and one or more selective markers, such as, for example, antibiotic resistance.

Suitable host cells include any cell that will support recombination between a PAV genome and a plasmid containing PAV sequences, or between two or more plasmids, each containing PAV sequences. Recombination is generally performed in procaryotic cells, such as E. coli, while transfection of a plasmid containing a viral genome, to generate virus particles, is conducted in eukaryotic cells, preferably mammalian cells, most preferably porcine cell cultures. The growth of bacterial cell cultures, as well as culture and maintenance of eukaryotic cells and mammalian cell lines are procedures which are well-known to those of skill in the art.

In one embodiment of the invention, a replication-defective recombinant PAV vector is used for expression of heterologous sequences. In some embodiments, the replication-defective vector lacks E1A and/or E1B^{large} region function. In preferred embodiments, the replication-defective PAV vector comprises a deletion of the E1A region or an inactivation of the E1A gene function, such as through an insertion in the E1A gene region. Construction of a deletion in the E1 region of PAV is described in Example 3 and Example 10, infra. Heterologous sequences can be inserted so as to replace the deleted E1A or E1B region(s), and/or can be inserted at other sites in the PAV genome, preferably E3, E4 and/or the region between E4 and the right end of the genome. Replication-defective vectors with deletions in essential E1 regions, such as, E1A and E1B^{large} are grown in helper cell lines, which provide the deleted E1 function.

Accordingly, in one embodiment of the invention, a number of recombinant helper cell lines are produced according to the present invention by constructing an expression cassette comprising an adenoviral essential E1 region, such as E1A and E1B^{large} and transforming host cells therewith to provide complementing cell lines or cultures providing E1 deleted functions. In preferred embodiments, the host cell is transformed with a human E1A gene region. The terms “complementing cell,” “complementing cell line,” “helper cell” and “helper cell line” are used interchangeably herein to denote a cell line that provides a viral function that is deficient in a deleted PAV, preferably an essential E1 function. These recombinant complementing cell lines are capable of allowing a replication-defective recombinant PAV, having a deleted E1 gene region that is essential for replication, such as E1A and E1B^{large}, wherein the deleted sequences are optionally replaced by heterologous nucleotide sequences, to replicate and express one or more foreign genes or fragments thereof encoded by the heterologous nucleotide sequences. PAV vectors with E1 deletions, wherein heterologous sequences are inserted in regions other than E1, can also be propagated in these complementing cell lines, and will express the heterologous sequences if they are inserted downstream of a PAV promoter or are inserted in operative linkage with a eukaryotic regulatory sequence. Preferred helper cell lines include VIDO R1 cells, as described in Example 1, infra. Briefly, the VIDO R1 cell line is a porcine retinal cell line that has been transfected with DNA from the human adenovirus type 5 (HAV-5) E1 region, and which supports the growth of PAV E1A deletions and HAV-5 E1 deletions.

In the present invention, a PAV E1-complementing cell line employing the E1 region of HAV-5 is shown to complement PAV-3 E1 mutants. There are several reasons that the E1 region of HAV-5 was used for transformation of porcine embryonic retinal cells. The E1 region of HAV-5 was shown to transform human retina cells very efficiently. Fallaux et al. (1998) supra. The E1 region of HAV-5 has been thoroughly characterized and the monoclonal antibodies against the E1 proteins are readily available from commercial sources. In addition, the E1A region of HAV-5 was shown to complement the E1A functions of several non-human adenoviruses. Ball et al. (1988) J. Virol. 62:3947-3957; Zheng et al. (1994) Virus Res. 31:163-186.

More generally, replication-defective recombinant PAV vectors, lacking one or more essential functions encoded by the PAV genome, can be propagated in appropriate complementing cell lines, wherein a particular complementing cell line provides a function or functions that is (are) lacking in a particular defective recombinant PAV vector. Complementing cell lines can provide viral functions through, for example, co-infection with a helper virus, or by integrating or otherwise maintaining in stable form a fragment of a viral genome encoding a particular viral function.

In another embodiment of the invention, E1 function (or the function of any other viral region which may be mutated or deleted in any particular viral vector) can be supplied (to provide a complementing cell line) by co-infection of cells with a virus which expresses the function that the vector lacks.

PAV Expression Systems

In one embodiment, the present invention identifies and provides means of deleting regions of the PAV genome, to provide sites into which heterologous or homologous nucleotide sequences encoding foreign genes or fragments thereof can be inserted to generate porcine adenovirus recombinants. In preferred embodiments, deletions are made in part or all of the nucleotide sequences of the PAV E1, E3, or E4 regions and/or the region between E4 and the right end of genome. E1 gene region deletions are described in Example 3 and Example 10. E3 deletion and insertion of heterologous sequence in the E3 region are described in Example 4 and 5; and insertion of a heterologous sequence between the E4 region and the right end of the PAV genome, as well as expression of the inserted sequence, is described in Example 6, infra.

In another embodiment, the invention identifies and provides additional regions of the PAV genome (and fragments thereof) suitable for insertion of heterologous or homologous nucleotide sequences encoding foreign genes or fragments thereof to generate PAV recombinants. These regions include nucleotides 145-13,555; 15,284-19,035; 22,677-24,055; 26,573-27,088; and 31,149-34,094 and comprise the E2 region, the late region, and genes encoding the pIX, DBP, pTP, pol, IVa2, 52K, IIIA, pIII, pVII, pV, pX, pVI, and 33K proteins. These regions of the PAV genome can be used, among other things, for insertion of foreign sequences, for provision of DNA control sequences including transcriptional and translational regulatory sequences, or for diagnostic purposes to detect the presence, in a biological sample, of viral nucleic acids and/or proteins encoded by these regions. Example 7, infra, describes procedures for constructing insertions in these regions.

One or more heterologous sequences can be inserted into one or more regions of the PAV genome to generate a recombinant PAV vector, limited only by the insertion capacity of the PAV genome and ability of the recombinant PAV vector to express the inserted heterologous sequences. In general, adenovirus genomes can accept inserts of approximately 5% of genome length and remain capable of being packaged into virus particles. The insertion capacity can be increased by deletion of non-essential regions and/or deletion of essential regions whose function is provided by a helper cell line.

In one embodiment of the invention, insertion can be achieved by constructing a plasmid containing the region of the PAV genome into which insertion is desired. The plasmid is then digested with a restriction enzyme having a recognition sequence in the PAV portion of the plasmid, and a heterologous sequence is inserted at the site of restriction digestion. The plasmid, containing a portion of the PAV genome with an inserted heterologous sequence, in co-transformed, along with a plasmid (such as pPAV-200) containing a full-length PAV genome, into a bacterial cell (such as, for example, E. coli), wherein homologous recombination between the plasmids generates a full-length PAV genome containing inserted heterologous sequences.

Deletion of PAV sequences, to provide a site for insertion of heterologous sequences or to provide additional capacity for insertion at a different site, can be accomplished by methods well-known to those of skill in the art. For example, for PAV sequences cloned in a plasmid, digestion with one or more restriction enzymes (with at least one recognition sequence in the PAV insert) followed by ligation will, in some cases, result in deletion of sequences between the restriction enzyme recognition sites. Alternatively, digestion at a single restriction enzyme recognition site within the PAV insert, followed by exonuclease treatment, followed by ligation will result in deletion of PAV sequences adjacent to the restriction site. A plasmid containing one or more portions of the PAV genome with one or more deletions, constructed as described above, can be co-transfected into a bacterial cell along with a plasmid containing a full-length PAV genome to generate, by homologous recombination, a plasmid containing a PAV genome with a deletion at a specific site. PAV virions containing the deletion can then be obtained by transfection of mammalian cells (such as ST or VIDO R1 cells) with the plasmid containing a PAV genome with a deletion at a specific site.

Expression of an inserted sequence in a recombinant PAV vector will depend on the insertion site. Accordingly, preferred insertion sites are adjacent to and downstream (in the transcriptional sense) of PAV promoters. The transcriptional map of PAV, as disclosed herein, provides the locations of PAV promoters. Locations of restriction enzyme recognition sequences downstream of PAV promoters, for use as insertion sites, can be easily determined by one of skill in the art from the PAV nucleotide sequence provided herein. Alternatively, various in vitro techniques can be used for insertion of a restriction enzyme recognition sequence at a particular site, or for insertion of heterologous sequences at a site that does not contain a restriction enzyme recognition sequence. Such methods include, but are not limited to, oligonucleotide-mediated heteroduplex formation for insertion of one or more restriction enzyme recognition sequences (see, for example, Zoller et al. (1982) Nucleic Acids Res. 10:6487-6500; Brennan et al. (1990) Roux's Arch. Dev. Biol. 199:89-96; and Kunkel et al. (1987) Meth. Enzymology 154:367-382) and PCR-mediated methods for insertion of longer sequences. See, for example, Zheng et al. (1994) Virus Research 31:163-186.

It is also possible to obtain expression of a heterologous sequence inserted at a site that is not downstream from a PAV promoter, if the heterologous sequence additionally comprises transcriptional regulatory sequences that are active in eukaryotic cells. Such transcriptional regulatory sequences can include cellular promoters such as, for example, the bovine hsp70 promoter and viral promoters such as, for example, herpesvirus, adenovirus and papovavirus promoters and DNA copies of retroviral long terminal repeat (LTR) sequences.

In another embodiment, homologous recombination in a procaryotic cell can be used to generate a cloned PAV genome; and the cloned PAV-3 genome can be propagated as a plasmid. Infectious virus can be obtained by transfection of mammalian cells with the cloned PAV genome rescued from plasmid-containing cells. Example 2, infra describes construction of an infectious plasmid containing a PAV-3 genome.

The invention provides PAV regulatory sequences which can be used to regulate the expression of heterologous genes. A regulatory sequence can be, for example, a transcriptional regulatory sequence, a promoter, an enhancer, an upstream regulatory domain, a splicing signal, a polyadenylation signal, a transcriptional termination sequence, a translational regulatory sequence, a ribosome binding site and a translational termination sequence.

Therapeutic Genes and Polypeptides

The PAV vectors of the invention can be used for the expression of therapeutic polypeptides in applications such as in vitro polypeptide production, vaccine production, nucleic acid immunization and gene delivery, for example. Therapeutic polypeptides comprise any polypeptide sequence with therapeutic and/or diagnostic value and include, but are not limited to, coagulation factors, growth hormones, cytokines, lymphokines, tumor-suppressing polypeptides, cell receptors, ligands for cell receptors, protease inhibitors, antibodies, toxins, immunotoxins, dystrophins, cystic fibrosis transmembrane conductance regulator (CFTR) and immunogenic polypeptides.

In a preferred embodiment, PAV vectors will contain heterologous sequences encoding protective determinants of various pathogens of swine, for use in subunit vaccines and nucleic acid immunization. Representative swine pathogen antigens include, but are not limited to, pseudorabies virus (PRV) gp50; transmissible gastroenteritis virus (TGEV) S gene; porcine rotavirus VP7 and VP8 genes; genes of porcine respiratory and reproductive syndrome virus (PRRS), in particular ORFs 3, 4 and 5; genes of porcine epidemic diarrhea virus; genes of hog cholera virus, genes of porcine parvovirus, and genes of porcine influenza virus.

Various foreign genes or nucleotide sequences or coding sequences (prokaryotic, and eukaryotic) can be inserted into a PAV vector, in accordance with the present invention, particularly to provide protection against a wide range of diseases. Many such genes are already known in the art; the problem heretofore having been to provide a safe, convenient and effective vaccine vector for the genes or sequences.

A heterologous (i.e., foreign) nucleotide sequence can consist of one or more gene(s) of interest, and preferably of therapeutic interest. In the context of the present invention, a gene of interest can code either for an antisense RNA, a ribozyme or for an mRNA which will then be translated into a protein of interest. A gene of interest can be of genomic type, of complementary DNA (cDNA) type or of mixed type (minigene, in which at least one intron is deleted). It can code for a mature protein, a precursor of a mature protein, in particular a precursor intended to be secreted and accordingly comprising a signal peptide, a chimeric protein originating from the fusion of sequences of diverse origins, or a mutant of a natural protein displaying improved or modified biological properties. Such a mutant can be obtained by deletion, substitution and/or addition of one or more nucleotide(s) of the gene coding for the natural protein, or any other type of change in the sequence encoding the natural protein, such as, for example, transposition or inversion.

A gene of interest can be placed under the control of regulatory sequences suitable for its expression in a host cell. Suitable regulatory sequences are understood to mean the set of elements needed for transcription of a gene into RNA (ribozyme, antisense RNA or mRNA), for processing of RNA, and for the translation of an mRNA into protein. Among the elements needed for transcription, the promoter assumes special importance. It can be a constitutive promoter or a regulatable promoter, and can be isolated from any gene of eukaryotic, prokaryotic or viral origin, and even adenoviral origin. Alternatively, it can be the natural promoter of the gene of interest. Generally speaking, a promoter used in the present invention can be chosen to contain cell-specific regulatory sequences, or modified to contain such sequences. For example, a gene of interest for use in the present invention is placed under the control of an immunoglobulin gene promoter when it is desired to target its expression to lymphocytic host cells. There may also be mentioned the HSV-1 TK (herpesvirus type 1 thymidine kinase) gene promoter, the adenoviral MLP (major late promoter), in particular of human adenovirus type 2, the RSV (Rous Sarcoma Virus) LTR (long terminal repeat), the CMV (Cytomegalovirus) early promoter, and the PGK (phosphoglycerate kinase) gene promoter, for example, permitting expression in a large number of cell types.

Alternatively, targeting of a recombinant PAV vector to a particular cell type can be achieved by constructing recombinant hexon and/or fiber genes. The protein products of these genes are involved in host cell recognition; therefore, the genes can be modified to contain peptide sequences that will allow the virus to recognize alternative host cells.

Among genes of interest which are useful in the context of the present invention, there may be mentioned:

genes coding for cytokines such as interferons and interleukins;

genes encoding lymphokines;

genes coding for membrane receptors such as the receptors recognized by pathogenic organisms (viruses, bacteria or parasites), preferably by the HIV virus (human immunodeficiency virus);

genes coding for coagulation factors such as factor VIII and factor IX;

genes coding for dystrophins;

genes coding for insulin;

genes coding for proteins participating directly or indirectly in cellular ion channels, such as the CFTR (cystic fibrosis transmembrane conductance regulator) protein;

genes coding for antisense RNAs, or proteins capable of inhibiting the activity of a protein produced by a pathogenic gene which is present in the genome of a pathogenic organism, or proteins (or genes encoding them) capable of inhibiting the activity of a cellular gene whose expression is deregulated, for example an oncogene;

genes coding for a protein inhibiting an enzyme activity, such as α₁-antitrypsin or a viral protease inhibitor, for example;

genes coding for variants of pathogenic proteins which have been mutated so as to impair their biological function, such as, for example, trans-dominant variants of the tat protein of the HIV virus which are capable of competing with the natural protein for binding to the target sequence, thereby preventing the activation of HIV;

genes coding for antigenic epitopes in order to increase the host cell's immunity;

genes coding for major histocompatibility complex classes I and II proteins, as well as the genes coding for the proteins which are inducers of these genes;

genes coding for antibodies;

genes coding for immunotoxins;

genes encoding toxins;

genes encoding growth factors or growth hormones;

genes encoding cell receptors and their ligands;

genes encoding tumor suppressors;

genes coding for cellular enzymes or those produced by pathogenic organisms; and

suicide genes. The HSV-1 TK suicide gene may be mentioned as an example. This viral TK enzyme displays markedly greater affinity compared to the cellular TK enzyme for certain nucleoside analogues (such as acyclovir or gancyclovir). It converts them to monophosphorylated molecules, which can themselves be converted by cellular enzymes to nucleotide precursors, which are toxic. These nucleotide analogues can be incorporated into replicating DNA molecules, hence incorporation occurs chiefly in the DNA of dividing cells. This incorporation can result in specific destruction of dividing cells such as cancer cells.

This list is not restrictive, and any other gene of interest can be used in the context of the present invention. In some cases the gene for a particular antigen can contain a large number of introns or can be from an RNA virus, in these cases a complementary DNA copy (cDNA) can be used. It is also possible that only fragments of nucleotide sequences of genes can be used (where these are sufficient to generate a protective immune response or a specific biological effect) rather than the complete sequence as found in the wild-type organism. Where available, synthetic genes or fragments thereof can also be used. However, the present invention can be used with a wide variety of genes, fragments and the like, and is not limited to those set out above.

Recombinant PAV vectors can be used to express antigens for provision of, for example, subunit vaccines. Antigens used in the present invention can be either native or recombinant antigenic polypeptides or fragments. They can be partial sequences, full-length sequences, or even fusions (e.g., having appropriate leader sequences for the recombinant host, or with an additional antigen sequence for another pathogen). The preferred antigenic polypeptide to be expressed by the virus systems of the present invention contain full-length (or near full-length) sequences encoding antigens. Alternatively, shorter sequences that are antigenic (i.e., encode one or more epitopes) can be used. The shorter sequence can encode a “neutralizing epitope,” which is defined as an epitope capable of eliciting antibodies that neutralize virus infectivity in an in vitro assay. Preferably the peptide should encode a “protective epitope” that is capable of raising in the host a “protective immune response;” i.e., a humoral (i.e. antibody-mediated), cell-mediated, and/or mucosal immune response that protects an immunized host from infection.

The antigens used in the present invention, particularly when comprised of short oligopeptides, can be conjugated to a vaccine carrier. Vaccine carriers are well known in the art: for example, bovine serum albumin (BSA), human serum albumin (HSA) and keyhole limpet hemocyanin (KLH). A preferred carrier protein, rotavirus VP6, is disclosed in EPO Pub. No. 0259149, the disclosure of which is incorporated by reference herein.

Genes for desired antigens or coding sequences thereof which can be inserted include those of organisms which cause disease in mammals, particularly porcine pathogens such as pseudorabies virus (PRV), transmissible gastroenteritis virus (TGEV), porcine rotavirus, porcine respiratory and reproductive syndrome virus (PRRS), porcine epidemic diarrhea virus (PEDV), hog cholera virus (HCV), porcine parvovirus and the like. Genes encoding antigens of human pathogens are also useful in the practice of the invention.

Therapeutic Applications

With the recombinant viruses of the present invention, it is possible to elicit an immune response against disease antigens and/or provide protection against a wide variety of diseases affecting swine, cattle, humans and other mammals. Any of the recombinant antigenic determinants or recombinant live viruses of the invention can be formulated and used in substantially the same manner as described for the antigenic determinant vaccines or live vaccine vectors.

The present invention also includes pharmaceutical compositions comprising a therapeutically effective amount of a recombinant vector, recombinant virus or recombinant protein, prepared according to the methods of the invention, in combination with a pharmaceutically acceptable vehicle and/or an adjuvant. Such a pharmaceutical composition can be prepared and dosages determined according to techniques that are well-known in the art. The pharmaceutical compositions of the invention can be administered by any known administration route including, but not limited to, systemically (for example, intravenously, intratracheally, intraperitoneally, intranasally, parenterally, enterically, intramuscularly, subcutaneously, intratumorally or intracranially) or by aerosolization or intrapulmonary instillation. Administration can take place in a single dose or in doses repeated one or more times after certain time intervals. The appropriate administration route and dosage will vary in accordance with the situation (for example, the individual being treated, the disorder to be treated or the gene or polypeptide of interest), but can be determined by one of skill in the art.

The vaccines of the invention carrying foreign genes or fragments can be orally administered in a suitable oral carrier, such as in an enteric-coated dosage form. Oral formulations include such normally-employed excipients as, for example, pharmaceutical grades of mannitol, lactose, starch, magnesium stearate, sodium saccharin cellulose, magnesium carbonate, and the like. Oral vaccine compositions may be taken in the form of solutions, suspensions, tablets, pills, capsules, sustained release formulations, or powders, containing from about 10% to about 95% of the active ingredient, preferably about 25% to about 70%. An oral vaccine may be preferable to raise mucosal immunity (which plays an important role in protection against pathogens infecting the gastrointestinal tract) in combination with systemic immunity.

In addition, the vaccine can be formulated into a suppository. For suppositories, the vaccine composition will include traditional binders and carriers, such as polyalkaline glycols or triglycerides. Such suppositories may be formed from mixtures containing the active ingredient in the range of about 0.5% to about 10% (w/w), preferably about 1% to about 2%.

Protocols for administering to animals the vaccine composition(s) of the present invention are within the skill of the art in view of the present disclosure. Those skilled in the art will select a concentration of the vaccine composition in a dose effective to elicit antibody, cell-mediated and/or mucosal immune responses to the antigenic fragment. Within wide limits, the dosage is not believed to be critical. Typically, the vaccine composition is administered in a manner which will deliver between about 1 to about 1,000 micrograms of the subunit antigen in a convenient volume of vehicle, e.g., about 1-10 ml. Preferably, the dosage in a single immunization will deliver from about 1 to about 500 micrograms of subunit antigen, more preferably about 5-10 to about 100-200 micrograms (e.g., 5-200 micrograms).

The timing of administration may also be important. For example, a primary inoculation preferably may be followed by subsequent booster inoculations, for example, several weeks to several months after the initial immunization, if needed. To insure sustained high levels of protection against disease, it may be helpful to readminister booster immunizations at regular intervals, for example once every several years. Alternatively, an initial dose may be administered orally followed by later inoculations, or vice versa. Preferred vaccination protocols can be established through routine vaccination protocol experiments.

The dosage for all routes of administration of in vivo recombinant virus vaccine depends on various factors including, the size of patient, nature of infection against which protection is needed, carrier and the like and can readily be determined by those of skill in the art. By way of non-limiting example, a dosage of between approximately 10³ pfu and 108 pfu can be used. As with in vitro subunit vaccines, additional dosages can be given as determined by the clinical factors involved.

A problem that has beset the use of adenovirus vectors for immunization and gene delivery in humans is the rapid development of an immunological response (or indeed in some cases existing immunity) to human adenoviruses (HAVs). Recombinant PAV vectors are likely to be less immunogenic in humans and, for this and other reasons, will be useful either as a substitute for HAV vectors or in combination with HAV vectors. For example, an initial immunization with a HAV vector can be followed by booster immunizations using PAV vectors; alternatively, initial immunization with a recombinant PAV vector can be followed by booster immunizations with HAV and/or PAV vectors.

The presence of low levels of helper-independent vectors in the batches of helper-dependent human adenoviruses that are grown in complementing human cell lines has been reported. Fallaux et al. (1998) supra. This occurs as a result of recombination events between the viral DNA and the integrated adenoviral sequences present in the complementing cell line. Hehir et al. (1996) J. Virol. 70:8459-8467. This type of contamination constitutes a safety risk, which could result in the replication and spread of the virus. Complete elimination of helper-dependent adenoviruses in the batches of helper-dependent vectors can be achieved using two approaches. The first is by developing new helper cell lines and matched vectors that do not share any common sequences. Fallaux et al. (1998) supra. The second approach is to take advantage of possible cross-complementation between two distantly related adenoviruses such as HAV-5 and PAV-3. VIDO R1 cells contain the E1 coding sequences of HAV-5. Although there is no significant homology between the E1 regions of HAV-5 and PAV-3 at the nucleotide sequence level, the proteins produced from the region can complement each others' function(s). Thus, the problem of helper-independent vector generation by homologous recombination is eliminated when VIDO R1 cells are used for the propagation of recombinant PAV-3.

The invention also encompasses a method of treatment, according to which a therapeutically effective amount of a PAV vector, recombinant PAV, or host cell of the invention is administered to a mammalian subject requiring treatment. The finding that PAV-3 was effective in entering canine, sheep and bovine cells in which it does not replicate or replicates poorly is an important observation. See Example 8, infra. This may have implications in designing PAV-3 vectors for vaccination in these and other animal species.

PAV Expression Systems

Recombinant PAV vectors can be used for regulated expression of foreign polypeptides encoded by heterologous nucleotide sequences. Standard conditions of cell culture, such as are known to those of skill in the art, will allow maximal expression of recombinant polypeptides . They can be used, in addition, for regulated expression of RNAs encoded by heterologous nucleotide sequences, as in, for example, antisense applications and expression of ribozymes.

When the heterologous sequences encode an antigenic polypeptide, PAV vectors comprising insertions of heterologous nucleotide sequences can be used to provide large quantities of antigen which are useful, in turn, for the preparation of antibodies. Methods for preparation of antibodies are well-known to those of skill in the art. Briefly, an animal (such as a rabbit) is given an initial subcutaneous injection of antigen plus Freund's complete adjuvant. One to two subsequent injections of antigen plus Freund's incomplete adjuvant are given at approximately 3 week intervals. Approximately 10 days after the final injection, serum is collected and tested for the presence of specific antibody by ELISA, Western Blot, immunoprecipitation, or any other immunological assay known to one of skill in the art.

Adenovirus E1 gene products transactivate many cellular genes; therefore, cell lines which constitutively express E1 proteins can express cellular polypeptides at a higher levels than other cell lines. The recombinant mammalian, particularly porcine, cell lines of the invention can be used to prepare and isolate polypeptides, including those such as (a) proteins associated with adenovirus E1A proteins: e.g. p300, retinoblastoma (Rb) protein, cyclins, kinases and the like; (b) proteins associated with adenovirus E1B protein: e.g. p53 and the like; growth factors, such as epidermal growth factor (EGF), transforming growth factor (TGF) and the like; (d) receptors such as epidermal growth factor receptor (EGF-R), fibroblast growth factor receptor (FGF-R), tumor necrosis factor receptor (TNF-R), insulin-like growth factor receptor (IGF-R), major histocompatibility complex class I receptor and the like; (e) proteins encoded by proto-oncogenes such as protein kinases (tyrosine-specific protein kinases and protein kinases specific for serine or threonine), p21 proteins (guanine nucleotide-binding proteins with GTPase activity) and the like; (f) other cellular proteins such as actins, collagens, fibronectins, integrins, phosphoproteins, proteoglycans, histones and the like, and (g) proteins involved in regulation of transcription such as TATA-box-binding protein (TBP), TBP-associated factors (TAFs), Sp1 binding protein and the like.

Gene Delivery

The invention also includes a method for delivering a gene to a mammal, such as a porcine, human or other mammal in need thereof, to control a gene deficiency. In one embodiment, the method comprises administering to said mammal a live recombinant porcine adenovirus containing a heterologous nucleotide sequence encoding a non-defective form of said gene under conditions wherein the recombinant virus vector genome is incorporated into said mammalian genome or is maintained independently and extrachromosomally to provide expression of the required gene in the target organ or tissue. These kinds of techniques are currently being used by those of skill in the art to replace a defective gene or portion thereof. Examples of foreign genes, heterologous nucleotide sequences, or portions thereof that can be incorporated for use in gene therapy include, but are not limited to, cystic fibrosis transmembrane conductance regulator gene, human minidystrophin gene, alpha-1-antitrypsin gene and the like.

In particular, the practice of the present invention in regard to gene delivery in humans is intended for the prevention or treatment of diseases including, but not limited to, genetic diseases (for example, hemophilia, thalassemias, emphysema, Gaucher's disease, cystic fibrosis, Duchenne muscular dystrophy, Duchenne's or Becker's myopathy, etc.), cancers, viral diseases (for example, AIDS, herpesvirus infection, cytomegalovirus infection and papillomavirus infection) and the like. For the purposes of the present invention, the vectors, cells and viral particles prepared by the methods of the invention may be introduced into a subject either ex vivo, (i.e., in a cell or cells removed from the patient) or directly in vivo into the body to be treated. Preferably, the host cell is a human cell and, more preferably, is a lung, fibroblast, muscle, liver or lymphocytic cell or a cell of the hematopoietic lineage.

Diagnostic Applications

The PAV genome, or any subregion of the PAV genome, is suitable for use as a nucleic acid probe, to test for the presence of PAV nucleic acid in a subject or a biological sample. The presence of viral nucleic acids can be detected by techniques known to one of skill in the art including, but not limited to, hybridization assays, polymerase chain reaction, and other types of amplification reactions. Suitable labels and hybridization techniques are well-known to those of skill in the art. See, for example, Kessler (ed.), Nonradioactive Labeling and Detection of Biomolecules, Springer-Verlag, Berlin, 1992; Kricka (ed.) Nonisotopic DNA Probe Techniques, Academic Press, San Diego, 1992; Howard (ed.) Methods in Nonradioactive Detection, Appleton & Lange, Norwalk, 1993; Ausubel et al., supra; and Sambrook et al., supra. Diagnostic kits comprising the nucleotide sequences of the invention can also contain reagents for cell disruption and nucleic acid purification, as well as buffers and solvents for the formation, selection and detection of hybrids.

Regions of the PAV genome can be inserted into any expression vector known in the art and expressed to provide, for example, vaccine formulations, protein for immunization, etc. The amino acid sequence of any PAV protein can be determined by one of skill in the art from the nucleotide sequences disclosed herein. PAV proteins can be used for diagnostic purposes, for example, to detect the presence of PAV antigens. Methods for detection of proteins are well-known to those of skill in the art and include, but are not limited to, various types of direct and competitive immunoassays, ELISA, Western blotting, enzymatic assay, immunohistochemistry, etc. See, for example, Harlow & Lane (eds.): Antibodies, A Laboratory Manual, Cold Spring Harbor Press, New York, 1988. Diagnostic kits comprising PAV polypeptides or amino acid sequences can also comprise reagents for protein isolation and for the formation, isolation, purification and/or detection of immune complexes.

### EXAMPLES

Methods

Virus and Viral DNA.

The 6618 strain of PAV-3 was propagated in the swine testis (ST) cell line and in E1-transformed porcine retinal cells (VIDO R1, see below). Porcine embryonic retinal cells were obtained from the eyeballs of piglets delivered by caesarian section two weeks before the parturition date. Uninfected cells were grown in MEM supplemented with 10% fetal bovine serum (FBS). MEM with 2% FBS was used for maintenance of infected cells. Viral DNA was extracted either from infected cell monolayers by the method of Hirt (1967) J. Mol. Biol. 26:365-369, or from purified virions as described by Graham et al. (1991) in “Methods in Molecular Biology” Vol. 7, Gene transfer and expression protocols, ed. E. J. Murray, Humana Press, Clifton, N.J., pp. 109-128.

Plasmids and Genomic DNA Sequencing.

Selected restriction enzyme fragments of PAV-3 DNA were cloned into pGEM-3Z and pGEM-7Zf(+) plasmids (Promega). Nucleotide sequences were determined on both strands of the genome by the dideoxy chain-termination method using Sequenase® enzyme (U.S. Biochemicals) and the dye-terminator method with an Applied Biosystems (Foster City, Calif.) DNA sequencer.

cDNA Library.

A cDNA library was generated from polyadenylated RNA extracted from PAV-3 infected ST cells at 12 h and 24 h post infection. Double stranded cDNAs were made with reagents from Stratagene and cloned into Lambda ZAP vector. Plaques which hybridized to specific restriction enzyme fragments of PAV-3 DNA were plaque purified twice. Plasmids containing cDNAs were excised from the Lambda ZAP vector according to the manufacturer's protocol. The resulting plasmid clones were characterized by restriction endonuclease analysis and by sequencing of both ends of the cDNA insert with T3- and T7-specific primers. Selected clones were sequenced with internal primers. cDNA sequences were aligned with genomic sequences to determine the transcription map.

Viral Transcript Mapping by Nuclease Protection

Transcript mapping was conducted according to the method of Berk et al. (1977) Cell 12:721-732.

### Example 1

### Development of an E1-Complementing Helper Cell Line (VIDO R1)

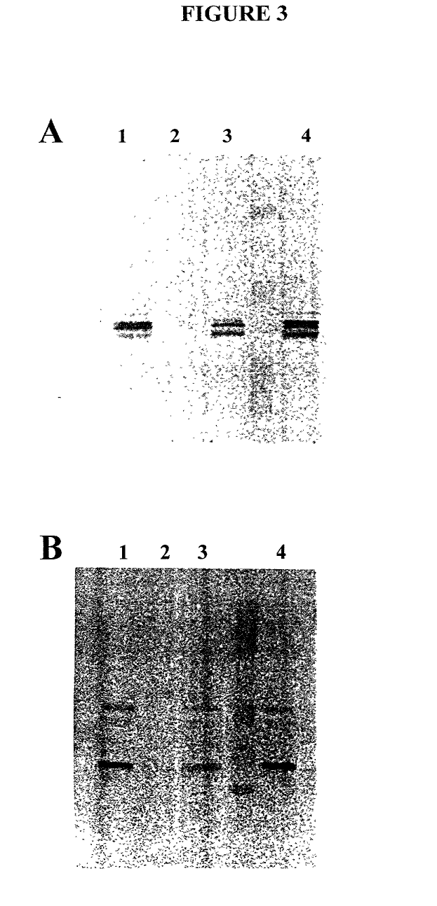

Primary cultures of porcine embryonic retina cells were transfected with 10 μg of plasmid pTG 4671 (Transgene, Strasbourg, France) by the calcium phosphate technique. The pTG 4671 plasmid contains the entire E1A and E1B sequences (nts 505-4034) of HAV-5, along with the puromycin acetyltransferase gene as a selectable marker. In this plasmid, the E1 region is under the control of the constitutive promoter from the mouse phosphoglycerate kinase gene, and the puromycin acetyltransferase gene is controlled by the constitutive SV40 early promoter. Transformed cells were selected by three passages in medium containing 7 μg/ml puromycin, identified based on change in their morphology from single foci (i.e., loss of contact inhibition), and subjected to single cell cloning. The established cell line was first tested for its ability to support the growth of E1 deletion mutants of HAV-5. Subsequently the cell line was further investigated for the presence of E1 sequences in the genome by PCR, expression of the E1A and E1B proteins by Western blot, and doubling time under cell culture conditions. E1 sequences were detected, and production of E1A and E1B proteins was demonstrated by immunoprecipitation (FIG. 3). Doubling time was shorter, when compared to that of the parent cell line. Example 3, infra, shows that this cell line is capable of complementing a PAV E1A deletion mutant.

To assess the stability of E1 expression, VIDO R1 cells were cultured through more than 50 passages (split 1:3 twice weekly) and tested for their ability to support the replication of E1-deleted HAV-5. Expression of the E1A and E1B proteins at regular intervals was also monitored by Western blot. The results indicated that the VIDO R1 line retained the ability to support the growth of E1-deleted virus and expressed similar levels of E1 proteins during more than 50 passages in culture. Therefore, VIDO R1 can be considered to be an established cell line.

### Example 2

### Construction of a Full-Length Infectious Clone of PAV-3

A plasmid clone containing a full-length copy of the PAV-3 genome (pPAV-200) was generated by first constructing a plasmid containing left- and right-end sequences of PAV-3, with the PAV-3 sequences bordered by PacI sites and separated by a PstI restriction site (pPAV-100), then allowing recombination between PstI-digested pPAV-100 and an intact PAV-3 genome. Left- and right-end sequences for insertion into pPAV-100 were produced by PCR amplification, as follows.

The plasmid p3SB (Reddy et al., 1993, Intervirology 36:161-168), containing the left end of PAV-3 genome (position 1-8870) was used for amplification of the first 433 bp of the PAV-3 genome by PCR.

Amplification primers were oligonucleotides 1

| (5′-GCGGATCCTTAATTAACATCATCAATAATATA | (SEQ ID NO.:2) |
|---|---|
| CCGCACACTTTT-3′) | |
| and 2 | |
| (5′-CACCTGCAGATACACCCACACACGTCATCTCG | (SEQ ID NO.:3) |
| -3′). | |

In the sequences shown here, adenoviral sequences are shown in bold/underlined and engineered restriction enzyme sites are italicized.

For amplification of sequences at the right end of the PAV-3 genome, the plasmid p3SA (Reddy et al., 1993, supra) was used. This plasmid was used as template in PCR for amplification of the terminal 573 bp of the genome using oligonucleotide 1 (above) and oligonucleotide 3 (5′-CACCTGCAGCCTCCTGAGTGTGAAGAGTGTCC-3′) (SEQ ID NO.: 4). The primers were designed based on the nucleotide sequence information described elsewhere (Reddy et al., 1995c, supra; and Reddy et al., 1997, supra).

For construction of pPAV-100, the PCR product obtained with oligonucleotides 1 and 2 was digested with BamHI and PstI restriction enzymes and the PCR product obtained using primers 1 and 3 was digested with PstI and PacI enzymes. Modified bacterial plasmid pPolyIIsn14 was digested with BamHI and PacI enzymes. This plasmid was used based on its suitability for homologous recombination in E. coli. The two PCR products described above were cloned into pPolyIIsn14 by three way ligation to generate the plasmid pPAV-100 which carries both termini of PAV-3, separated by a PstI site and bordered by PacI restriction enzyme sites.

Plasmid pPAV-200, which contains a full length PAV-3 genome, was generated by co-transformation of E. coli BJ 5183 recBC sbcBC (Hanahan, 1983, J. Mol. Biol. 166:557-580) with PstI-linearized pPAV-100 and the genomic DNA of PAV-3. Extensive restriction enzyme analysis of pPAV-200 indicated that it had the structure expected of a full-length PAV-3 insert, and that no unexpected rearrangements had occurred during recombination in E. coli.

The infectivity of pPAV-200 was demonstrated by lipofectin transfection (Life Technologies, Gaithersburg, Md.) of ST cells following PacI enzyme digestion of the plasmid to release the viral genome from the plasmid. Viral plaques were evident 7 days following transfection, and titers were equivalent to, or higher than, those obtained after infection with wild-type PAV. The plaques were amplified and the viral DNA was extracted and analyzed by restriction enzyme digestion. The viral DNA obtained by cleavage of pPAV-200 with PacI contained an extra 3 bases at each end; but these extra bases did not substantially reduce the infectivity of the PAV genome excised from pPAV-200. In addition, the bacterial-derived genomes lacked the 55-kDa terminal protein that is covalently linked to the 5′ ends of adenoviral DNAs and which enhances infectivity of viral DNA.

### Example 3

### Generation of E1 Deletion Mutants of PAV-3

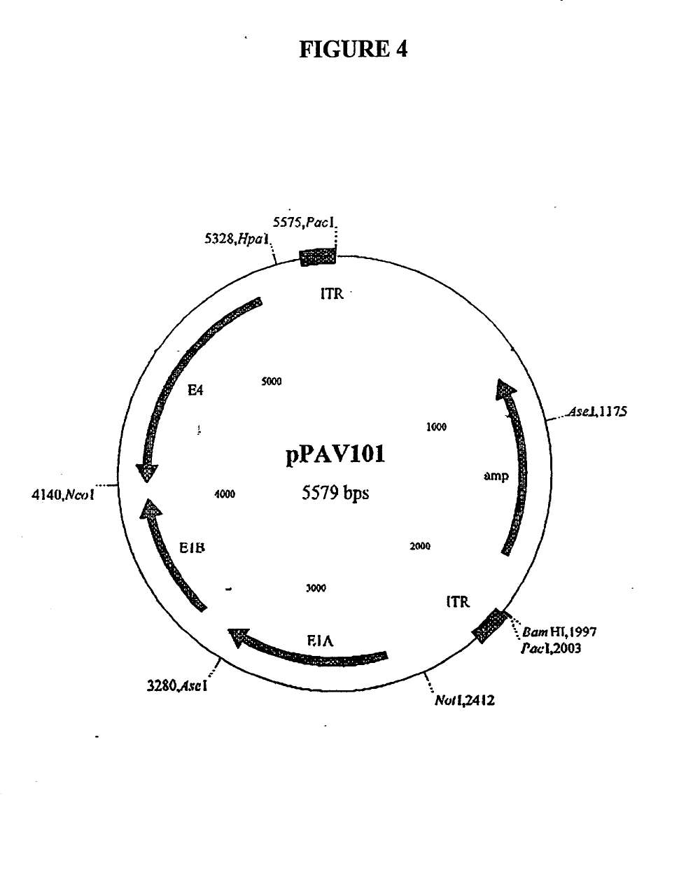

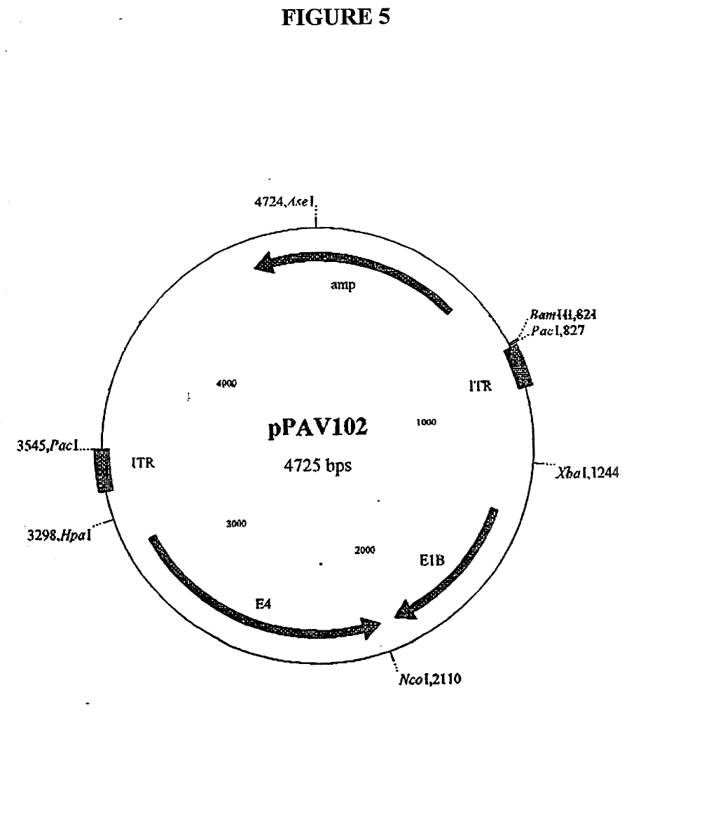

A plasmid (pPAV-101) containing the left (nucleotides 1-2, 130) and the right (nucleotides 32,660-34,094) terminal NcoI fragments of the PAV-3 genome was constructed by digesting pPAV-200 with the enzyme NcoI (which has no recognition sites in the vector backbone, but many sites in the PAV insert), gel-purifying the appropriate fragment and self-ligating the ends. See FIG. 4. The E1A sequences of pPAV-101, between nucleotides 407 and 1270 (PAV genome numbering), were deleted by digestion of pPAV-101 with NotI (recognition site at nucleotide 407) and AseI (recognition site at 1270), generation of blunt ends, and insertion of a double-stranded oligonucleotide encoding a XbaI restriction site to create a plasmid, pPAV-102, containing PAV left- and right-end sequences, separated by a NcoI site, with a deletion of the E1A region and a XbaI site at the site of the deletion. See FIG. 5. Plasmid pPAV-201, containing a full-length PAV-3 genome minus E1A sequences, was created by co-transformation of E. coli BJ 5183 with NcoI linearized pPAV-102 and genomic PAV-3 DNA. The resulting construct, when transfected into VIDO R1 cells following digestion with PacI restriction enzyme, produced a virus that had a deletion in the E1 region. In similar fashion, construction of a virus with deletions in E1 and E3 was accomplished by transformation of BJ 5183 cells with NcoI linearized pPAV-102 and genomic PAV-3 DNA containing an E3 deletion. These E1A deletion mutants did not grow on either ST (swine testis) cells or fetal porcine retina cells and could only be grown in the VIDO R1 cell line.

### Example 4

### Generation of E3 Inserts and Deletion Mutants

To systematically examine the extent of the E3 region that could be deleted, a E3 transfer vector was constructed. The vector (pPAV-301) contained a PAV-3 segment from nucleotides 26,716 to 31,064 with a green fluorescent protein (GFP) gene inserted into the SnaBI site (located at nucleotide 28,702) in the same orientation as E3. The GFP gene was obtained from the plasmid pGreen Lantern-1™ (Life Technologies), by NotI digestion followed by purification of a 732-nucleotide fragment. Similarly, another construct was made with GFP cloned into the SacI site located at nucleotide 27,789. KpnI-BamHI fragments encompassing the modified E3 regions were then isolated from these E3 transfer vectors and recombined in E coli with pPAV-200 that had been linearized at nucleotide position 28,702 by SnaBI digestion. Virus were obtained with a construct that had the GFP gene cloned into the SnaBI site.

To delete the non-essential portion of E3 from the transfer vector, a PCR approach was used. In this approach, the region of the PAV genome between nucleotides 27,402 and 28,112 was amplified using the following primers:

| | |
|---|---|
| 5′-GACTGACGCCGGCATGCAAT-3′ | SEQ ID NO:5 |
| 5′-CGGATCCTGACGCTACGAGCGGTTGTA-3′ | SEQ ID NO:6 |

In a second PCR reaction, the portion of the PAV genome between nucleotides 28,709 and 29,859 was amplified using the following two primers:

| | |
|---|---|
| 5′-CGGATCCATACGTACAGATGAAGTAGC-3′ | SEQ ID NO:7 |
| 5′-TCTGACTGAAGCCGACCTGC-3′ | SEQ ID NO:8 |

In the oligonucleotides designated SEQ ID NO: 6 and SEQ ID NO: 7, a BamHI recognition sequence is indicated by underlining. The template for amplification was a KpnI-BamHI fragment encompassing nucleotides 26,716-31,063 of the PAV genome, inserted into the plasmid pGEM3Z (Promega), and Pfu polymerase (Stratagene) was used for amplification. The first PCR product (product of amplification with SEQ ID NO: 5 and SEQ ID NO: 6) was digested with BamHI and gel- purified. The second PCR product (product of amplification with SEQ ID NO: 7 and SEQ ID NO: 8) was digested with BamHI and SpeI and gel-purified. They were inserted into SmaI/SpeI-digested pBlueScript II SK(+) (Stratagene) in a three-way ligation reaction to generate pPAV-300. See FIG. 6. pPAV-300 contains the portion of the PAV-3 genome extending from nucleotides 27,402 to 29,859, with 594 base pairs (bp) between nucleotides 28,113 and 28,707 deleted from the E3 region. A virus with such a deletion was constructed as follows. A SphI-SpeI fragment from pPAV-300, containing part of the pVIII gene, a deleted E3 region, and part of the fiber gene was isolated (see FIG. 6). This fragment was co-transfected, with SnaBI-digested pPAV-200 (which contains a full-length PAV-3 genome) into E. coli. Homologous recombination generated a plasmid, pFPAV-300, containing a full-length PAV genome with a deletion in the E3 region. pFPAV-300 was digested with PacI and transfected into VIDO R1 cells (Example 1) to generate recombinant virus with a deletion in the E3 region of the genome.

### Example 5

### Construction of a PAV Recombinant with an Insertion of the PRV gp50 Gene in the PAV E3 Region and Expression of the Inserted Gene

To construct a recombinant PAV expressing pseudorabies virus (PRV) gp50, the PRV gp50 gene was inserted at the SnaBI site of pPAV-300 to create plasmid pPAV-300-gp50. A SphI-SpeI fragment from pPAV-300-gp50, containing part of the pVII gene, a deleted E3 region with the PRV gp50 gene inserted, and part of the fiber gene, was purified and co-transfected, along with SnaBI-digested pFPAV-300 (E3-deleted) into E. coli. In the bacterial cell, homologous recombination generated pFPAV-300-gp50, a plasmid containing a PAV genome with the PRV gp50 gene replacing a deleted E3 region. Recombinant virus particles were obtained as described in Example 4.

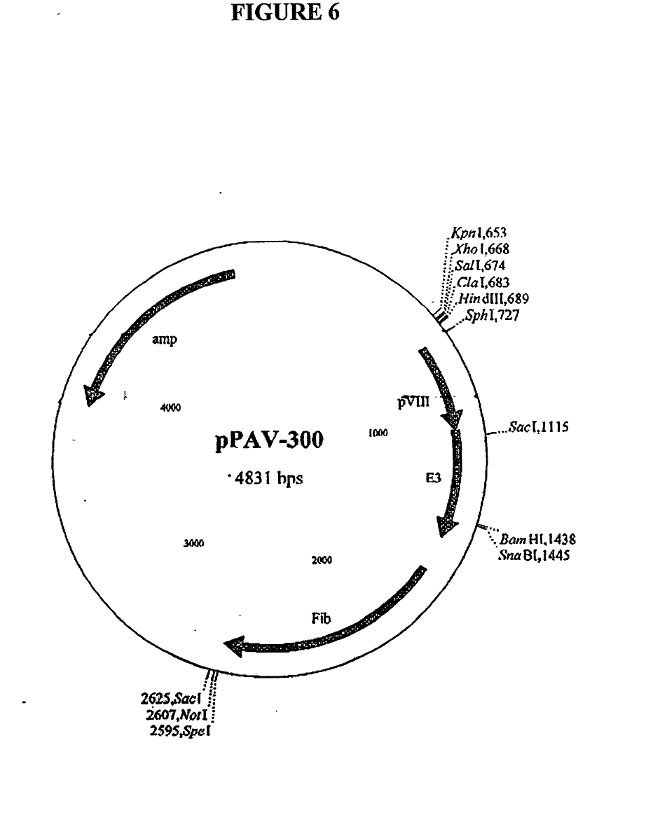

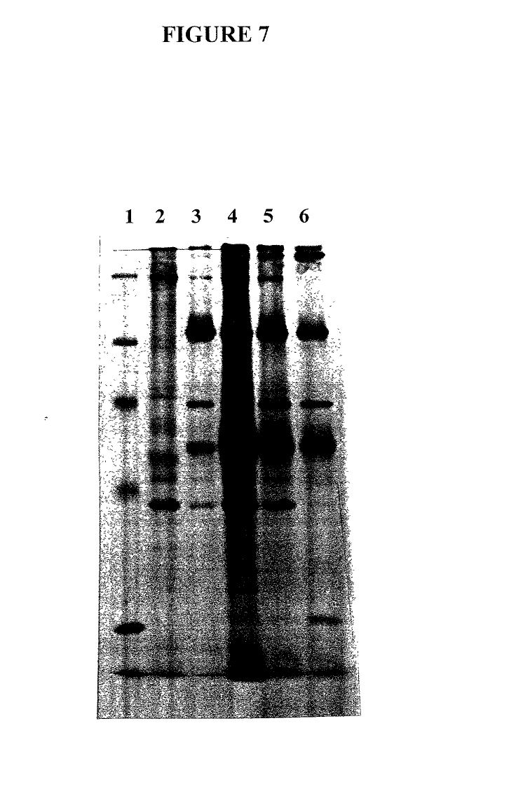

Expression of the inserted PRV gp50 was tested after infection of VIDO R1 cells with the recombinant virus, by ³⁵S labeling of infected cells (continuous label), followed by immunoprecipitation with an anti-gp50 monoclonal antibody and gel electrophoresis of the immunoprecipitate. FIG. 7 shows that large amounts of gp50 are present by 12 hours after infection, and expression of gp50 persists up to 24 hours after infection.

### Example 6

### Expression of the Chloramphenicol Acetyltransferase Gene from a Region that Lies Between the Promoter of the E4 Region and the Right ITR

The right terminal fragment of the PAV genome (encompassing nucleotides 31,054-34,094) was obtained by XhoI digestion of pPAV-200 and cloned between the XhoI and NotI sites ofpPolyllsnl4. A Chloramphenicol acetyltransferase (CAT) gene expression cassette, in which the CAT gene was flanked by the SV40 early promoter and the SV40 polyadenylation signal, was inserted, in both orientations, into a unique HpaI site located between the E4 region promoter and the right ITR, to generate plasmids pPAV-400A and pPAV-400B. The modified terminal fragments were transferred into a plasmid containing a full-length PAV-3 genome by homologous recombination in E coli between the isolated terminal fragments and HpaI-digested pPAV-200. Recombinant viruses expressing CAT were obtained following transfection of VIDO R1 cells with the plasmids. PAV-CAT2 contained the CAT gene cassette in a leftward transcriptional orientation (i.e., the same orientation as E4 region transcription), while, in PAV-CAT6, the CAT gene cassette was in the rightward transcriptional orientation.

These recombinant viruses were tested for expression of CAT, after infection of VIDO R1 cells, using a CAT Enzyme Assay System from Promega, following the instructions provided by the supplier. See, Cullen (1987) Meth. Enzymology 43:737; and Gorman et al, (1982) Mol. Cell. Biol. 2:1044. The results are shown in Table 3.

TABLE 3
CAT activity expressed by recombinant PAV viruses

| Sample | ³H cpm |
|---|---|
| Mock-infected | 458 |
| CAT positive control* | 199,962 |
| PAV-CAT2 | 153,444 |
| PAV-CAT6 | 63,386 |

*the positive control sample contained 0.1 Units of purified CAT.

These results show that recombinant PAV viruses, containing an inserted gene, are viable and are capable of expressing the inserted gene.

### Example 7

### Construction of Replication Defective PAV-3 Expressing GFP

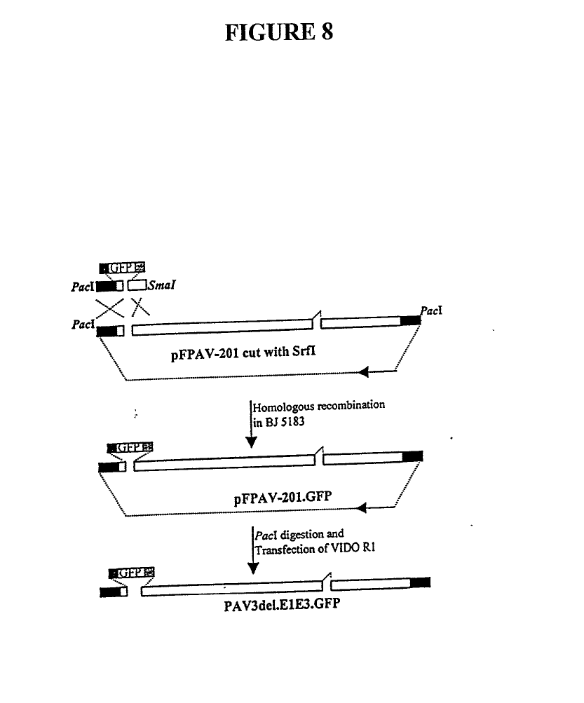

A 2.3 kb fragment containing the CMV immediate early promoter, the green fluorescent protein (GFP) gene and the bovine growth hormone poly(A) signal was isolated by digesting pQBI 25 (Quantum Biotechnology) with BglII and DraIII followed by filling the ends with T4 DNA polymerase. This fragment was inserted into the SrfI site of pPAV-102 in both orientations to generate pPAV-102GFP (FIG. 8). This plasmid, digested with PacI and Smal enzymes, and the fragment containing part of the E1 sequence and the GFP gene was gel purified. This fragment and the SrfI digested pFPAV-201 were used to transform E. coli BJ 5183 to generate the full-length clone containing GFP in the E1 region (pFPAV-201-GFP) by homologous recombination. The recombinant virus, PAV3delE1E3.GFP was generated following transfection of VIDO R1 cells with PacI restricted pFPAV-201-GFP that had the GFP transcription unit in the opposite orientation to the E1. A similar virus with the GFP in the same orientation as E1 could not be rescued from transfected cells. Presence of the GFP gene in the viral genome was confirmed by restriction enzyme analysis. The recombinant virus replicated in VIDO R1 cells two logs less efficiently than the wild type PAV-3.

### Example 8

### Virus Entry and Replication of PAV-3 in Human and Animal Cells

To initially characterize the host species restriction of PAV in vitro, monolayers of 11 cell types from 6 different mammalian species were infected with wild type PAV-3 or PAV3del.E1E3.GFP. ST, VIDO R1 (porcine), 293, A549 (human), MDBK, VIDO R2 (bovine), C3HA (mouse), COS, VERO (monkey), sheep skin fibroblasts or cotton rat lung cells were incubated with 1 pfu/cell of wild type PAV-3 or helper-dependent PAV-3 expressing GFP. The cells infected with wild type PAV were harvested at 2 h and 3 days post-infection, subjected to two cycles of freeze-thaw, and virus titers were determined on VIDO R1 cells. Cells that were infected with the recombinant virus expressing GFP were observed with the aid of a fluorescent microscope for green fluorescence.

A ten-fold increase in virus titers in Vero and COS cells, and a hundred-fold increase in cotton rat lung fibroblasts and VIDO R2 cells, was noticed. No increase in the virus titers was observed with 293, A549, MDBK, sheep skin fibroblasts, dog kidney and C3HA cells. All of these cell types showed bright green fluorescence when infected with PAV3delE1E3.GFP except human cells, which showed a weak fluorescence. In addition, low levels of GFP expression were achieved in human cells with recombinant PAV-3. These observations suggest that virus entry into human cells is limited and/or the human cells are non-permissive for the replication of the virus. These results also demonstrated that GFP was expressed by the PAV-3 vector in cells which are semi-permissive (VERO, COS, Cotton rat lung fibroblasts and VIDO R2), or non-permissive (Sheep skin fibroblasts, MDBK and human cells) for virus replication.

### Example 9

### Insertions in the Regions of the PAV-3 Genome Defined by Nucleotides 145-13,555; 15,284-19,035; 22,677-24,055; 26,573-27,088; and 31,149-34,094

Insertions are made by art-recognized techniques including, but not limited to, restriction digestion, nuclease digestion, ligation, kinase and phosphatase treatment, DNA polymerase treatment, reverse transcriptase treatment, and chemical oligonucleotide synthesis. Heterologous nucleic acid sequences of interest are cloned into plasmid vectors containing portions of the PAV genome (which may or may not contain deletions of PAV sequences) such that the foreign sequences are flanked by sequences having substantial homology to a region of the PAV genome into which insertion is to be directed. Substantial homology refers to homology sufficient to support homologous recombination. These constructs are then introduced into host cells that are co-transfected with PAV-3 DNA or a cloned PAV genome. During infection, homologous recombination between these constructs and PAV genomes will occur to generate recombinant PAV genome-containing plasmids. Recombinant virus are obtained by transfecting the recombinant PAV genome-containing plasmids into a suitable mammalian host cell line. If the insertion occurs in an essential region of the PAV genome, the recombinant PAV virus is propagated in a helper cell line which supplies the viral function that was lost due to the insertion.

### Example 10

### Analysis of Early Region 1 of Porcine Adenovirus

Materials and Methods

Cells and Viruses

VIDO R1 (Reddy et al., 1999(b), J. Gen. Virol. 80:2909-2916) and Swine Testicular (ST) cells (ATCC Cat. No. CRL 1746) were grown and maintained in minimum essential medium (MEM) supplemented with 10% fetal bovine serum (FBS). The PAV strains (wild-type PAV-3 strain 6618) were propagated and titrated in VIDO R1 cells (Reddy et al., 1999(b), supra).

GST Fusion and Antibody Production

The plasmid pE1A was created by amplifying part of E1A (nt 556 to 1222) by PCR and ligating in-frame to glutathione S-transferase (GST) gene in plasmid pGEX-5X-3. To create plasmid pE1Bs, part of E1B^{small} ORF (nt 1470 to 2070) was amplified by PCR and ligated in-frame to the GST gene in plasmid pGEX-5X-3. The plasmid pE1B1 was created by amplifying complete E1B_{large} ORF (nt 1831-3250) by PCR and ligated in-frame to the GST gene in plasmid pGEX-5X-3. The junctions of the sequences encoding GST-E1A, GST-E1B^{small} or GST-E1B^{large} were sequenced to ensure that the coding domains are in frame. The competent Escherichia coli strain BL121 was transformed with pE1A, pE1Bs or pE1B1 plasmids. The fusion protein(s) were induced by addition of 0.1 M isopropyl-β-_{D}-thiogalactoside and purified using sodium dodecyl sulphate (SDS)-polyacrylamide gel electrophoresis (PAGE). Rabbits were immunized subcutaneously with 300 ug of gel purified GST-E1A, GST-E1B^{small} or GST-E1B^{large} fusion proteins in Freund's complete followed by three injections in Freund's incomplete adjuvant at 4-weeks interval.

In vitro Transcription and Translation

The complete coding regions of E1A, E1B^{small} and E1B^{large} were individually cloned into the SmaI site of plasmid pSP64 polyA creating plasmid pSP64-PE1A, pSP64-PE1Bs and pSP64-PE1BI respectively. The plasmid DNAs were transcribed and translated in vitro by using a rabbit reticulocyte lysate coupled transcription translation system in the presence of 50 μCi of [³⁵S]-methionine. The in vitro translated proteins were analyzed with or without immunoprecipitation with the protein specific polyclonal rabbit serum.

Construction of PAV-3 Recombinant Plasmids

The recombinant plasmid vectors were constructed by standard procedures using restriction enzymes and other DNA modifying enzymes.

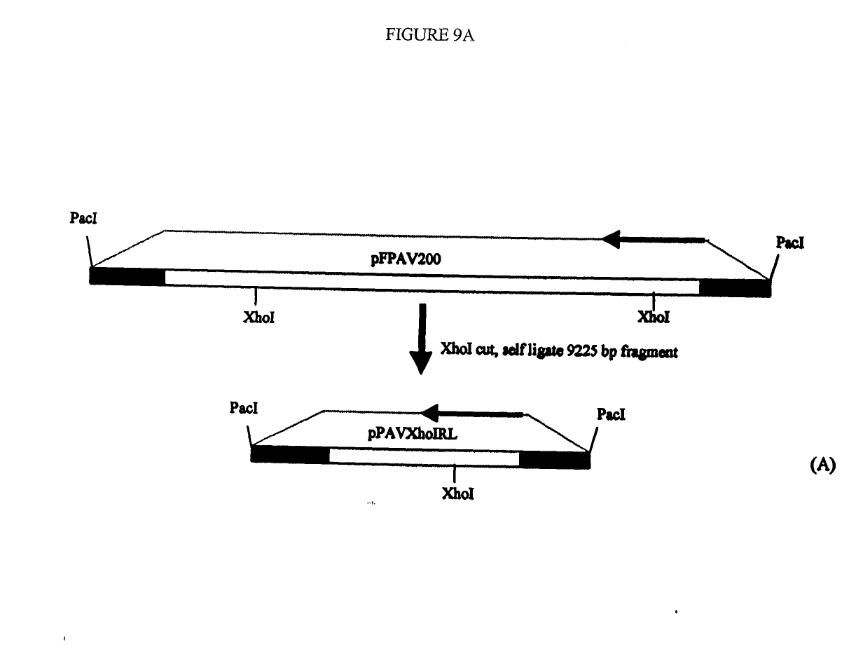

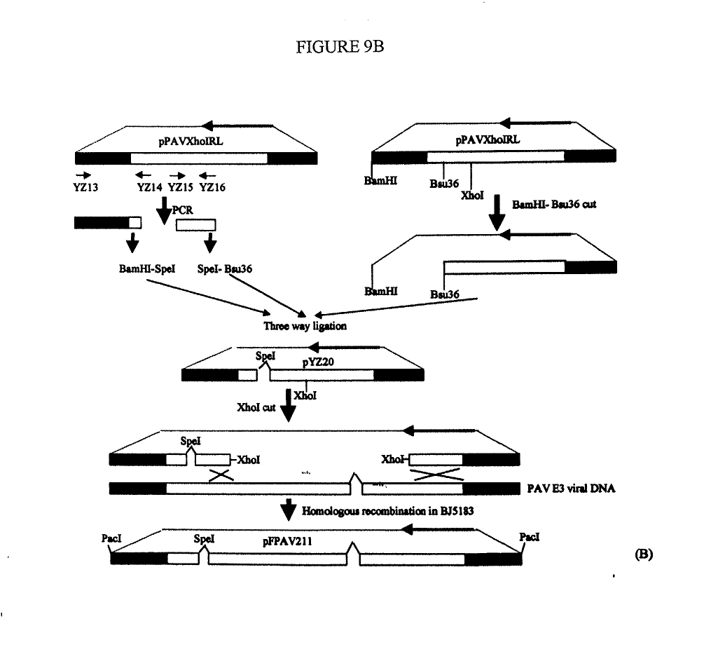

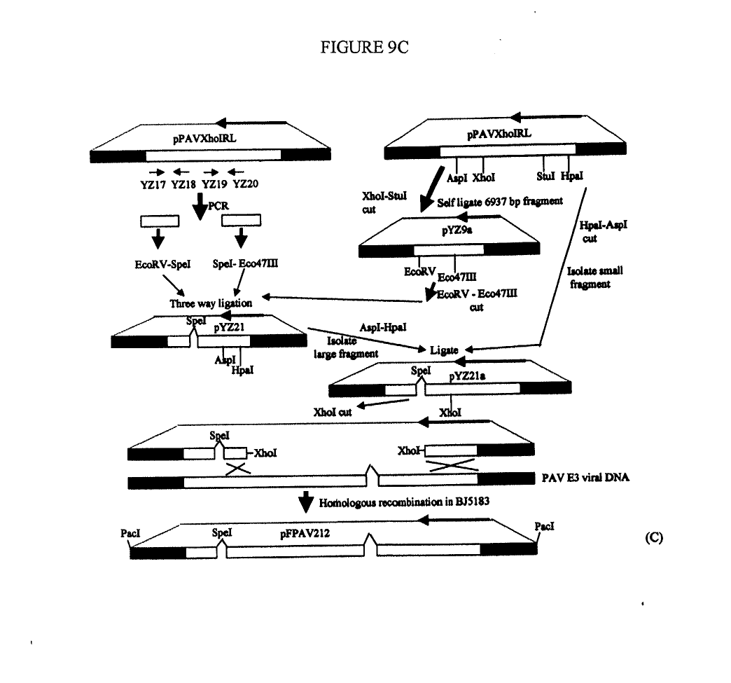

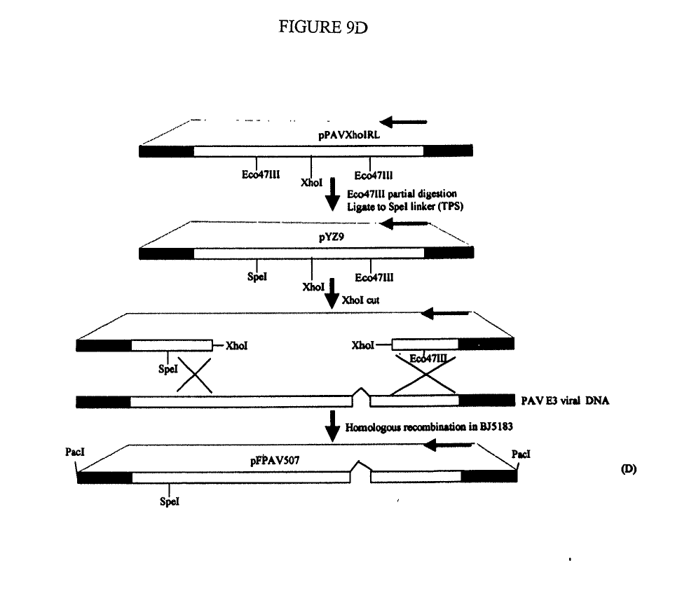

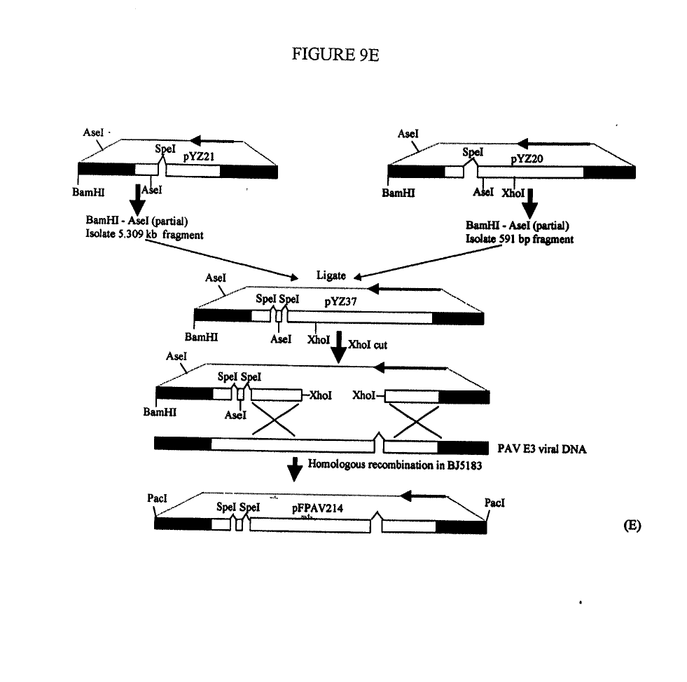

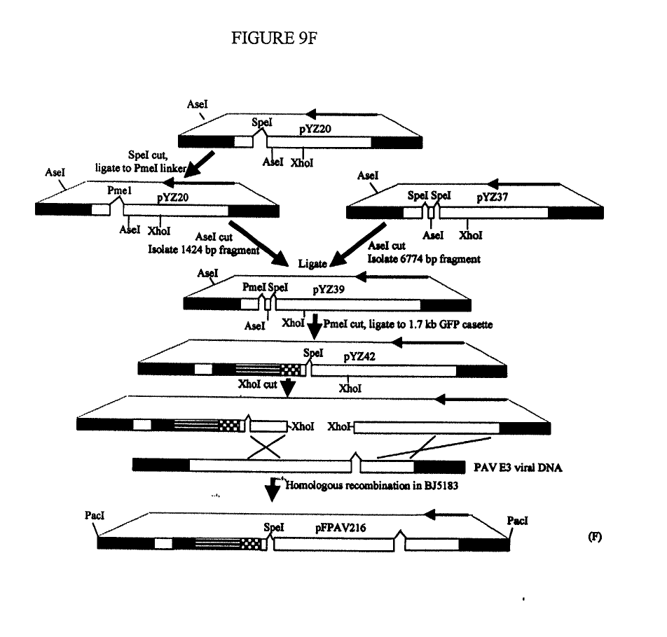

i) Construction of plasmid pFPAV211. A 9.225 kb XhoI fragment (containing vector backbone plus left [nt 004159] and right [nt 31053 to 34094] termini of PAV-3 genome) isolated from plasmid pFPAV200 (Reddy et al., 1999(a), J. Gen. Virol. 80:563-570) was religated creating plasmid pPAVXhoIRL (FIG. 9A). Nucleotide numbers of the PAV-3 genome referred to in this report are according to GenBank accession no. AF083132 (and are the same as in FIGS. 1-1 through 1-10). To delete the E1A region, PAV-3 genome between nucleotides (nt) 0 to 531 was amplified by using primers YZ-13 5′-ATA GGC GTA TCA CGA GGC-3′ and YZ-14 5′-CTG GAC TAG TCT GTT CCG CTG AGA GAA AAC-3′, and plasmid pPAVXhoIRL DNA as a template in a PCR reaction. The PAV-3 genomic DNA between nt 1231 and 1529 was amplified by using primers YZ-15 5′-GTG GAC TAG TCTCAT GCA GCG AACAAC C-3′ and YZ-16 5′-GTA CTA TCA CCT TCC TAA GG-3′, and plasmid pPAVXhoIRL DNA as a template in a PCR reaction. The product of first PCR was digested with BamHI-SpeI and gel purified. The second PCR product was digested with SpeI-Bsu36 and gel purified. The two gel purified fragments were cloned into Bam-HI and Bsu36 digested plasmid pPAVXhoIRL in a three-way ligation. The resulting plasmid pYZ20 carried 700 bp (nt 530 to 1230) deletion in E1A region and an engineered SpeI site. The recombinant PAV-3 genome containing deletions in the E1A and E3 regions (pFPAV21 1) was generated by. homologous DNA recombination in E.coli BJ 5183 between XhoI linearized pYZ20 and genomic DNA of PAV-3 E3 (Reddy et al., 1999(a), supra, FIG. 1B).

ii) Construction of plasmid pFPAV212. A 633 bp fragment (nt 827 to 1460) isolated by PCR amplification (using oligonucleotides YZ-17 5′-ACA GTA ATG AGG AGG ATA TC-3′ and YZ-18 5′-TAG GAC TAG TCC CAC AGA AAA AGA AAA GG-3′ as primers and plasmid pPAVXhoIRL as a template) was digested with EcoRV-SpeI and gel purified. A 403 bp fragment (nt 1820 to 2223 of PAV-3 genome) isolated by PCR amplification (using oligonucleotides YZ-19 5′-ATG GAC TAG TCT TCT GGT GCC GCC ACT A-3′ and YZ-20 5′-CCT AAT CTG CTC AAA GCT G-3′ as primers and plasmid pPAVXhoIRL DNA as a template) was digested with SpeI-Eco471II and gel purified. A 6.947 kb XhoI-StuI fragment of plasmid pPAVXhoIRL was blunt end repaired with T4 polymerase and religated to create plasmid pYZ9a. The two gel purified DNA fragments were ligated to EcoRV-Eco47III digested plasmid pYZ9a in a three way ligation. The resulting plasmid pYZ21 contains 360 bp deletion (nt 1460-1820) in E1B^{small} region and an engineered SpeI site. Finally, a 5.506 kb HpaI-AspI fragment of pYZ21 was ligated to 3.374 kb HpaI-AspI fragment of pPAVXhoIRL to create plasmid pYZ2la. The recombinant PAV-3 genome containing deletions in the E1B^{small} and the E3 region (pFPAV212) was generated by homologous DNA recombination in E. coli BJ5183 between XhoI linearized pYZ21a and the genomic DNA from PAV E3 (Reddy et al., 1999(a), supra; FIG. 1C).

iii) Construction of plasmid pFPAV507. Plasmid pPAVXhoIRL was digested partially with Eco47III and ligated to SpeI linker (triple phase stop [TPS] codon). Plasmid pYZ9 containing SpeI linker inserted in E1B^{large} ORF was selected. The recombinant PAV-3 genome containing deletion in E3 and insertion in E1B^{large} (pFPAV507) was generated by homologous DNA recombination machinery in E. coli BJ5183 between XhoI linearized pYZ9 and the genomic DNA from PAV E3 (Reddy et al., 1999(a); FIG. 1D).

iv) Construction of plasmid pFPAV214. A 0.591 kb BamHI-AseI fragment was excised from plasmid pYZ20 and ligated to 5.309 bp BamHI-AseI (partial) digested pYZb21 to create plasmid pYZ36. Finally, a 4.813 kb HpaI-AspI fragment excised from plasmid pYZ36 was ligated to 3.373 kb HpaI-AspI fragment of plasmid pPAVXhoIRL to create plasmid pYZ37. The recombinant PAV-3 genome containing deletions in E1A, E1B^{small} and E3 region (pFPAV214) was generated by homologous recombination in E. coli BJ5183 between XhoI linearized plasmid pYZ37 and genomic DNA from PAV E3 (Reddy et al., 1999a; Fig. E). The full length plasmid pFPAV214 contained 727 bp (nt 530-1230) deletion in E1A, 360 bp (nt 1460-1820) deletion in E1B^{small} and 597 bp (nt 27405-28112) deletion in E3.

v) Construction of plasmid pFPAV216. Plasmid pYZ20 was digested with SpeI, blunt end repaired with T4 polymerase and ligated to PmeI linker (GTTTAAAC) creating plasmid pYZ39. A 1.424 kb Asel fragment of plasmid pYZ39 was isolated and ligated to 6.774 kb AseI fragment of pYZ37 to create plasmid pYZ40. Finally, a 1.730 kb NruI-PvuII fragment (containing human cytomegalovirus (HCMV) immediate early promoter, GFP gene and bovine growth hormone (BGH) poly(A) signal) was excised from plasmid pYZ41 a (Zhou et al., 2001, Virology) and ligated to PmeI digested pYZ40 to create plasmid pYZ42. The recombinant PAV-3 genome containing GFP expression cassette insertion in E1A region of E1A, E1B^{small} and E3 deleted regions was generated by homologous recombination in E. coli BJ5183 between XhoI linearized pYZ42 and genomic DNA from PAV E3 (Reddy et al., 1999(a); FIG. 1F).

Transfection and Isolation of PAV-3 Mutant Viruses

VIDO R1 cell monolayers seeded in 6-well plate were transfected with 5-10 μg of PacI-digested pFPAV211, pFPAV212, pFPAV214, pFPAV216 or pFPAV507 recombinant plasmid DNAs using the Lipofectin method (Gibco BRL). After 7-10 days of incubation at 37° C., the transfected cells showing 50% cytopathic effects were collected and freeze-thawed three times. Finally, the recombinant virus was plaque purified and expanded in VIDO R1 cells.

Virus Growth Curve

VIDO R1 or ST cells were infected with mutant or wild-type PAV-3 at an MOI of 5. The infected cells, harvested at indicated times post infection were lysed in the infection medium by three rounds of freeze-thaw. Virus titers were determined by serial dilution infections of VIDO R1 cells followed by immunohistochemical detection of DNA binding protein. Titers were expressed as infectious unit (IU), in which 1 IU was defined as one positive stained focus at 3 days post infection.

Western Blot

For Western blot, about 1×10⁶ VIDO R1 or Swine Testicular (ST) cells (ATCC catalogue no. CRL 1746) were infected with recombinant or wild-type PAV-3 at an MOI of 5. At indicated times post infection, the cells were collected and lysed in 100 μl of RIPA (0.15M NaCl, 50 mM Tris-HCl pH 8.0, 1% NP-40, 1% deoxycholate, 0.1% SDS). Proteins were resolved on SDS-PAGE under the reducing condition and electrotransferred to nitrocellulose membrane (Bio-Rad). Nonspecific binding sites were blocked with 1% bovine serum albumin fraction V, and the membrane was probed with the protein specific rabbit polyclonal serum. The membrane was washed and exposed to goat anti-rabbit IgG conjugated to alkaline phosphatase and developed using an alkaline phosphatase color development kit (Bio-Rad).

Radioimmunoprecipitation

VIDO R1 cells in six well plates were infected with wild-type PAV-3 at an MOI of 5. After virus adsorption for 1 h, the cells were incubated in MEM containing 5% FBS. At different times post-infection, the cells were incubated in methionine-cysteine free MEM for 1 h before labeling with [³⁵S] methionine-cysteine (100 μCi/well). After 6 or 24 h of labeling, the cells were harvested. Proteins were immunoprecipitated from cells lysed with modified radioimmunoprecipitation (RIPA) buffer and analyzed by SDS-PAGE as described previously (Tikoo et al., 1993, J. Virol. 67:726-733).

Results

The results of the experimentation disclosed below indicate that E1A is essential for virus replication and is required for the activation of other PAV3 early genes; E1B^{small} is not essential for replication of PAV-3; and E1B^{large} is essential for virus replication. The results also demonstrate expression of a desired transgene in a recombinant porcine adenovirus vector comprising a deletion in E1A, E1B^{small} and E3.

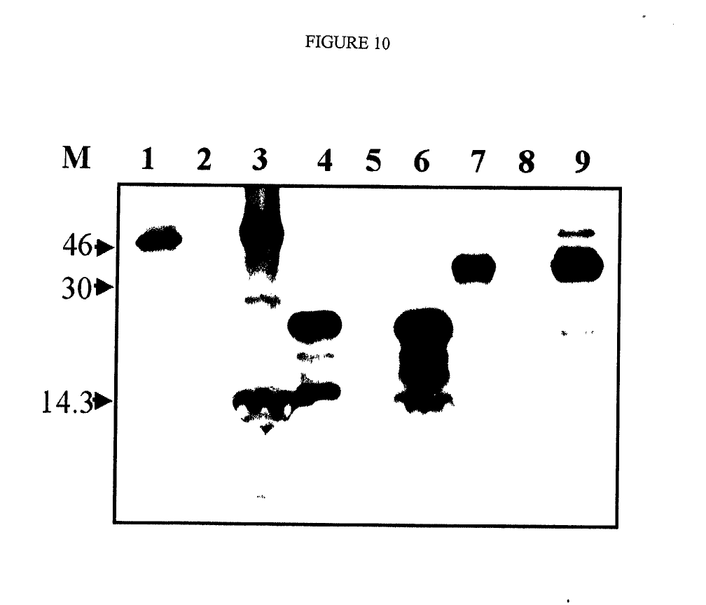

Characterization of PAV-3 μl proteins In order to identify and characterize the proteins encoded by E1 region of PAV-3, anti-E1A, anti-E1B^{small} and E1B^{large} sera were produced by immunizing rabbits with 300 ug of gel purified GST-protein (glutathione S-transferase) fusions. Sera collected after the final boost was analysed by in vitro transcription and translation assays to determine specificity of the antibodies in the rabbit sera. The plasmids pSP64-PE1A, pSP64-PE1Bs and pSP64-PE1B1 were generated in which coding sequence of E1A, E1B^{small} and E1B^{large} respectively, was placed downstream of the SP6 promoter (pSP64polyA vector containing SP6 promoter from Promega, Cat. No. P1241). In vitro translation of pSP64-PE1A RNA resulted in the synthesis of a polypeptide of 35 kDa (FIG. 10, lane 9), which was recognized by anti-E1A serum (FIG. 10, lane 7). In vitro translation of pSP64-PE1Bs RNA resulted in the synthesis of a polypeptide of 23 kDa (FIG. 10, lane 6) which was recognized by anti-E1B^{small} serum (FIG. 10, lane 4). Similarly in vitro translation of pSP64-E1B1 RNA resulted in the synthesis of a polypeptide of 53 kDa (FIG. 10, lane 3), which was recognized by anti-E1B^{large} serum (FIG. 10, lane 1). These proteins were not immunoprecipitated with anti-E1A serum (FIG. 10, lane 8), anti-E1B^{small} serum (FIG. 10, lane 5) or anti-E1B^{large} serum (FIG. 10, lane 2) from reactions in which pSP64polyA (negative control plasmid) was translated in vitro.

To further characterize the proteins and to confirm the specificity of the antisera, radioimmunoprecipitation assays were performed. Anti-E1A serum detected a protein of 35 kDa in PAV-3 infected (FIG. 11A, lane 1-2) but not in mock-infected cells (FIG. 11A, lane 3). The 35 kDa protein was detected at 6 h (FIG. 11A, lane 1) and 24 h (FIG. 11A, lane 2) post infection. Anti-E1B^{small} detected a protein of 23 kDa in PAV-3 infected (FIG. 11B, lane 1-2) but not in mock infected (FIG. 11B, lane 3) cells. The 23 kDa protein was detected at 6 h (FIG. 11B, lane 1) and 24 h (FIG. 11B, lane 2) post infection. Similarly, anti-E1B^{large} serum detected a protein of 53 kDa in PAV-3 infected (FIG. 11C, lane 1-2) but not in mock infected cells. The 53 kDa protein was detected at 6 h (FIG. 11C, lane 1) and 24 h (FIG. 11C, lane 2) post infection.

Generation of PAV-3 E1 Deletion/Insertional Mutants

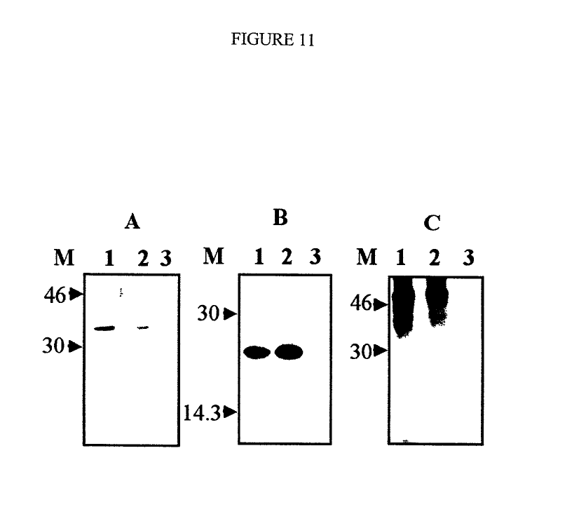

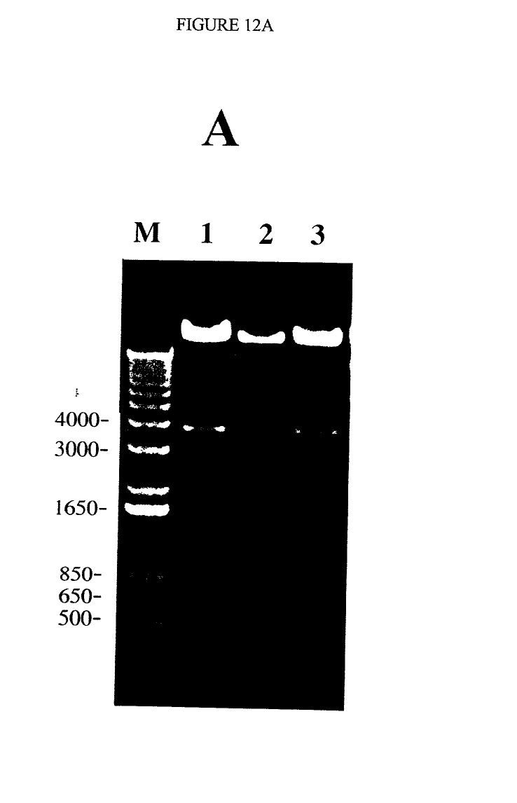

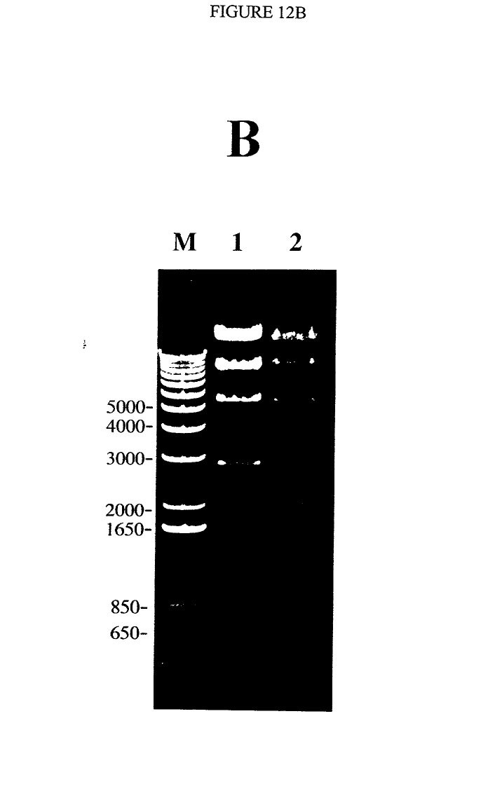

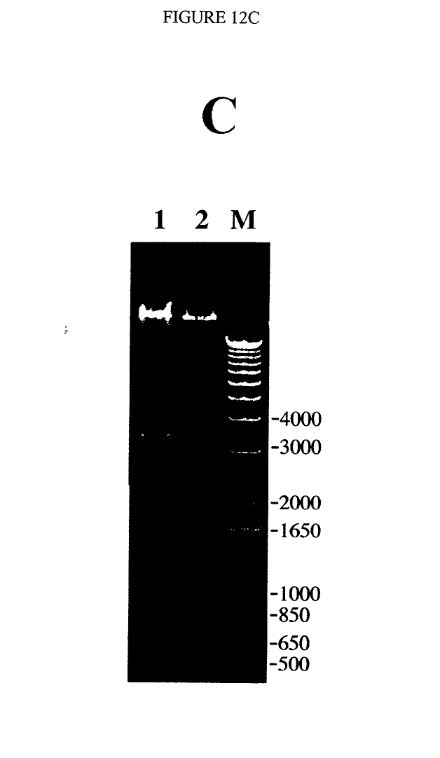

Taking advantage of homologous recombination in E. coli strain BJ5183, three full-length plasmids were constructed a) pFPAV211 containing deletions in E1A (nt 530-1230) and E3 (nt 28112-28709) regions, b) pFPAV212 containing deletions in E1B^{small} (nt 1460-1820) and E3 (nt 28112-28709) regions and c) pFPAV507 containing TPS codon in E1B^{large} (nt 2190) and deletion of E3 (nt 28112-28709) region (all nucleotide numbers are with reference to FIG. 1). The PacI digested pFPAV211 or pFPAV212 plasmid DNAs were transfected into VIDO R1 cells and produced cytopathic effects in 10-14 days. However, repeated transfection of VIDO R1 cells with PacI digested pFPAV507 DNA did not produce any cytopathic effects. The infected cell monolayers were collected and freeze-thawed, and recombinant viruses were plaque purified and propagated in VIDO R1 cells. The recombinant PAVs were named PAV211 (E1A+E3 deletion) and PAV212 (E1B^{small}+E3 deletion). The viral DNA was isolated from virus infected cells by Hirt extraction method (Hirt, 1967, J. Mol. Biol. 26:365-369) and analysed by agarose gel electrophoresis after digestion with restriction enzymes. Since PAV211 and PAV212 genomes contain an additional SpeI site in place of E1A or E1B^{small} regions respectively, the recombinant viral DNAs were digested with SpeI. As seen in FIG. 12A, compared with wild-type PAV-3 (lane 3), the PAV211 (lane 1) or PAV212 (lane 2) genomes contain an additional expected band of 527 bp and 1463 bp respectively.

The ability of PAV211 and PAV212 to produce E1A and E1B^{small} or DNA binding protein (DBP) was tested by Western blot analysis of these proteins from lysates of virus infected Swine Testicular (ST) cells using PAV-3 E1A, E1B or DBP specific anti-serum. DBP anti-serum was prepared in the following manner. A 900-bp fragment coding for the PAV-3 DBP (amino acids 102 to 457) was amplified by PCR using primers PDBP-3 (5′-CGG GAT CCG GCC GCT GCT GCA GCT-3′), PDBP-4 (5′-GCG TCG ACT CAA AAC AGG CTC TCA T-3′) and plasmid PAV3c63 (DBP cDNA) (Reddy et al., 1998, Virology 251:414-426) DNA as a template. The PCR fragment was digested with BamHI-SalI and ligated to BamHI-SalI digested plasmid pGEX-5×-3 (Pharmacia Biotech) creating plasmid pPDBPL8. This plasmid contains the coding region of DBP (amino acids 102 to 457) fused in-frame to the C- terminus of Schistosoma japonicum 26-kD glutathione S-transferase (GST) gene.

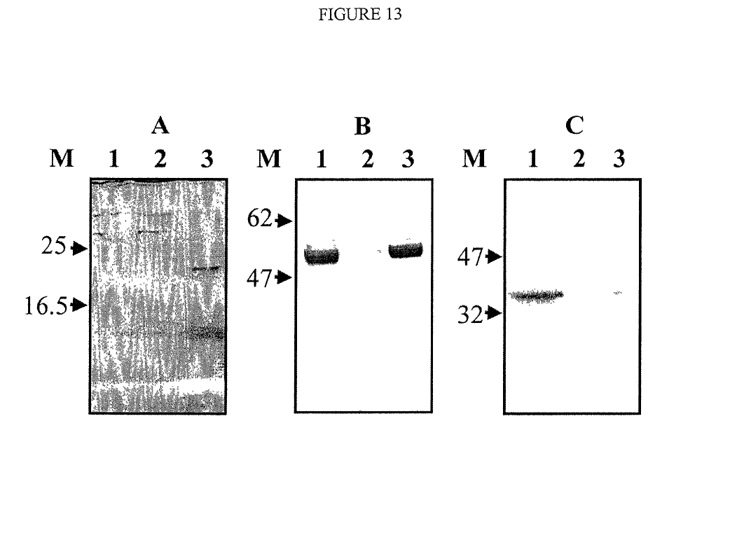

Competent Escherichia coli BL21 were transformed with either plasmid pPDBPL8 or plasmid pGEX-5X-3. Overnight cultures of 100 ml LB broth were inoculated and grown until OD₆₀₀ reached 0.5. Cultures were induced for 4 h in 10 mM IPTG (isopropyl-1-thio-p-D-galactopyranoside). Cells were pelleted and resuspended in 5 ml PBS. The cells were lysed by sonication and the supernatant, collected after centrifugation was applied to GST column. The matrix was washed by the addition of 10 bed volumes of PBS and the fusion protein bound to the column was eluted in glutathione elution buffer. The insoluble protein retained in the cell pellet was purified by sodium dodecyl sulphate (SDS)-polyacrylamide gel electrophoresis (PAGE). The area containing the protein was excised and eluted by incubating the gel slice in 20 ml water at 4° C. overnight. Rabbits were immunized subcutaneously with purified GST-DBP fusion protein in freund's complete adjuvant followed by two injections in Freund's incomplete adjuvant at two weeks interval and DBP anti-serum was collected. Wild-type PAV-3 (FIG. 13C, lane 3) or PAV212 (FIG. 13C, lane 1) infected cells produced an E1A protein of 35 kDa. No such protein was detected in PAV211 (FIG. 13C, lane 2) infected cells. Similarly, wild-type PAV-3 (FIG. 13B, lane 3) and PAV212 (FIG. 13B, lane 1) produced a DBP protein of 50 kDa. No such protein was detected in PAV211 (FIG. 13B, lane 2) infected cells. In addition, wild-type PAV-3 (FIG. 13A, lane 3) infected cells produced an E1B^{small} protein of 23 kDa (FIG. 13B, lane 3). However, no such protein was detected in PAV211 (FIG. 13A, lane 2) or PAV212 (FIG. 13A, lane 1) infected cells.

Construction of E1A+E1B^{small}+E3 Deletion Mutant of PAV-3

In order to increase insertion capacity of the PAV-3 vector, a full length plasmid pFPAV214 carrying deletions in E1A (nt 530-1230), E1B^{small} (nt 1460-1820) and E3 (nt 28112-28709) was constructed by homologous recombination in E.coli BJ5183. Transfection of VIDO R1 cells with PacI digested plasmid pFPAV214 DNA produced cytopathic effects in 7-10 days. The recombinant PAV-3 named PAV214 was plaque purified and expanded in VIDO R1 cells. The viral DNA was extracted and analyzed by agarose gel electrophoresis after digestion with NheI. As seen in FIG. 12B, the wild-type PAV-3 had a fragment of 1.430 kb (lane 2) that was missing in PAV214, which instead had a fragment of 0.737 kb (lane 1).

Construction of E1A+E1B^{small}+E3 Deleted PAV-3 Expressing GFP

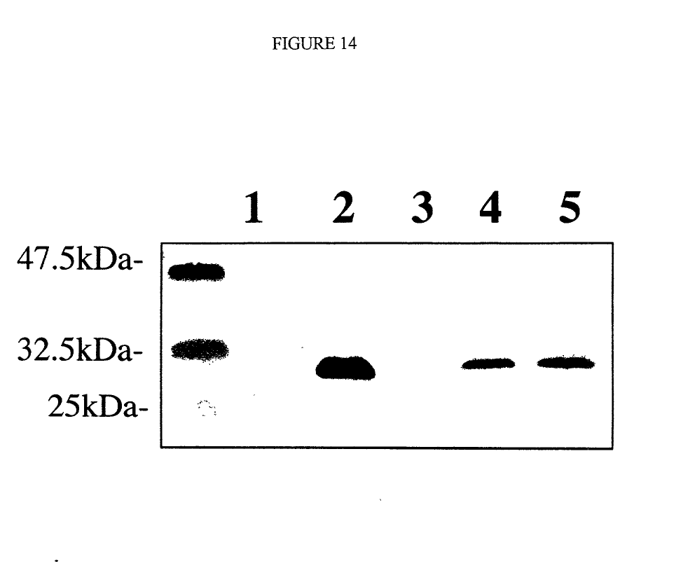

In order to determine if PAV214 genome (E1A, E1B^{small} and E3 deleted) is useful for expression of foreign genes, a recombinant PAV-3 expressing Green fluorescent protein (GFP) was constructed. The full-length GFP gene (flanked by the HCMV promoter and BGH poly (A) signal) was inserted into the E1A region of pFPAV214 in the same transcriptional orientation as E1 (using the homologous recombination machinery of E. coli) creating plasmid pFPAV216. The PacI digested pFPAV216 DNA was transfected into VIDO R1 cells to isolate recombinant virus PAV216. The viral DNA was extracted and analysed by agarose gel electrophoresis after digestion with restriction enzyme. Since there is an AseI site in the CMV promoter, insertion of GFP transcription cassette in the E1A region of PAV214 genome was confirmed by Asel digestion. As seen in FIG. 12C, wild-type PAV-3 had a fragment of 1.274 kb (lane 1) that is missing in PAV216, which instead had two fragments of 0.584 kb and 1.739 kb (lane 2). Expression of GFP protein was confirmed by Western blot using GFP specific polyclonal antibody (Clonetech). As seen in FIG. 14, the GFP could be detected in PAV216 infected VIDO R1 cells at 24 h.p.i. (lane 4) and 48 h.p.i. (lane 5). The size of GFP expressed in cells infected with virus is similar to that of purified GFP protein (lane 2), which is 28 kDa in size. No such protein could be detected in mock-infected cells (lane 1) or wild-type PAV-3 infected cells (lane 3).

Growth Kinetics of PAV2 11, PAV212, PAV214 and PAV216

In order to determine the importance of E1A and E1B^{small} in viral replication, the ability of mutant viruses to grow in VIDO R1 cells and Swine Testicular (ST) cells was compared to that of wild-type PAV-3. Virus infected cells were harvested at different times point infection, freeze -thawed three times and the cell lysates were analyzed for virus titer by DBP detection assay. Virus titers were determined as infectious units (IU) by qualitative DNA binding protein immuno-peroxidase staining. The cell monolayers in 12-well plates were infected with serial dilutions of virus. After adsorption of virus for 90 min, the cells were washed and overlaid with MEM containing 2% FBS and 0.7% agarose (Sigma, low melting temperature). On day 3 post infection, the agarose overlay was carefully removed, the cells were permeabilized with methanol/acetone (1:1 in volume) for 10 min at −20° C. and finally washed with PBS. Non-specific binding sites were blocked by incubating the cells in PBS containing 1% bovine serum albumin for 2 hr at room temperature. The blocking solution was removed and rabbit anti-PAV-3 DBP serum diluted in PBS was added to the plates. After 1 hr incubation at room temperature, the plates were washed with PBS and then processed using Vectastain Elite ABC kit (Vector Laboratories) containing biotinylated anti-rabbit IgG and HRP-steptavidin complex. Finally, the reaction was developed by the addition of substrate 3,3′-diaminobenzidine (DAB) tetrahydrochloride. Titers were expressed as IU in which 1 IU was defined as one positively stained cell/foci at 3 days post infection. Virus titres were also determined using conventional plaque assay.

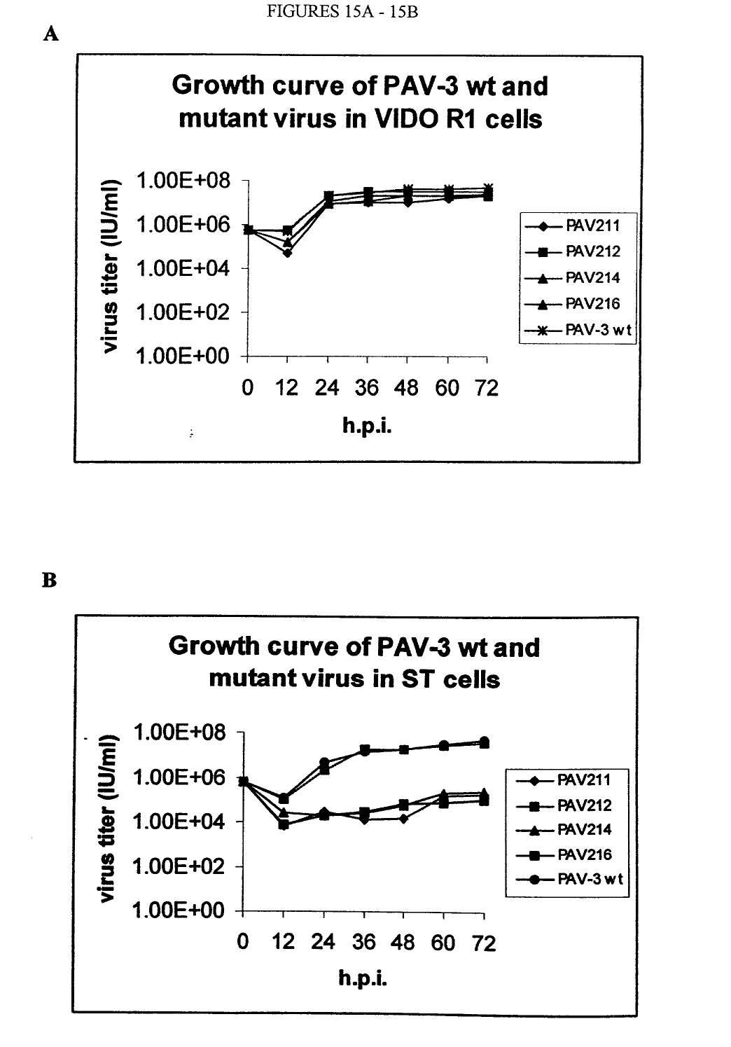

Wild-type PAV-3 titer was 5.2×10⁷ IU\ml at 72 h p.i. on VIDO R1 cells. The titers of mutant viruses were between 2×10⁷-3.2×10⁷ IU/ml, which are quite similar to that of wild-type PAV-3 virus. Therefore, PAV vectors with deletions in E1A and /or E1B^{small} did not have any affect on the ability of PAV-3 to propagate in VIDO R1 cells (E1 complementing cell line) (FIG. 15A). In contrast, we could not observe any progeny virus production in PAV211, PAV214 and PAV216 infected ST cells (E1 non complementing). The virus titers at 72 h.p.i. were never more than 2×10⁵ IU/ml, which was lower than the amount of input virus (FIG. 15B). All of these three viruses carry deletions in E1A region. Most notably, mutant virus PAV212 that carried deletions in E1B^{small} region was able to grow both in complementing and non-complementing cell lines (FIGS. 15A and 15B). At 72 h.p.i. the production of PAV212 in VIDO R1 and ST cells were 3.3×10⁷ IU/ml and 3.9×10⁷ IU/ml respectively.

Deposit of Biological Materials

The following materials were deposited with the ATCC:

Porcine embryonic retinal cells transformed with HAV-5 E1 sequences: VIDO R1 cells were deposited at the ATCC and have ATCC accession number PTA 155.

The nucleotide sequences of the deposited materials are incorporated by reference herein, as well as the sequences of the polypeptides encoded thereby. In the event of any discrepancy between a sequence expressly disclosed herein and a deposited sequence, the deposited sequence is controlling.

While the foregoing invention has been described in some detail by way of illustration and example for purposes of clarity of understanding, it will be apparent to those skilled in the art that various changes and modifications may be practiced without departing from the spirit of the invention. Therefore the foregoing descriptions and examples should not be construed as limiting the scope of the invention.

What is claimed is:

1. A replication-defective recombinant PAV vector, comprising at least one heterologous nucleotide sequence, wherein the PAV vector lacks E1 A function and retains E1B^{small} function.

2. The replication-defective recombinant PAV vector according to claim 1, wherein the vector comprises a deletion of part or all of the E1A gene region.

3. The replication-defective recombinant PAV vector according to claim 1, wherein the vector comprises an insertion in the E1A gene region that inactivates E1A function.

4. The replication-defective recombinant PAV vector according to claim 1 wherein the vector lacks E1 large function.

5. The replication-defective recombinant PAV vector according to claim 4 wherein the vector comprises a deletion of part or all of the E1B^{large} region.

6. The replication-defective recombinant PAV vector according to claim 4 wherein the vector comprises an insertion in the E1B^{large} gene region that inactivates E1B^{large} function.

7. The replication-defective recombinant PAV vector according to claim 1 wherein the vector comprises a deletion of part or all of the E3 region.

8. The replication-defective recombinant PAV vector according to claim 4 wherein the vector comprises a deletion of part or all of the E3 region.

9. A replication-defective recombinant PAV vector, comprising at least one heterologous nucleotide sequence, wherein the PAV vector lacks E1B^{large} function and retains E1B^{small} function.

10. A replication-defective recombinant PAV vector comprising at least one heterologous nucleotide sequence, wherein the PAV vector lacks E1A function and E1B^{small} function and retains E1^{large} function.

11. The replication-defective recombinant PAV vector according to claim 10 wherein the vector comprises a deletion of part or all of the E1A and E1B^{small} regions.

12. The replication-defective recombinant PAV vector according to claim 10, wherein the vector comprises a deletion of part or all of the E3 region.

13. The replication-defective recombinant PAV vector according to claim 10, wherein the vector comprises an insertion in the E1A gene region that inactivates E1A function.

14. The replication-defective recombinant PAV vector according to claim 1, wherein the heterologous nucleotide sequence encodes a therapeutic polypeptide.

15. The replication-defective recombinant PAV vector according to claim 1, wherein the heterologous nucleotide sequence encodes an antigen.

16. The replication-defective recombinant PAV vector according to claim 14, wherein the therapeutic polypeptide is selected from the group consisting of coagulation factors, growth hormones, cytokines, lymphokines, tumor-suppressing polypeptides, cell receptors, ligands for cell receptors, protease inhibitors, antibodies, toxins, immunotoxins, dystrophins, cystic fibrosis transmembrane conductance regulator (CFTR), immunogenic polypeptides and vaccine antigens.

17. A recombinant PAV vector comprising at least one heterologous nucleotide sequence, wherein said vector lacks E1B^{small} function and retains E1A and E1B^{large} function.

18. The recombinant PAV vector of claim 17 comprising a deletion of part or all of the E1B^{small} region.

19. The recombinant PAV vector of claim 17 wherein said vector lacks E3 function.

20. The recombinant PAV vector of claim 19 comprising a deletion in part or all of the E3 region.

21. The recombinant PAV vector of claim 17 wherein said heterologous nucleotide sequence encodes a therapeutic protein.

22. The recombinant PAV vector of claim 17 wherein said heterologous nucleotide sequence encodes an antigen.

23. The replication-defective recombinant PAV of claim 1 wherein said PAV is PAV3.

24. The recombinant PAV of claim 17 wherein said PAV is PAV3.

25. A host cell infected with the replication-defective recombinant PAV according to claim 1.

26. A host cell infected with the recombinant PAV according to claim 17.

27. A method for producing a recombinant PAV that comprises introducing the PAV vector of claim 1 into a helper cell line that expresses E1A function and recovering virus from the infected cells.

28. The method of claim 27 wherein said helper cell line expresses human E1A function.

29. A recombinant mammalian cell line that comprises nucleic acid encoding mammalian adenovirus E1A function and lacks nucleic acid encoding mammalian adenovirus E1B^{small} function.

30. A recombinant mammalian cell line that comprises nucleic acid encoding mammalian adenovirus E1B^{large} function and lacks nucleic acid encoding mammalian adenovirus E1B^{small} function.

31. The recombinant mammalian cell line of claim 29, wherein said nucleic acid encodes human E1A function.

32. The recombinant mammalian cell line of claim 30, wherein said nucleic acid encodes human E1B^{large} function.

33. The recombinant mammalian cell line of claim 29 wherein said cell line is of porcine origin.

34. The recombinant mammalian cell line of claim 30 wherein said cell line is of porcine origin.

35. A method for producing a recombinant PAV, the method comprising:
(a) introducing, into an appropriate helper cell line, a porcine adenovirus vector comprising ITR sequences, PAV packaging sequences, and at least one heterologous nucleotide sequence, wherein said vector lacks E1A function and retains E1B^{small} function;
(b) culturing the cell line under conditions whereby adenovirus virus replication and packaging occurs; and
(c) recovering the adenovirus from the infected cells.

36. The method of claim 35 wherein said PAV is PAV3.

37. The method of claim 35 wherein said heterologous nucleotide sequence encodes an antigen.

38. The method of claim 35 wherein said heterologous nucleotide sequence encodes a therapeutic protein.

39. The method of claim 35 wherein said vector comprises a deletion of part of or all of the E1A gene region.

40. The method of claim 35 wherein said vector comprises an insertion in the E1A gene region that inactivates E1A function.

41. The method of claim 35, wherein said vector lacks E1B^{large} function.

42. The method of claim 41, wherein said vector comprises a deletion of part or all of the E1B^{large} gene region.

43. The method of claim 41, wherein said vector comprises an insertion in the E1B^{large} gene region that inactivates E1B^{large} function.

44. The method of claim 35, wherein said vector comprises a deletion in the E3 region.

45. The method of claim 35, wherein said helper cell line expresses mammalian E1A function and lacks human E1B function.

46. The method of claim 45, wherein said helper cell line expresses human E1A function.

47. The method of claim 46, wherein said helper cell line comprises nucleic acid encoding human E1A function.


SEQUENCE LISTING (18 sequences)

SEQ ID NO: 1 - DNA, 34094 bp, Porcine Adenovirus Type 3
catcatcaataatataccgcacacttttattgccccttttgtggcgtggtgattggcggagagggttgggggcggcgggcggtgattggtggagaggggtgtgacgtagcgtgggaacgtgacgtcgcgtgggaaaatgacgtgtgatgacgtcccgtgggaacgggtcaaagtccaaggggaaggggtggagccctggggcggtcctccgcggggcggggccgagcggcggaaattcccgcacaggtggagagtaccgcgggattttgtgccctctggaccggaccttcgccctccggtgtggcacttccgcaccacacgtccgcggcccggtattccccacctgacgacggtgacaccactcacctgagcggggtgtccttcgcgctgagaggtccgcggcggccgcccgagatgacgtgtgtgggtgtattttttcccctcagtgtatatagtccgcgcagcgcccgagagtcactactcttgagtccgaagggagtagagttttctctcagcggaacagaccctcgacatggcgaacagacttcacctggactgggacggaaaccccgaggtggtgccggtgctggaatgggacccggtggatctgcgcgacccctctccgggggatgagggcttctgtgagccgtgctgggagagtctggtcgatggactgccggacgagtggctggacagtgtggacgaggtggaggtgattgtgactgaggggggtgagtcagaggacagtggtgggagtgccgctggtgactcaggtggctctcagggggtctttgagatggaccccccagaagagggggacagtaatgaggaggatatcagcgcggtggctgcggaggtgctgtctgaactggctgatgtggtgtttgaggacccacttgcgccaccctctccgtttgtgttggactgccccgaggtacctggtgtgaactgccgctcttgtgattaccatcgctttcactccaaggaccccaatctgaagtgcagtctgtgctacatgaggatgcatgcctttgctgtctatggtgagtgtttttggacatttgtgggattatgtggaaaaaaaggaaaaagtgcttgtaagaaatctcatgtgctatttcccattttttgtctttttagaagctgtttctccagcacctcacaggtcgggttccccgggacttggagacctgccaggacgcaagaggaagtactgctatgactcatgcagcgaacaacctttggacctgtctatgaagcgcccccgcgattaatcattaacctcaataaacagcatgtgatgatgactgattgtctgtgtctctgcctatatatacccttgtggtttgcagggaagggatgtggtgactgagctattcctcagcatcatcatcgctctgcttttttctactgcaggctatttcttgctagctcgctgtcccttttctttttctgtgggcatggactatcaacttctggccaagcttactaacgtgaactaccttaggaaggtgatagtacaggggtctcagaactgcccttggtggaaaaagattttttcggacaggtttatcaaggtagtagcagaggccaggaggcagtacgggcaagagttgattgagatttttgtggagggtgagaggggctttggtcctgagttcctgcgggaagggggactgtacgaagaggccgttctgaaagagttggatttcagcaccttgggacgcaccgtagctagtgtggctctggtctgcttcatttttgagaagcttcagaagcacagcgggtggactgacgagggtattttaagtcttctggtgccgccactatgttccctgctggaggcgcgaatgatggcggagcaggtgcggcaggggctgtgcatcatcaggatgccgagcgcggagcgggagatgctgttgcccagtgggtcatccggcagtggcagcggggccgggatgcgggaccaggtggtgcccaagcgcccgcgggagcaggaagaggaggaggaggacgaggatgggatggaagcgagcgggcgcaggctcgaagggccggatctggtttagatcgccgccggcccgggggagcgggtggagaggggagcggggaggaggcgggggggtcttccatggttagctatcagcaggtgctttctgagtatctggagagtcctctggagatgcatgagcgctacagctttgagcagattaggccctatatgcttcagccgggggatgatctgggggagatgatagcccagcacgccaaggtggagttgcagccgggcacggtgtacgagctgaggcgcccgatcaccatccgcagcatgtgttacatcatcgggaacggggccaagatcaagattcgggggaattacacggagtacatcaacatagagccgcgtaaccacatgtgttccattgcgggcatgtggtcggtgactatcacggatgtggtttttgatcgggagctaccggcccggggtggtctgattttagccaacacgcacttcatcctgcacggctgcaacttcctgggctttctgggctcggtaataacggcgaacgccgggggggtggtgcggggatgctactttttcgcctgctacaaggcgctggaccaccgggggcggctgtggctgacggtgaacgagaacacgtttgaaaagtgtgtgtacgcggtggtctctgcggggcgttgcaggatcaagtacaactcctccctgtccaccttctgcttcttgcacatgagctatacgggcaagatagtggggaacagcatcatgagcccttacacgttcagcgacgacccctacgtggacctggtgtgctgccagagcgggatggtgatgcccctgagcacggtgcacatcgctccctcgtctcgcctgccctaccctgagttccgcaagaatgtgctcctccgcagcaccatgtttgtgggcggccgcctgggcagcttcagccccagccgctgctcctacagctacagctccctggtggtggacgagcagtcctaccggggtctgagtgtgacctgctgcttcgatcagacctgtgagatgtacaagctgctgcagtgtacggaggcggacgagatggagacggatacctctcagcagtacgcctgcctgtgcggggacaatcacccctggccgcaggtgcggcagatgaaagtgacagacgcgctgcgggccccccggtccctggtgagctgcaactggggggagttcagcgatgacgatgactgaggatgagtcaccccctcccctcctcttgcaggtacgtggccccgcccagtgggatgggctttggatgggggaggggtgttccctataaaagggggatgggggtggaggcatgcagccccacggggaagcttgtgtggaggatgtcttccgagggtgagatccggacctgcttcatttcagctcgtcttcccagctgggccggcgtgcgtcagggagtggccgggacgaatgtgaacggcggagtggtgggcgcccctgcccagagcggggtgctggcctactcccgcttcgttcagcagcaacagcagcagccggggacggcggcgacggggtctgtgttccgggcggtgtttccatcggtggatctgagcgcggaggtgggcatgatgcggcaggcgctggcggagctgcggcagcagctgcaggagctgcgggaggtggtggagatacagctgcgggccacggcctcggaggcggccgaggaggaagaggaggaggagattgtggtggacgaggaggtggcgcccggcgctggagcgaacaccatggaagaggaggaggatgagatggtcctgacgatgactgtggtgggggaccctgagcctgctggagtggaagcccagccgccaccaccacccaccccggagagcgaccctgcggtgcctgctactaccactaccccgaagcggctcagctacggcgcgagcaagaggagcggtccatgcgcggaggacaactgacgcggactgtggggggaagaagggggaggaggaaagaagaccatggagacgggtgtttgtctttttccagcccaactttattgagaataataataaagcttatggatgtttggaacgataatagcgtgtccagcgttctctgtcttgcagggtcttgtgtatcttctcgaggcaccggtagacctggtgttggacgttgaaatacatgggcatgactccctcggcggggtgcaggtaaagccactggagggctgggtgcggggggcaggtgcagtagatgatccagtcataggcgttctggttgcggtggtggttgaaaatgtccttgaggagcaggctgatggcggtgggcagacccttggtgtaggcattgatgaaccggttgacctgggcgggctgcatgaggggggacatgatgtggtacttggcctggatcttgaggttggagatgttgccgctctggtcgcggcgggggttcatgttgtggaggacgacgaggacggcgtagccggtgcagcgggggaagcgggcgtgcagcttggaggggaaggcgtggaagaacttggcgacccccttgtgtccgccgaggtcctccatgcactcgtcgaggacgatggcgatgggtccgcgggcggcggcgcgggcgaagacgttgcgtgagtcagtgacatcatagttgtgctcctgcatgaggtcctggtagctcatgcggacaaagtctggcatgagggtggcggtctgggggattagggtgtggtccggaccgctgcggtagttgccctcgcagatctgggtctcccaggcgactacctcctgcggggggatcatgtccacctgcggggtgatgaagaaaacagtctccggcgggggggagaggagttgggaggagatgaggttgcggagcagctgggacttgccggagccggtgggaccgtagatgacagcgatgactggctggacctggtagttgagggagcggcaggtgccagccggggtgaggaagggcatgcaggcgttgagggtgtcgcgcaggttgcggttctcttggacgaggtcctgcaggaggtgtcggcctcccagggagaggaggtgggagagggaggcgaaggccttgaggggcttgaggccctcggcgtagggcatgtcctgcagggcctggtggagcacgcgcatgcgctcccagagctcggttacatgtcccacggtatcgtcctccagcaggtctggttgtttctcgggttggggttgctgcgtgagtacggaacgaggcggtgggcgtcgagcgggtggagggtccggtccttccagggccggagggcccgcgtgagggtggtctcggtgacggtgaagggggcggtctggggctgctcggtggccagggtcctcttgaggctgaggcggctggtgctgaaggtggcgcttccgagctgcgcgtcgttcaggtagcactggcggaggaggtcataggagaggtgttgggtggcatggcccttggcgcggagcttgccggggccgcggtgcccgcaagcatcgcaaacggtgtcgcgcagggcgtagagcttgggggcgagcaggaccgtctcggagctgtgggcgtcgctgcggcagcgctcgcactgggtctcgcactcgaccagccaggtgagctgggggttctggggatcgaagacgagggggcccccgttccgcttgaggcggtgtttacctttggtctccatgagctcgcgtccggcgcgggtgaggaagaggctgtcggtgtccccgtagacggagcgcaggggccggtcggcgatgggggtgccgcggtcgtcggcgtagaggatgagggcccactcggagatgaaggcacgcgcccaggcgaggacgaagctggcgacctgcgaggggtagcggtcgttgggcactaatggcgaggcctgctcgagcgtgtggagacagaggtcctcgtcgtccgcgtccaggaagtggattggtcgccagtggtagtccacgtgaccggcttgcgggtcggggggtataaaaggcgcgggccggggtgcgtggccgtcagttgcttcgcaggcctcgtcaccggagtccgcgtctccggcgtctcgcgctgcggctgcatctgtggtcccggagtcttcaggtgggtacgctacgacaaagtccggggtgacctcagcgctgaggttgtctgtttctatgaaggcggaggagcggacggagaggtcgccgcgggcgatggcttcggtggtgcgggcgtccatctggctggcgaagaccaccttcttattgtcgaggcgtgtggcgaaactgccgtagagggcgttggagagaagcttggcgatgctgcggagcgtttggtttctgtcccggtcggccttttccttggcagcgatgttgagctgcacgtagtctcgggcgaggcagcgccactcggggaagatgctgttgcgctcgtccggcaggaggcgcacggcccagccacggttgtggagggtgaccacgtccacggaggtggctacctcgccgcggaggggctcgttggtccagcagaggcggccgcccttgcgggagcagtaggggggcaggacgtccagctggtcctcgtcgggggggtcggcgtcgatggtgaagagggcgggcaggaggtcggggtcgaagtagctgaggggctcggggccgtcgaggcggtcctgccagcggcgggcggccagggcgcggtcgaaggggttgaggggttggccggcggggaaggggtgggtgagggcgctggcatacatgccgcagatgtcatagacgtagaggggctcccgcaggaggccgatgaagttggggtagcagcggccgccgcgcaggctcttcgcggacgtagtcatacagctcgtgggagggcgcgaggaggttcggccgaggtgcggcgcctggggccggctggcgcggtagaggagctgcttgaagatggcgtgggagttggagctgatggtgggcctctggaagacattgaaggcggcgtggggaaggccggcctgcgtgtggacgaaggcgcggtaggactcttgcagcttgcggaccagacgggcggtgacgacgacgtcctgggcgcagtagcgcagggtggcctggacgatgtcgtaagcgtccccctggctctccttcttccacaggtccttgttgaggaggtactcctgatcgctgtcccagtacttggcgtgtgggaagccgtcctgatcgcgtaagtagtcccccgtgcggtagaactcgttcacggcatcgtaggggcagtgtcccttgtccacggccagctcgtaggccgcggcggccttgcggaggctggtgtgcgtgagggcgaaggtgtcccggaccatgaacttgacgtactggtgctgggggtcctcgggggccatgacgccctcctcccagtccgcgtagtcgcggcgcgggcggaaggcggggttgggcaggttgaagctgatgtcattgaagaggatgcggccgttgcgcggcatgaaggtgcgggtgaccaggaaggaggggggcacctcgcggcggtgggcgagcacctgcgcggccaggacgatctcatcgaagcccgagatgttgtggcccacgatgtagacctccaggaagaggggcggcccgcgcaggcggcggcgccgcagctgggcataggccagggggtcctcggggtcgtccggcaggccggggccccgctcctgcgccagctcggcgaggtctgggttgtgggccagcaggtgctgccagagggtgtcggtgaggcgggcctgcagggcgtgccgcagggccttgaaggcgcggccgatggcgcgcttctgcgggcagagcatgtagaaggtgtgggctcgggtctccagcgctgcaggcgggctctggacggccaccacctgcagcgcggcgtccagcagctcctcgtcccccgagaggtggaagaccagcaggaagggcacgagctgctttccgaagcggccgtgccaggtgtaggtctccaggtcataggtgaggaagaggcggcgggtgccctcgggggagccgatggggcggaaggcgatggtctgccaccagtcggccgtctggcgctgaacgtggtggaagtagaagtcccggcggcgcacggagcaggtgtgggcggtctggaagatgcggccgcagtgctcgcacttctgggcctcctggatgctcttgatgaggtggcagcggccctgggtgaagagcaggcggagggggaaggggaggcggggcggcgggccctcgggcggggggtcccagcgcacgtggtgcaggtggtgttgctggcgggtgaccacctggacgaaggtgggcccggcggcgcgggccagctccaccgcggtctggggggtagcctgcaggaggtcggggggcgggcgcaggaggtgcagctggaagaggttggccagggcgctgtcccagtggcggtggtaggtgatgctccagctctccccgtcctgggtggtgccctggaggcggagggtggcgcggcgctcgagcaggagcccccgcgtgccggcctccgcggcctcggcggcggcggccggtctcaggcgggcagctgggccaggggcacgggcgcgttgagctcgggcagcgggaggtggtcgcggcgcagacgcgaggcgtgggcgatgacgcggcggttgatgttctggatctgcgggttcccggagaagaccacgggcccggtgactcggaacctgaaagagagttccacggaatcaatgtcggcatcgtgggtggccacctggcgcaggatctcggacacgtccccgctgttttcgcggtaggcgatgtcctgcatgaactgctcgagctcgtcctcgtccaggtccccgtggccggcgcgctccacggtggcggccaggtcgacggtgatgcggttcatgatggccaccagggcgttctctccgttctcgttccacacgcgactgtagaccagctggccgtcggcgtcccgcgcgcgcatgactacctgggccaggttgagcgccaccaggcggttgaagggcgcctgcaggcgcagggcgtggtgcaggtagttgagggtggtggcgatgtgctcgcagaggaagaagtttatgacccagcggcgcagggtcagctcgttgatgtcgcccaggtcctcgaggcgctgcatgacccggtagaactcgggggcgaagcgaaaaaactcgtgctggcgggccgagaccgtgagctcctcttccagggcggcgatggcctcggccaccgcctgccgcacctcctcctctaaggagggcgggggcgtgctgggtccggccaccgccgcctcttcttcctcttctccctccaggggtggcatctcctcgtcttcttcttctgctgctgctgcctccgcggggacggggggcgcaggccggggacggcgccggcgcaagggcagccggtccacgaagcgctcgatgacctcgccccgcatgcggcgcatggtctcggtgacggcgcggccgccctcccggggccgcagctcgaaggcgcccccgcgcagcgcggtgccgctgcagaggggcaggctgagcgcactgatgatgcagcgtgtcaactctctcgtaggtacctcctgctgttgcagcgcttcggcaaactcgcgcacctgctcttcggacccggcgaagcgttcgacgaaggcgtctagccagcaacagtcgcaaggtaagttgagcgcggtgtgcgtcgggagccggaggtgccggctgacgaggaagtgaaagtaggccgtcttgagctgccggatggcgcgcaggagggtgaggtctttgcggccggcgcgctgcaggcggatgcggtcggccatgccccaggcctcctgctggcagcggccgatgtccttgagctgctcctgcagcagatgtgccacgggcacgtcccggtcggcgtccaggtgggtgcgaccgtagccccgcagggggcgcagcagcgccaggtcggccaccacgcgctcggccaggatggcctgctgcatgcgctgcagggagtctgagaagtcatccaggtccaggaaccggtggtaggcgcccgtgttgatggtgtaggagcagttgcccagcacggaccagttgaccacctggtagtggggctggatgacctcggtgtagcgcagtcgactgtaggcgcgcgtgtcaaagatgtaatcgttgcagaggcgcagcaggtgctggtagcccacgagcaggtggggcggagggtagaggtagaggggccagtgttccgtggccggttggcggggggagaggttcatgagcatgaggcggtggtagcggtagatgaagcgggacatccaggcgatgccgacggcggagacggaggcgcgggtccactggtgggcgcggttccaaatgttgcgcaccgggcggaagagctccacggtgtaaatggattgccccgtgaggcgggcgcagtcgagggcgctctgtcaaaaagaaccgggtgtggttggttggtgtgtggtagcgatctatctttctttgtgatcttggtagtgaagcctgccaggctccagcagggggcgtccgccgtctttccttccttccctatctggaggtgtgtctctgttctcttttttatttcatgtagccatgcatcccgttctgcggcagatgaagccgccggccggcgccctgggcgcggagggggcgacgcgctctcggtcgccctcgccgtcgctgacgcggccgcgcgaggagggggagggcctggcgcggctgtcgggcgcggcggcccccgagcggcacccacgggtgcagctcaagcgagaggccatggaggcctatgtgccgaggcagaatgcgttccgcgagcgaccgggggaggagggggaggagatgagggacctgcggttccgcgcggggcgggagatgcagctggaccgggagcgagtgctccagcccgaggactttgaggggcgcgtggaggaggcggggggagtgagcgcggcgcgggcccacatgagcgcggccagcctggcccaggcctacgagcagacggtacgcgaggaggtcaacttccaaaagaccttcaacaacaacgtgcgcaccctggtgagccgggacgaggtgaccatgggactgatgcacctgtgggactttgtggaggccttcctgcagcacccccggtcccgcgcgctgaccgcgcagctgctgctgatcgcgcagcactgccgggacgagggcatggtgaaggaggcgctgctgagcctgggcgcgcccgagagccgctggctggtggacctggtgaacctgctccagaccattgtggtgcaggagcggtccatgagcctgagcgagaaggtggcggccatcaactactcggtggcgaccctggccaagcactacgcgcgcaagatctccaccttctacatgcgcgcggtggtgaagctgctggtgctggccgacaacctgggcatgtaccgcaacaagcggctggagcgcgtggtcagcacctcgcggcggcgcgagctcaatgacaaggaagctcatgtttggcctccgccgggcgctggccggggagggcgaggaggacctggaggaggaggaggacctggaggaggcggaggaggaggagctggaaagaggaggagttcggtccccggggaccgcggcgcgtgaggtggcagtccccgctgactgcgagcgatgagggtgatgtgtactgatggcaaccatccccctttttaacaacaacagcagcatggcggcgagctctgaagctggggcggcggcggcgggggtgagcgcggcctccctggcgcccgagcgggcgacgcggatgcaggcgctgccctccctggacgagccttgggagcaggctctgcggcgcatcatggcgctgacggccgacgggtctcggcgcttcgcgagccagcccctggccaaccgcatcggggccatcctggaggcggtggtgcctccgcgcacgaacccgacgcacgagaaggtgctgaccgtggtgaacgcgctgctggagacctcggccatccgcccggacgaggccggcatggtgtacgatgcgctgctggagcgggtctcccgctacaacagcggcaacgtgcagaccaacctggaccggctgtcccaggacgtgcggcaggtgatcgcccagcgcgagcgctcgagcgccaacaacctgggcagcctggccgcgctgaatgccttcatcgcctcgctgcccgcaacggtggagcggggccaggagagctacctggggttcctcagcgcgctgcggctgctggtgagcgaggtgccgcagacggaggtgttccgctcggggccgcacaccttcctgcaggcggcgcggaacggttccaagacggtgaacctcaaccaggccatggagaacctgcggcccctgtgggggctgcaggcccccgctggggagcgcgggcacgtgtcctccctgctgacgcccaacacccggctgctgctgctcctggtggctcccttcgcggaggagatgaacgtcagccggagctcctacattgggcacctgctgacactctaccgcgagacgctggccaacttgcatgtggacgagcgcacgtaccaggagatcaccagcgtcagccgggcgttgggcgacgaggacgacgcggcgcggctgcaggccaccctcaacttcttcctgaccaaccggcagcggcggctgccggcggcgtatgccctgaccgccgaggaggagcgcatcctgcgctacgtgcagcaggccgtgagcctgtacctgatgcaggacggggcgacggccacgggcgccctggacgaggccagccgcaacctggagcccagcttctacgcggcgcaccgggacttcatcaaccgcctgatggactacttccatcgcgcggccgcggtggcgcccaactactttatgaatgccgtcctgaacccccgctggctgccctcggagggcttcttcaccggcgtgtatgacttcccggagcaggacgagggggaggagcggccctgggacgcctttgacagcgacgaggagggccgcctcatgctgcggtccgcagcctcctcagagccctcctcctccttcacccccctgcccctgaccgaggagccgccctcgcggccctccaccccggccctctcgcgcgtcccgtcccgggcatcctccctgctctctctggcctctctgggaaagcgggagggaggggactcgctcgcctactcgccggccacgcccacctatggctctcgctggggctcgcgccgctccagcctggccagcggcgccgacagcctggagtgggacgcgctgctggcccctcccaaggatgtgaacgagcacccaggcgccgccgccggccgccgccgccgcgcctcccgctcctccctggaggaggacatcgacgccatcagcagccggctgttcacctggcgcacgcgcgcccaggagatgggcctgcccgtggccagcttctcccgccgccaccagccgcgccccggggccctcgaagacgacgaggaggaggaagactggcgccaggaccggttctttcgcttcgaagcgcccgaggaaaaccccttccgccacatcgcccccaaggggctgtaatgcaaaaaagcaaaataaaaaacccctcccggtccaactcaccacggccatggttgtccttgtgtgcccgtcagatgaggaggatgatgccagcagcgccgccgcagggagcgtcgcctccgccgtcctacgagagtgtggtggggtcttcgctcacggagcctctttatgtgccgccgcggtacctgggccccaccgaggggcggaacagcatccgttattcacagctcccgccgctctacgataccacaaagatctatctgatcgataacaagtcggcggatatcgccagtctgaactaccaaaacaaccacagtgactttctcaccagcgtggtgcagaacagcgacttcacgcccatggaggcgagcacgcagaccatcaacctggatgagcgctcgcgctggggcggggagtttaagagcattctgaccaccaacatccccaacgtgacccagtacatgttcagcaacagcttccgggtgcgcctgatgagcgcgcgcgataaagagacaaatgcccccacctacgagtggttcaccctgaccctgcccgagggcaacttctcggacatcgcggtcatcgacctgatgaacaacgcgatcgtggagaactacctggcggtggggcggcagcagggggtcaaggaggaggacatcggggtgaagatcgacacgcgcaacttccgcctgggctatgacccggagaccaagctggtcatgcccggcagctacaccaacatggcctttcaccccgacgtggtgctggcaccgggctgcgccatcgacttcaccttctcccgcctaaacaacctgctgggcatccgcaagcgctacccctaccaggagggcttcatgctgacctacgaggacctggcggggggcaacatccccgcgctgctggacctcaccacctatgatcaggagaactccagcaccatcaagcccctgaagcaggacagcaagggtcgcagctaccacgtgggcgaggaccccgaggcgggggacaccttcacctactaccgcagctggtacctggcctacaactacggggacccggccacgggcaccgcctcccagacgctgctggtctccccggacgtaacctgcggagtggagcaggtctactggagcctgccggacctgatgcaggacccggtgaccttccggcccagccagacgccgagcaactacccggtggtagccacggagctactgccgctgcgctcccgggccttctacaacacccaggccgtgtactcccagctcctgcagcaggccaccaacaacaccctggtctttaaccgcttcccggagaaccagatcctcctgcgcccgccagagtccaccatcacctccatcagcgagaacgtgccctcgctgacggaccacggcacgctgccgctgcgtaacagcatccccggggtgcagcgggtaaccgtcaccgacgcgcggcgccgcgtgtgtccctatgtgtacaagagtctcggggtggtgaccccgagggtgctcagcagccgaaccttctaaccgacagccctacccgtcacaggggagacagagaaaagacagccagccccgccatggccatcctcgtctcgcccagcaacaactttggctggggactgggcctgcgctccatgtacgggggcgcccgccgcctgtccccggatcaccccgtgatcgtccgacgccactaccgggccaactgggccagtctgaagggacgcgtggcccccagcaccatagcgacaacggatgaccctgtggccgacgtggtcaacgcgatcgccggcgccacccgccgccggcgccgccatcgtcgacgtcggagggccgcgcgcgtctcctccgtggccgtcaccggggacccggtggccgatgtggtcaacgcggtggaggcggtagcccggcgccgccgcgcgcggcgccgttcttcgcgcatgcagaccacgggggaccccgtggcggatgtggtggcggcggtggaagcggtggcgcgccggaggcggagcacccggcggcggcgcaggcgctccgcgccggccatcctgggggtgcgccgcagccgccgcctccgcaaacgcacctcgtcctgagatttttgtgttttgttttttctgcctcccgtgggtgaacaagtccatccatccatccaacatccgtggctgctgtgtctttgtcttttctttgcgttgcgccccagttgagccggcaccgacgcgctcggccatggccatctcgcgccgcgtgaaaaaggagctgctgcaggcgttggcgcccgaggtgtacggggcgcctaagaaggaggagaaggacgtcaaagaggagtccaaagctgaccttaaaccgctgaagaagcggcgcaaggccaagcgggggttgagcgacagcgacgaggtgctggtgctgggcacgcgccccaggcgccgctggacggggcggcgcgtgcgcgcccacctaccgcccggtgccagcctcgcctacgtcccgggtcttcggaggtcgagcgccaccaagcgctctgcggacgagttgtatgcggacacggacatcctgcagcaggcgtcccagcgcctgaacgaatttgcttatggcaagagagcccggcggcagcggcgggcccgcccctcgccgacccccgcgtcccgcggccggaccaccaagcgctcttatgacgaggtcgtggcagacagtgacatcctgcagcaacttggatccggggaccgctccaatgagttctcctatggcaagcggtcgctgctgggggagtcaggagacaccgtcccggctgtggccgtcccgctggaggaaggcaggaaccacacacccagcctgcagccgctcaccgagcccatgcccctggtgtcccctcgcacggccgtcaagcgccgggcgcccgccgacgagcccaccgcctcactggtccccaccgtgcaggtcctggcccccaagcgtcgtctgcaggaggtggtggtggagccgcccgctccagcacccacgccgcccctagccccgcggcggtccagccggcgcatcattctggctccgcgccgggcgggccggccccaggccgtcgtggcgccgcagctcagcgcggccgcggcgctggagcgggcggcggccgccgtgcccctgccaccggacacggaggacgacctggtggagatggcagaggctgtcgccgcgcccgaggtgctgcccagcctccccgtctccatcatgccgcccaccgccacggaggtggccctgcccgtacagaccccactgccgcccgtggcggtggccaagagctccctgacccccggcctccgcgcgctgatgggcaccgagcgggtgccggttccagtcctggaggcgcccctggtggccatgcccgtgctccgggccaccaccgcccgtgccgagcccccgcgccgcgtgccccgcagggccgtgcgggacatcccggccaggcagccccgcacggtatccctgcccgtgctcacggagcccggcccggccaccgcggtcgcctccgtgcgcgcggcagcccaagtcctgcaggcgccccccgcccgaccggccaccgtctccgtgggggtgggcaccgagccggtggtgcagtccatcacggtcaagcggtcaaagcgcctgaccaagcaccatcggggtgcagaccatcgacgtcaccgtgcccaccgtccgcactgtcagcgtgggcaccaacacgccccggctgaggagcgcctcggtgggcgtccagaccgctcccgagacccgctcccagggggtgcaggtggctttccaaccagcgtgctagcccaccgcacacccaggcaggtgcggctgacggcggtggtgccccccaccccgcgcgccccggtggttccggtggcccggcgcccgcggcggttccggtgcctcccccagcccctccagccccgcgcgcgccgcgtgcgcctcgcgcccccagagcgcctcggcgtcgccgccgtaccccggtggcggtggcagcgccgcccgcccgcagcggcggtcccccgccctcggctgccgaggcggcccatcgtgctgcccggggtgcgctatcatcccagtcaggccatggctcccaccgcccaacgcgtcatctggcgttgatttatttttggagacctgactgtgttgtgttccttaaattttttatcctcctcctcctctgctgaagccagacgatgctgacctaccggttgcggctgcccgtgcggatgcggagaccgagactccgcggtgggttccgcgtggcgcctcggcgcagcggcggcaggcggcggtaccgccgggggccgatgaggggtggcatcctgccggcgctggtgcccatcatcgcggcatccatctgggccatccccggcatcgcctcggtggcgatgagtgctagacaacgcaattaacggcgctgctgtgtatgtgtgtcttccatgtgccttccttccttcgttcccaacggaacagcagcaccgtctccatggaggacctaagcttttccgcgttggctccacgctttggcacgcggccggtcatgggcacttggagcgaaatcggcacgagtcagatgaacggcggcgcgctcagctggagcaatatctggagcgggctgaagagctttggtagttctctggcctccacggccaacaaggcctggaacagcgggacggtgacgagcgtgcgcaacaagttgaaggatgccgacgtgcaggggaagataggtgaggtcattgcctccggggtccacggtgccctggacgtggccaaccaggccgtctcccacgccgtggaccgccggtgcaacagcagcagctgcggcagcagcagctcctccgccagcagcagcaacagatgggcctcgtggaaccctcctatgagatggagacagacgagctgcctcctccccccgaggacctcttgcctcctcctcctcctccgccgcctgcctcggccactcccgcgcgccaatcccgcgggacgtcccgccaagcgcccgccgccgcccaggagatcatcatccgctccgacgagccccctccctatgaagagctgtatcccgacaaggccgggatccccgccaccttggagctgcgtcccgagaccaaactgcccgccgtggcccacaataagatgcgccccccgccgccgctcaccaccaccacctcctccgctgccgccgccgcccccgccccggcccccgcggctcctgtgcgtcggcgtccggccgcggctccggccgcggctccggcgagttccaaaggccccccaggtgggggtccgcgcgcgcgggtggcaaaacaaactcaacaccattgtgggactgggtgtccgcacatgcaagcgccgtcgttgttactgagagagacagcatggagaaacaacaatgtctggattcaaataaagacacgcctattcttccacggtgctccgcgctgtgttattttcaacgggctgtttccttttgcatctctgtgccatcgcgccacggggaattccgcaggatggcgacgccgtcgatgatgccgcagtggtcctatatgcacatctccgggcaggacgcgtccgagtacctgtctcccgggctggtgcagttctcccaggcgacggagacctactttaacctgaacaacaagtttaggaaccccaccgtcgcgcccacccacgatgtgacgacggagcgctcgcagcggctgcagctgcgcttcgtccccgtggacaaggaggacactcagtacacatacaagacccgcttccagctggcggtgggcgacaaccgcgtgttggacatggcgagcaccttctttgacatccggggaacgctggaccggggaccctccttcaaaccgtactcgggcaccgcgtacaacatcatggctcccaagagcgctcccaacaactgtcaatatctagaccctaaaggtgaaactgaggctggcaaagttaataccattgctcaagcaagttttgtgggtcctattgatgaaaccacgggagacattaaaattacagaagaagaagacgaagagaccaccatcgatcctttgtatgagccccaaccccagcttggtccaagctcgtggtcagacaatataccttctgcgactagcggagctggaagagttctcaaacagaccacaccgcgtcaaccttgttacggttcttatgcctctccgacaaatattcacggtgggcaaacgaaggatgacaaggttacaccattgtactttacaaacaatcccgccaccgaagccgaagcactcgaagaaaatggattaaagccaaatgtcaccctatactcagaggatgttgacctaaaagcaccagatactcatctggtctatgctgtgaatcaaacccaggaattcgctcaatatggacttggacaacaggccgctccaaacagggccaattacatcggcttcagggacaactttatcgggctgttgtactacaacagcaatggcaaccagggcatgctagccggtcaggcctctcagctcaacgcggtggtcgacctgcaggacaggaatcaccgaactagctaccagctcttcctcgatagcctctatgacaggtcgaggtactttagcctgtggaaccaggccatcgattcttatgacaaggatgtgcgtgtgctggaaaacaatggcgtggaggacgagatgcccaacttttgctttcccatcggcgccatcgagaccaacatgacatttacacagctcaaaaagagtgagaatggtggctcaagagccacaacctggacaaaggagaatggggatgatggcggaaacggagcggagcactacctgggcatcggcaacctcaacgccatggagatcaatctcacggccaacctctggcgcagcttcctctacagcaacgtggcgctgtacctgcctgacaagtacaagttttccccgcccaacgtccccatcgaccccaacacgcactcctatgactacatcaacaagcgcctgcccctcaacaacctcattgatacctttgtcaacatcggggcgcgctggtccccggatgtcatggacaacgtcaaccccttcaaccaccaccgcaactacggcctgcgctaccgctcccagctcctgggcaacggccgctactgcaagttccacatccaggtgccgcaaaagttctttgccctcaagagcctgctgctcctgccgggggcgacctacacctacgagtggtccttccgcaaggacgtcaacatgatcctccagtccacgctgggcaacgacctccgcgcggacggggccaaaatcaacatcgagagcgtcaacctctacgccagcttctttcccatggcccacaacaccgcctccaccctggaggccatgctgcgcaacgacaccaacaaccaaacctttattgacttcctctcctccgccaacatgctctaccccatcccggccaacgtcaccaacctgcccatctccattcccagccgcaactgggccgccttccgcggctggagcttcacgcggctgaagcacaacgagacccccgccctgggctcgcccttcgacccctactttacctactcgggctccatcccctacctggacgggaccttctacctgggccacaccttccgccgcatcagcatccagttcgactcctccgtggcctggccgggcaatgaccgcctgctcactcccaacgagttcgaggtcaagcgcaccgtggacggggagggctacacggtggcccagaccaacatgaccaaagactggttcctggtgcagatgctcgcccactacaacatcggctaccagggataccacctgccagagggctaccgcgaccgcacctactccttcctgcgcaactttgagcccatgtgccgccaggtgcccgactacgccaaccacaaagatgagtacctggaggtgcccaccaccaaccagttcaacagcagcggctttgtatccgcggccttcaccgccggcatgcgcgaggggcacccataccccgccaactggccctacccgctcatcggcgaagacgccgtgcagaccgtgacccagcgcaagttcctctgcgaccgcacgctctggcgcatccccttctcctccaacttcatgtccatgggcaccctcaccgacctgggccagaacctcctctacgccaactcggcccacgccctcgacatgaccttcgaggtcgacgccatggatgaacccaccctcttgtatgttctgttcgaggtctttgacgtctgcggcgtgcaccagccgcaccgaggcgtcatcgaggccgtctacctgcgcacgcccttctccgccgggaacgccaccacctaaggcggagccgcgcaggcatgggcagcaccgaggacgagctccgagccatggcgcgcgacctccagctgccccgcttcctgggcacctttgacaagtccttcccgggcttcttgcaagagtcccagcgctgctgcgccatcgtcaacacggccgcccgccacaccggaggccgccactggctggccgtcgcctgggagcccgcctcgcgcaccttctacttctttgaccccttcggcttctccgaccgggagctcgcccaggtctatgactttgagtaccagcgcctgctgcgcaagagcgccatccagagcaccccggaccgctgcctcacgctcgtcaagagcacccagagcgtgcagggaccgcacagcgccgcctgcggactcttctgcctcctcttcctcgccgcctttgcccgctaccccgacagccccatggcctacaatcccgtcatggacctggtggagggcgtggacaacgagcggctcttcgacgccgacgtccagcccatcttccgcgccaaccaggaggcctgctacgcgttcctcgctcgccactccgcctacttccgcgcccaccgccacgccatcatggaacagacacacctgcacaaagcgctcgatatgcaataaaggctttttattgtaagtcaaaaaggcctcttttatcctccgtcgcctgggggtgtatgtagatggggggactaggtgaacccggacccgccgtcggctcccctccatcccctcttctctcaaaacaggctctcatcgtcgtcctccgttcccacggggaagatggtgttctgcacctggaactggggcccccacttgaactcgggcaccgtcagtggaggccgcgtctgcatcagggcggcccacatctgtttggtcagctgcagggccagcatcacatcgggggcgctgatcttgaaatcacaattcttctgggggttgccgcgcgacccgcggtacaccgggttgtagcactggaacaccagcaccgcggggtgggtcacgctggccagaatcttggggtcttccaccagctgggggttcagcgccgccgacccgctcagcgcgaagggggtgatcttgcaggtctgccggcccagcaggggcacctggcggcagccccagccgcagtcgcacaccagcggcatcagcaggtgcgtctccgcgttgcccatccgggggtagcaggccttctggaaagccttgagctgctcgaaggcctgctgcgccttggagccctccgagtagaagaggccgcaggaccgcgccgagaaggtgttgggggccgaccccacgtcgtggctgcaacacatggccccgtcgttgcgcagctgcaccacgttgcggccccagcggttggtggtgatcttggcgcgctcgggggtctcgcgcagggcgcgctgcccgttctcgctgttgagatccatctccaccagctgctccttgttgatcatgggcagcccgtgcaggcagtgcagcccctccgagccgctgcggtgctgccagatcacgcacccgcaggggttccactcgggcgtcttcagacccgccgccttcaccacaaagtccagcaggaagcgggccatcactgtcagcaggctcttttgcgtgctgaaggtcagctggcagctgatcttgcgctcgttcagccaggcttgggccccgcgccggaagcactccagggtgctgccgtccggcagcagcgtcaggcccttgacatccaccttcagggggaccagcatctgcacagccagatccatggcccgctgccacttctgctcctgagcatccagctgcagcagcggccgggccaccgccgggctcggggtcaccgggcgcggggggcgggccccctcctcttcctccccatcttcgcccttcctcctcgcgggccgcgccgtcgccgctgccgtctcttcagcctcgtcctcctcctcctcgctgaccaggggcttggcacgcgcgcgcttccgccgctcctgcacgggcggagaggccgcgcgcttgcggcctcccccgcgccggctgggggtcgcgacaggagcgtcgtccacaatcagcaccccctcttccccgctgtcatagtcagacacgtccgaatagcggcgactcattttgcttcccctagatggaagaccagcacagcgcagccagtgagctggggtcctccgcggccccgacccttccgccgccaccaccgccgccacctccgcccacgtcaccgccaccttcactgcagcagcggcagcaggagcccaccgaaaccgatgacgcggaggacacctgctcctcgtcctcctcgtcctccgcctccagcgagtgcttcgtctcgccgctggaagacacgagctccgaggactcggcggacacggtgctcccctccgagccccgccgggacgaggaggagcaggaggaggactcgcccgaccgctacatggacgcggacgtgctgcagcgccacctgctgcgccagagtaccatcctgcgccaggtcctgcaggaggccgcccccggcgcagccgcggaggccgccgaggcgccctcggtggcggagctcagccgccgcctggaagcggccctcttctcccccgccacgccgccgcggcgccaggagaacggaacctgcgccccggacccccgcctcaacttctacccggtcttcatgctgcccgaggccctggccacctacctcctcttcttccacaaccaaaagatccccgtcagctgccgcgccaaccgcccacgagccgacgcgcactggcggctgcccagtgggacccccttacctgactatccaaccaccgacgaggtttacaagatctttgagggcctgggggacgaggagccggcctgcgccaaccaggacctgaaagagcgcgacagcgtgttagtcgagctcaagctggacaacccccgcctggcggtggtcaagcagtgcatcgccgtcacccacttcgcctacccggccctggcgctgccacccaaggtcatgagcacgctcatgcagaccctgctggtgcgccgcgcgagcccactccccgacgagggcgagacgcccctcgaggacctcctggtggtcagcgacgagcagctggcccgctggatgcacacctcggaccccaaggtcctggaggagcggcgcaagaccgtcaccgccgcctgcatggtcacggtgcagctccactgcatgcacaccttcctcacctcccgcgagatggtgcgccgcctcggagagtgcctccactacatgttccgccagggctacgtcaagctagctagcaagatcgccaatatggaactctctaacctggtctcctacttgggcatgctgcacgaaaacaggctcggtcagcacgtgctccaccacaccctcaagcatgaggcgagacgcgactacgtccgggacaccatttacctatacctggtctatacctggcagaccgccatgggggtctggcagcagtgcctcgaggaccgaaacctgcgcgccctggaaacgtctctggctcgcgctcgccagagcctgtggacgggctttgatgagcgcactatcgcgcaggacctcgccgcgttccttttccccaccaagctcgtagagaccctgcagcgctcgctccccgactttgccagccagagcatgatgcatgccttccgctccttcgtcctcgagcgctccggcatcctgcccgccgtctgcaacgcgctcccctctgactttgtgcccaccgtctaccgcgagtgcccgccgcccctctgggctcactgctacctcctgcgcctcgccaacttcctcatgtaccactgcgacctcgccgaggacacctccggcgagggcctctttgagtgctactgccgctgcaacctctgcgcaccgcaccgctgcctcgccaccaacaccgccctcctcaacgaggtgcaagccatcaacacctttgagctccagcggccccccaagcccgacggcaccctgccaccgcccttcaagctgacccccggtctctggacctccgccttcctccgccactttgtctccgaggactaccactcggaccgcatcctcttctacgaggacgtgtcccgcccccccagggtggagccctccgcctgcgtcatcacgcactcggccattctcgcgcaattgcatgacatcaaaaaggccagggaagagtttttgctgaccaaaggccacggcgtctacctagacccccacaccggagaggagctcaacaccgccgccccgtccaccgcccaccatgccgcccctccggaggaagcccatccgcagcagcaccagcaccagcagcagccgagccaccgccgccgccaccaccgctccagctacgcagaccgtgtccgaagcgagctccacgcctacggcggtgcgaccggttcctcccgcgaccctgtctctggcggatgctctgccagaggaacccactcccgcgatgctgctcgaagaagaggctctcagcagcgagaccagcggcagctccgaaggcagtttgctcagtaccctcgaggaactggaggaggaggaggaaccggtcacaccgacgaggccatccaagccctcctacaccaacagcagcagcagcaagagcatcagccagcgcaggaactccgtcgtccccagcgaggctcgtagatggaatcagacatccatccaccggagtagccagccaggtaggacacctccgccctcggcccgccgacgctcctggcgccgctaccgccacgacatcctctcggccctggagtactgcgccggagacggagcctgcgtgcgccggtacctactctaccaccacaacatcaacatcccttccaagatcatccgttactacaaatcctcttcccgttccagcgatctccaggaaggccgcagcagcggcggcagcagaaccagcccacgtcagccagctgagagctaagatcttccccacgctgtacgccatcttccagcagagccgcggcggccaggacgccctcaaaatcaggaaccgcaccctgcgctccctcaccaagagctgtctgtatcaccgcgaggaggccaagctggaacgcacgctctcggacgcagaagctctcttcgagaagtactgcgctcggcagcggcagacccgccggtatttaaggagcggaccctgcgtgcggacacaccatgagcaaacaaatccccaccccgtacatgtggtcttatcagccacaatctgggcgtgccgccggtgcctccgtcgattactccacccgcatgaattggctcagtgccgggccttccatgattggccaggtcaatgacatccgacacaccaggaaccagattctcattcgccaggcccttatcaccgagacgccacgccccgtccaaaatcccccgtcctggcccgccagcctgttgcctcagatgacgcaaccgcccacccacctgcacctgccgcgtaacgaaattttggaaggcagactgactgacgccggcatgcaattagccgggggcggagccctcgcacccagagacttatatgccctgaccctccgcggcagaggcatccagctcaacgaggacctacccctctcggcgagcactctccggccggacggcatcttccagctcggaggcggaggccgctcctccttcaaccccaccgacgcctacctgacgctgcagaactccagctcccttccccgcagcggcggcatcggcagcgagcaatttgtccgcgagttcgtgcccacggtctacatcaaccccttctccggaccgcccgggacctaccccgaccagttcatcgccaactacaacatcctaacggactctgtagcaggctatgactgacggtccccagggtcagcagcggctgcgggagctcctcgaccagcaccgccgccagtgccctaaccgctgctgcttcgccagggaagggattcacccggagtacttttgcatcacccgcgagcactttgaggccgagtgcatccccgactctctgcaagaaggccacggtctgcgcttcagcctccccacgcgctacagcgaccgccgccaccgcgatggagaccgcaccatcctcacttcgtactactgcggccctgcttctttcaaagttcgctgtctctgcggccatcctgctcctcaccctcttcttctcgaccttctgtgtgagctgtacaaccgctcgtagcgtcagcccctacacctcccctcgcgtccaatttctgtccgacatagaaccagactctgactcttactcgggctctggctctggggacgatgaagattatgaatatgagctggctaccaacacaccgaacgaagacattctaggcagcatagtcatcaacaaccagatcgggcccaagaccctggccctgggatacttttatgccgccatgcagtttgtcttctttgccatcatcatcatcgtcctcatcctctactaccgccgctacgtgctggccaccgccctcatcgtgcagcgccagatgtggtcctccgaggccgtcctgcggaaaaccttctcggccaccgttgtggttactcccccaaaacaagtcaccccctgcaactgctcctgccgcttcgaggagatggtgttctactacaccacctccgtcttcatgccctggtgggcctcatcctcctgctcaccgccatggtccgcctggccaactggatagtggatcagatgcccagcaggaaccgcgccccgccgctgccaccgcccctcacctatgtgggaccctgcgccgaggaccacatctacgatgagccaaccgtagggcaatacgtacagatgaagtagctccccctctttcccattcccccatttttctctattcaataaagttgcttacctgagttcatccacactcggtctgccagtgcagtctatccatgcgccgttttccatactcacatagcgcagccgcgcacgcctcgccaggtgacgaaactgtcgaaatgtaacatttcgcgcttctgtcagcagcaccccgttatagaccagttccaccatgggaccgaagaagcagaagcgcgagctacccgaggacttcgatccagtctacccctatgacgtcccgcagctgcagatcaatccacccttcgtcagcggggacggattcaaccaatccgtggacggggtgctgtccctgcacatcgcaccgcccctcgtttttgacaacaccagggccctcaccctggccttcgggggaggtctacagctctcgggcaagcagctcgtcgttgccaccgagggctcggggctaaccaccaacccggatggcaagctggttctcaaagtcaagtcccccatcaccctgaccgccgagggcatctccctgtccctgggtcccggtctttctaactcagagaccggcctcagtctgcaagtcacagctcccctgcagttccagggcaacgccctcactcttcccctcgccgccggtctccaaaacaccgatggtggaatgggtgtcaaactggggagcggtctcaccacggacaacagtcaggcggtgaccgttcaggtgggaaatggacttcagctgaacggcgaaggacaactcaccgtccccgccacggcccctttagtctcagggagcgcaggcatctctttcaactactccagcaatgacttcgtcttagacaatgacagtctcagtttgaggccaaaggccatctctgtcacccctccgctgcagtccacagaggacacaatctccctgaattattctaacgacttttctgtggacaatggcgccctcaccttggctccaactttcaaaccctacacgctgtggactggcgcctcacccacagcaaatgtcattctaacaaacaccaccactcccaacggcacctttttcctatgcctgacacgtgtgggtgggttagttttgggttcctttgccctgaaatcatccatcgaccttactagtatgaccaaaaaggtcaattttatttttgatggggcaggtcggcttcagtcagactccacttataaagggagatttggatttagatccaacgacagcgtaattgaacccacagccgcaggactcagtccagcctggttaatgccaagcacctttatttatccacgcaacacctccggttcttccctaacatcatttgtatacattaatcagacatatgtgcatgtggacatcaaggtaaacacactctctacaaacggatatagcctagaatttaactttcaaaacatgagcttctccgcccccttctccacctcctacgggaccttctgctacgtgccccgaaggacaactcaccgtccccgccacggcccctttagtctcagggagcgcaggcatctctttcaactactccagcaatgacttcgtcttagacaatgacagtctcagtttgaggccaaaggccatctctgtcacccctccgctgcagtccacagaggacacaatctccctgaattattctaacgacttttctgtggacaatggcgccctcaccttggctccaactttcaaaccctacacgctgtggactggcgcctcacccacagcaaatgtcattctaacaaacaccaccactcccaacggcacctttttcctatgcctgacacgtgtgggtgggttagttttgggttcctttgccctgaaatcatccatcgaccttactagtatgaccaaaaaggtcaattttatttttgatggggcaggtcggcttcagtcagactccacttataaagggagatttggatttagatccaacgacagcgtaattgaacccacagccgcaggactcagtccagcctggttaatgccaagcacctttatttatccacgcaacacctccggttcttccctaacatcatttgtatacattaatcagacatatgtgcatgtggacatcaaggtaaacacactctctacaaacggatatagcctagaatttaactttcaaaacatgagcttctccgcccccttctccacctcctacgggaccttctgctacgtgccccagagtgcctagagaaccctggccgtcagccggcctcccccttcccaggccacccggtacaccacccgctccatgtttctgtatgtgttctcctcccgccgcttgtgcagcaccacctcccgctgctcgagctgaggatccgtgatggacacaaagccaggaagacacatcctcagctccgtgggggcgtccaacaactgtttatgtaaaggaaaataaagactcagagaaaatccaagttcatatgatttttcttttattgattgggggaattgattcaggtggggtgtgcataatcacaaaaatcacatcagcaggtacacacctgagacatcagacaggggtaaggacagcgcctcagcttctggaacagacatcagaaatatttaatctgctggtagctaacactccttcccaacaccatacactcctggagggccctctgcctctcctcctcccgctccgcgtccctctgccgggaccaccactccccctccgtgaactgctgcttcctcccccgccgctgcgccccgatggcctccgccgccagcttcagccagtgccgcaagcgctgggcgcagcgccgagccaccggctcgctcagctcgtggcagcgccggcacaccagcactatgtaattggcatagtccccgtcacagtagatgacctccccccagtggaacatgcgcaacagcttcagatcacagtcatacatgatctttatgtacatcaggtgggcgcctcgaaacatcacactgcccacgtacatcacgcgactcacgctgggcaggttcaccgcctccctgaaccaccagaagatgcgattgtactcgcagccccggatgatctcgcgcatcagggagcgcatcaccacctgccccgcgcggcactccagactggaccttttcagacagtggcaatgaaagttccacagcgtcgcgcccgcacagcgtctccgggctgaaacatatctgctccagctccaaccccccacacaggctgtactgcaggaaaatccattcttgatgggaaaggatgtagcgccaggggaccacaatctccaaacagggaacaaaacataccgcggcccggctgttgcgcacggcccccaccggatgcaacgtgctcacggagcagatacgggtgggacagcggcccacgtctcatagcaagtcaagtccggaagtggcacggggttcgccaccactgctactgctgccgctgcgccaccagctccatcggctcctccatcctcctcctgttccatcggctgaggtgctgcctcctcctcctcctgccgctgctccatcatgctcgtctgcggtcatcaggagtcaaaaaattcattggccaccgcacgcagagagaacatggagcgcaggggcccaggtgcccggcccgtgcgctcgctcaactcccccagcaggtactcatagagatgctcctccaaatccaccgcaaaccaggcatgcagaaactcttccgttcgaggaccgcccacggtaaagacatagccctcccgcaccttcaccgctgccagctgcacgcgctcatgtcgctgggagtacacccggacccgggcctggatgtactccagcacctgatcgctcagacacctcacagagatgccagcctgagccagcttctcatagagaggtggctgaatcttgagcttgaagcagcgagcggctaggcactccccgcccccttggaacagggcggccgggtcagccatggacttcctctacatccggggtcctggccacctcacaaactatctggccaatcgcctgaccacgggtcaccaggtaaggatgatgtccgttgttgcgaatgagaatgctcagaggtgactcggtagcgttatcaatcacgtccccaaaggtccaaaggtcccagttagaagtcaggtgcttcagaccgcagacacgcccatagcaaccagtgggaaaagccagcaagagatccgtgggcacatgcaccgaagctcccgcaggaatctccacccactccgaggcgtagaccgtgtaagctacacaccccgcctcccgagtgggagcagaagcattctcgctcagccgaaagaacttcagggtggcctgcatatcctcttttactcacttgttagcagctccacacagaccagggttgtgttggcgggaataggcagcaggggtacgtccccagtgagggacacctggatggggggcagaggattgatgccaggaagcagcaggtactgggaaacagagaccagatccctcctctgaaaaatctcgctcagtcggacaaacacagcaaacccagtgggcacgtagactagcacattaaaaaggatcacgctgggctgttctgacgtcagcaccagatgtcgggacgtgcgcagatgaatgcggttctgatgaattaccggaggcctctcacccgcagccaacagcagaccgggctgctgatgcggtcccgcagacatatatgagttcaatgtgtgtcttttttctaaacgtctagtgagtgtgctcgtcctgctcctgccaatcaaaatccgggcaccagggctggtggttggacccgatgaagaagcgaggagaggcggcctcctgagtgtgaagagtgtcccgatcctgccacgcgaggtaggcgaagtacagatagagcacggcgagaacagtcagcaccgcggccagcagcagtcggtcgtgggccatgagagggggctgatgggaagatggccggtgactcctctcgccccgctttcggtttctcctcgtctcgctctcagtgtctctctctgtgtcagcgccgagacgagtgtgagcgaacaccgcgagcgggccggtgatatacccacagcggatgtggccacgcctgcggtcggttaatcagtaccccatcgtccgatcggaattcccccgcctccgcgttaacgattaacccgcccagaagtcccgggaattcccgccagccggctccgccgcgacctgcgactttgaccccgcccctcggactttgaccgttcccacgccacgtcattttcccacgcgacgtcacgttcccacgctacgtcacacccctctccaccaatcaccgcccgccgcccccaaccctctccgccaatcaccacgccacaaaaggggcaataaaagtgtgcggtatattattgatgatg

SEQ ID NO: 2 - DNA, 44 bp, Porcine Adenovirus Type 3
gcggatccttaattaacatcatcaataatataccgcacactttt

SEQ ID NO: 3 - DNA, 32 bp, Porcine Adenovirus Type 3
cacctgcagatacacccacacacgtcatctcg

SEQ ID NO: 4 - DNA, 32 bp, Porcine Adenovirus Type 3
cacctgcagcctcctgagtgtgaagagtgtcc

SEQ ID NO: 5 - DNA, 20 bp, Porcine Adenovirus Type 3
gactgacgccggcatgcaat

SEQ ID NO: 6 - DNA, 27 bp, Porcine Adenovirus Type 3
cggatcctgacgctacgagcggttgta

SEQ ID NO: 7 - DNA, 27 bp, Porcine Adenovirus Type 3
cggatccatacgtacagatgaagtagc

SEQ ID NO: 8 - DNA, 20 bp, Porcine Adenovirus Type 3
tctgactgaagccgacctgc

SEQ ID NO: 9 - DNA, 18 bp, Porcine Adenovirus Type 3
ataggcgtatcacgaggc

SEQ ID NO: 10 - DNA, 30 bp, Porcine Adenovirus Type 3
ctggactagtctgttccgctgagagaaaac

SEQ ID NO: 11 - DNA, 28 bp, Porcine Adenovirus Type 3
gtggactagtctcatgcagcgaacaacc

SEQ ID NO: 12 - DNA, 20 bp, Porcine Adenovirus Type 3
gtactatcaccttcctaagg

SEQ ID NO: 13 - DNA, 20 bp, Porcine Adenovirus Type 3
acagtaatgaggaggatatc

SEQ ID NO: 14 - DNA, 29 bp, Porcine Adenovirus Type 3
taggactagtcccacagaaaaagaaaagg

SEQ ID NO: 15 - DNA, 28 bp, Porcine Adenovirus Type 3
atggactagtcttctggtgccgccacta

SEQ ID NO: 16 - DNA, 19 bp, Porcine Adenovirus Type 3
cctaatctgctcaaagctg

SEQ ID NO: 17 - DNA, 24 bp, Porcine Adenovirus Type 3
cgggatccggccgctgctgcagct

SEQ ID NO: 18 - DNA, 25 bp, Porcine Adenovirus Type 3
gcgtcgactcaaaacaggctctcat